(19) United States
(12) Patent Application Publication
(10) Pub. No.: US 2021/0380556 A1
(43) Pub. Date: Dec. 9, 2021

(54) HETEROARYLAZOLE COMPOUND AND PEST CONTROL AGENT

(71) Applicant: Nippon Soda Co., Ltd., Chiyoda-ku, Tokyo (JP)

(72) Inventors: Keita SAKANISHI, Kanagawa (JP); Norifumi SAKIYAMA, Kanagawa (JP); Hikaru AOYAMA, Kanagawa (JP); Takao IWASA, Kanagawa (JP); Maki MATSUI, Kanagawa (JP); Tomomi KOBAYASHI, Kanagawa (JP); Daisuke USHIJIMA, Kanagawa (JP)

(73) Assignee: Nippon Soda Co., Ltd., Chiyoda-ku, Tokyo (JP)

(21) Appl. No.: 17/280,655

(22) PCT Filed: Sep. 30, 2019
(86) PCT No.: PCT/JP2019/038480

§ 371 Date: Mar. 26, 2021

(30) Foreign Application Priority Data
Oct. 2, 2018 (JP) 2018-187675
Oct. 29, 2018 (JP) 2018-202997

(51) Int. Cl. C07D 401/14; A01N 43/54; A01N 43/653; A01N 43/50; C07D 401/04; A01N 25/00; A01N 25/12

(52) U.S. Cl. CPC C07D 401/14; A01N 43/54; A01N 43/653; A01N 25/12; C07D 401/04; A01N 25/002; A01N 43/50

(57) ABSTRACT

An object of the present invention is to provide a heteroaryl azole compound that is excellent in pest control activity, particularly, insecticidal activity and/or miticidal activity, is excellent in safety, and may be industrially advantageously synthesized. The compound of the present invention is a compound represented by the formula (I), an N-oxide compound, stereoisomer, tautomer or hydrate thereof or a salt of any of these compounds. In the formula (I), A represents CH or a nitrogen atom; B¹ represents CX¹ or a nitrogen atom; X¹, X² and X³ each independently represent a hydrogen atom, a substituted or unsubstituted C1-6 alkyl group, a substituted or unsubstituted C2-6 alkenyl group, or the like; R¹ represents a substituted or unsubstituted C1-6 alkylthio group, a substituted or unsubstituted C1-6 alkylsulfinyl group, or the like; R² represents a substituted or unsubstituted C1-6 alkyl group; and R represents a substituted or unsubstituted C1-6 alkyl group.

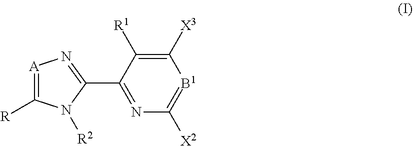

### TECHNICAL FIELD

The present invention relates to a heteroaryl azole compound and a pest control agent. More specifically, the present invention relates to a heteroaryl azole compound that has excellent insecticidal activity and/or miticidal activity, is excellent in safety, and may be industrially advantageously synthesized, and a pest control agent containing the same as an active ingredient. The present application claims the priority of Japanese Patent Application No. 2018-187675 filed on Oct. 2, 2018 and Japanese Patent Application No. 2018-202997 filed on Oct. 29, 2018, the contents of which are incorporated herein.

### BACKGROUND ART

Various compounds having insecticidal or miticidal activity have been proposed. For practical use of such compounds as agrochemicals, it is required not only to have sufficiently high efficacy but to be less likely to cause chemical resistance, to cause neither phytotoxicity to plants nor soil pollution, and to be low toxic to livestock, fishes, etc.

Patent document 1 discloses a compound represented by the formula (A) or (B), etc.

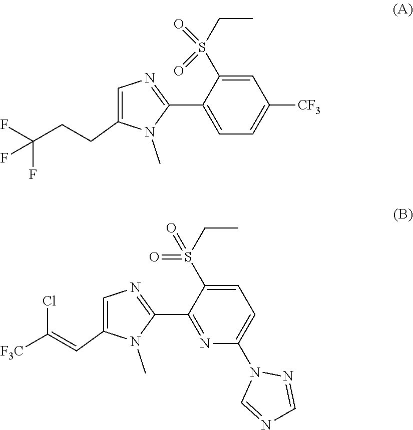 [SMILES: CCS(=O)(=O)c1cc(C(F)(F)F)ccc1-c1ncc(CCC(F)(F)F)n1C ; CCS(=O)(=O)c1ccc(-n2cncn2)nc1-c1ncc(/C=C(\Cl)C(F)(F)F)n1C]

Patent document 2 discloses a compound represented by the formula (C), etc.

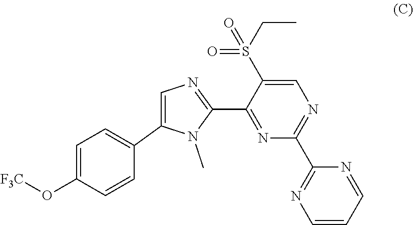 [SMILES: CCS(=O)(=O)c1cnc(-c2ncccn2)nc1-c1ncc(-c2ccc(OC(F)(F)F)cc2)n1C]

### PRIOR ART DOCUMENTS

### Patent Documents

Patent document 1: WO2017/104741A

Patent document 2: WO2018/052035A

### SUMMARY OF THE INVENTION

### Object to be Solved by the Invention

An object of the present invention is to provide a heteroaryl azole compound that is excellent in pest control activity, particularly, insecticidal activity and/or miticidal activity, is excellent in safety, and may be industrially advantageously synthesized. Another object of the present invention is to provide a pest control agent, an insecticide or miticide, an ectoparasite control agent, or an endoparasite control agent or expellant containing the heteroaryl azole compound as an active ingredient.

### Means to Solve the Object

As a result of diligent studies to attain the objects, the present invention including the following aspects has been completed.

[1] A compound represented by the formula (I), an N-oxide compound, stereoisomer, tautomer or hydrate thereof or a salt of any of these compounds:

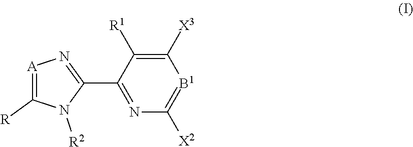

wherein

A represents CH or a nitrogen atom;

B¹ represents CX¹ or a nitrogen atom;

X¹, X² and X³ each independently represent a hydrogen atom, a substituted or unsubstituted C1-6 alkyl group, a substituted or unsubstituted C2-6 alkenyl group, a substituted or unsubstituted C2-6 alkynyl group, a hydroxy group, a substituted or unsubstituted C1-6 alkoxy group, a substituted or unsubstituted C1-6 alkoxycarbonyl group, a formyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-aryl group, a substituted or unsubstituted 3- to 6-membered heterocyclyl group, a substituted or unsubstituted C6-10 aryloxy group, a substituted or unsubstituted 5- or 6-membered heteroaryloxy group, a substituted or unsubstituted amino group, a substituted or unsubstituted aminocarbonyl group, a substituted or unsubstituted hydrazinyl group, a nitro group, a cyano group, a halogeno group, a group represented by the formula: —CR³═N—OR⁴, a group represented by the formula: —N═CHNR⁵R⁶, or a group represented by the formula: —N=S(O)_{q}R⁷R⁸;
R³ and R⁴ each independently represent a hydrogen atom, a C1-6 alkyl group, or a C1-6 haloalkyl group;
R⁵, R⁶, R⁷ and R⁸ each represent a C1-6 alkyl group;
q represents 0 or 1;

R¹ represents a substituted or unsubstituted C1-6 alkylthio group, a substituted or unsubstituted C1-6 alkylsulfinyl group, or a substituted or unsubstituted C1-6 alkylsulfonyl group;

R² represents a substituted or unsubstituted C1-6 alkyl group; and

R represents a substituted or unsubstituted C1-6 alkyl group.

[2] The compound according to the above [1], an N-oxide compound, stereoisomer, tautomer or hydrate thereof or a salt of any of these compounds, wherein the formula (I) is the formula (II):

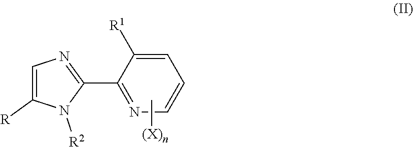

wherein

R¹, R², and R represent the same meanings as described in the formula (I);

X represents a substituted or unsubstituted C1-6 alkyl group, a substituted or unsubstituted C2-6 alkenyl group, a substituted or unsubstituted C2-6 alkynyl group, a hydroxy group, a substituted or unsubstituted C1-6 alkoxy group, a substituted or unsubstituted C1-6 alkoxycarbonyl group, a formyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-10 aryl group, a substituted or unsubstituted 3- to 6-membered heterocyclyl group, a substituted or unsubstituted C6-10 aryloxy group, a substituted or unsubstituted 5- or 6-membered heteroaryloxy group, a substituted or unsubstituted amino group, a substituted or unsubstituted aminocarbonyl group, a substituted or unsubstituted hydrazinyl group, a nitro group, a cyano group, a halogeno group, a group represented by the formula: —CR³═N—OR⁴, a group represented by the formula: —N═CHNR⁵R⁶, or a group represented by the formula: —N═S(O)_{q}R⁷R⁸;
R³ and R⁴ each independently represent a hydrogen atom, a C1-6 alkyl group, or a C1-6 haloalkyl group;
R⁵, R⁶, R⁷ and R⁸ each represent a C1-6 alkyl group;
q represents 0 or 1; and

n represents a chemically acceptable number of X and is any integer of 0 to 3, and when n is 2 or larger, each of the X is the same or different.

[3] The compound according to the above [1], an N-oxide compound, stereoisomer, tautomer or hydrate thereof or a salt of any of these compounds, wherein the formula (I) is the formula (III):

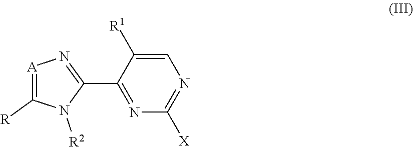

wherein

A, R¹, R², and R have the same meanings as described in the formula (I); and

X has the same meaning as described in the formula (II).

[4] The compound according to the above [1], an N-oxide compound, stereoisomer, tautomer or hydrate thereof or a salt of any of these compounds, wherein in the formula (I), R represents a C1-6 haloalkyl group.
[5] A pest control agent comprising at least one active ingredient selected from the group consisting of a compound according to any one of the above [1] to [4], an N-oxide compound, stereoisomer, tautomer and hydrate thereof and a salt of any of these compounds.
[6] An insecticide or miticide comprising at least one active ingredient selected from the group consisting of a compound according to any one of the above [1] to [4], an N-oxide compound, stereoisomer, tautomer and hydrate thereof and a salt of any of these compounds.
[7] An ectoparasite control agent comprising at least one active ingredient selected from the group consisting of a compound according to any one of the above [1] to [4], an N-oxide compound, stereoisomer, tautomer and hydrate thereof and a salt of any of these compounds.
[8] An endoparasite control agent or expellant comprising at least one active ingredient selected from the group consisting of a compound according to any one of the above [1] to [4], an N-oxide compound, stereoisomer, tautomer and hydrate thereof and a salt of any of these compounds.
[9] A seed treatment agent or vegetative propagation organ treatment agent comprising at least one active ingredient selected from the group consisting of a compound according to any one of the above [1] to [4], an N-oxide compound, stereoisomer, tautomer and hydrate thereof and a salt of any of these compounds.
[10] A granular agrochemical composition for paddy rice seedling nursery box treatment comprising at least one active ingredient selected from the group consisting of a compound according to any one of the above [1] to [4], an N-oxide compound, stereoisomer, tautomer and hydrate thereof and a salt of any of these compounds.
[11] A soil treatment agent comprising at least one active ingredient selected from the group consisting of a compound according to any one of the above [1] to [4], an N-oxide compound, stereoisomer, tautomer and hydrate thereof and a salt of any of these compounds.
[12] A bait agent comprising at least one active ingredient selected from the group consisting of a compound according to any one of the above [1] to [4], an N-oxide compound, stereoisomer, tautomer and hydrate thereof and a salt of any of these compounds.
[13] A plant growth promoter comprising at least one active ingredient selected from the group consisting of a compound according to any one of the above [1] to [4], an N-oxide compound, stereoisomer, tautomer and hydrate thereof and a salt of any of these compounds.

### Effect of the Invention

The heteroaryl azole compound of the present invention may control pests that are problems associated with crops or hygiene. Particularly, the heteroaryl azole compound of the present invention may effectively control agricultural insect pests and mites at a lower concentration. Furthermore, the heteroaryl azole compound of the present invention may effectively control ectoparasites and endoparasites harmful to humans and animals.

### MODE OF CARRYING OUT THE INVENTION

### [Heteroaryl Azole Compound]

The heteroaryl azole compound of the present invention is a compound represented by the formula (I) (hereinafter, also referred to as compound (I)), an N-oxide compound, stereoisomer, tautomer or hydrate thereof or a salt of any of these compounds. The compound represented by the formula (I) includes every stereoisomer which is an enantiomer or a diastereomer.

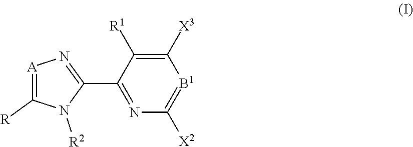

In the present invention, the term “unsubstituted” means a group consisting of only a mother nucleus. Only the name of a group consisting of a mother nucleus without the term “substituted” means an “unsubstituted” group unless otherwise specified.

On the other hand, the term “substituted” means that any hydrogen atom of a group consisting of a mother nucleus is substituted with a group (substituent) having a structure that is the same as or different from that of the mother nucleus. Thus, the “substituent” means another group bonded to the group consisting of a mother nucleus. The number of the substituent may be one or more. Two or more substituents are the same or different.

Terms such as “C1-6” mean that the number of carbon atoms in the group consisting of a mother nucleus is 1 to 6, etc. This number of carbon atoms does not include the number of carbon atoms present in the substituent. For example, a butyl group having an ethoxy group as a substituent is classified into a C2 alkoxy C4 alkyl group.

The “substituent” is not particularly limited as long as the substituent is chemically acceptable and produces the effect of the present invention. Hereinafter, a group capable of serving as the “substituent” is exemplified:

a C1-6 alkyl group such as a methyl group, an ethyl group, a n-propyl group, an i-propyl group, a n-butyl group, a s-butyl group, an i-butyl group, a t-butyl group, a n-pentyl group, and a n-hexyl group;

a C2-6 alkenyl group such as a vinyl group, a 1-propenyl group, a 2-propenyl group (allyl group), a 1-butenyl group, a 2-butenyl group, a 3-butenyl group, a 1-methyl-2-propenyl group, and a 2-methyl-2-propenyl group;

a C2-6 alkynyl group such as an ethynyl group, a 1-propynyl group, a 2-propynyl group, a 1-butynyl group, a 2-butynyl group, a 3-butynyl group, and a 1-methyl-2-propynyl group;

a C3-8 cycloalkyl group such as a cyclopropyl group, a cyclobutyl group, a cyclopentyl group, a cyclohexyl group, and a cubanyl group;

a C6-10 aryl group such as a phenyl group and a naphthyl group;

a C6-10 aryl C1-6 alkyl group such as a benzyl group and a phenethyl group;

a 3- to 6-membered heterocyclyl group;

a 3- to 6-membered heterocyclyl C1-6 alkyl group;

a hydroxy group;

a C1-6 alkoxy group such as a methoxy group, an ethoxy group, a n-propoxy group, an i-propoxy group, a n-butoxy group, a s-butoxy group, an i-butoxy group, and a t-butoxy group;

a C2-6 alkenyloxy group such as a vinyloxy group, an allyloxy group, a propenyloxy group, and a butenyloxy group;

a C2-6 alkynyloxy group such as an ethynyloxy group and a propargyloxy group;

a C6-10 aryloxy group such as a phenoxy group and a naphthoxy group;

a C6-10 aryl C1-6 alkoxy group such as a benzyloxy group and a phenethyloxy group;

a 5- or 6-membered heteroaryloxy group such as a thiazolyloxy group and a pyridyloxy group;

a 5- or 6-membered heteroaryl C1-6 alkyloxy group such as a thiazolylmethyloxy group and a pyridylmethyloxy group;

a formyl group;

a C1-6 alkylcarbonyl group such as an acetyl group and a propionyl group;

a formyloxy group;

a C1-6 alkylcarbonyloxy group such as an acetyloxy group and a propionyloxy group;

a C6-10 arylcarbonyl group such as a benzoyl group;

a C1-6 alkoxycarbonyl group such as a methoxycarbonyl group, an ethoxycarbonyl group, a n-propoxycarbonyl group, an i-propoxycarbonyl group, a n-butoxycarbonyl group, and a t-butoxycarbonyl group;

a C1-6 alkoxycarbonyloxy group such as a methoxycarbonyloxy group, an ethoxycarbonyloxy group, a n-propoxycarbonyloxy group, an i-propoxycarbonyloxy group, a n-butoxycarbonyloxy group, and a t-butoxycarbonyloxy group;

a carboxy group;

a halogeno group such as a fluoro group, a chloro group, a bromo group, and an iodo group;

a C1-6 haloalkyl group such as a fluoromethyl group, a difluoromethyl group, a trifluoromethyl group, a 2,2,2-trifluoroethyl group, a pentafluoroethyl group, a 3,3,3-trifluoropropyl group, a 2,2,3,3,3-pentafluoropropyl group, a perfluoropropyl group, a 2,2,2-trifluoro-1-trifluoromethylethyl group, a perfluoroisopropyl group, a 4-fluorobutyl group, a 2,2,3,3,4,4,4-heptafluorobutyl group, a perfluorobutyl group, a perfluoropentyl group, a perfluorohexyl group, a chloromethyl group, a bromomethyl group, a dichloromethyl group, a dibromomethyl group, a trichloromethyl group, a tribromomethyl group, a 1-chloroethyl group, a 2,2,2-trichloroethyl group, a 4-chlorobutyl group, a perchlorohexyl group, and a 2,4,6-trichlorohexyl group;

a C2-6 haloalkenyl group such as a 2-chloro-1-propenyl group and a 2-fluoro-1-butenyl group;

a C2-6 haloalkynyl group such as a 4,4-dichloro-1-butynyl group, a 4-fluoro-1-pentynyl group, and a 5-bromo-2-pentynyl group;

a C1-6 haloalkoxy group such as a trifluoromethoxy group, a 2-chloro-n-propoxy group, and a 2,3-dichlorobutoxy group;

a C2-6 haloalkenyloxy group such as a 2-chloropropenyloxy group and a 3-bromobutenyloxy group; a C1-6 haloalkylcarbonyl group such as a chloroacetyl group, a trifluoroacetyl group, and a trichloroacetyl group;

an amino group;

a C1-6 alkyl-substituted amino group such as a methylamino group, a dimethylamino group, and a diethylamino group;

a C6-10 arylamino group such as an anilino group and a naphthylamino group;

a C6-10 aryl C1-6 alkylamino group such as a benzylamino group and a phenethylamino group;

a formylamino group;

a C1-6 alkylcarbonylamino group such as an acetylamino group, a propanoylamino group, a butyrylamino group, and an i-propylcarbonylamino group;

a C1-6 alkoxycarbonylamino group such as a methoxycarbonylamino group, an ethoxycarbonylamino group, a n-propoxycarbonylamino group, and an i-propoxycarbonylamino group;

an unsubstituted or substituted aminocarbonyl group such as an aminocarbonyl group, a dimethylaminocarbonyl group, a phenylaminocarbonyl group, and a N-phenyl-N-methylaminocarbonyl group;

an imino C1-6 alkyl group such as an iminomethyl group, a (1-imino)ethyl group, and a (1-imino)-n-propyl group;

a substituted or unsubstituted N-hydroxyimino C1-6 alkyl group such as a N-hydroxy-iminomethyl group, a (1-(N-hydroxy)-imino)ethyl group, a (1-(N-hydroxy)-imino)propyl group, a N-methoxy-iminomethyl group, and a (1-(N-methoxy)-imino)ethyl group;

a C1-6 alkoxyimino group such as a methoxyimino group, an ethoxyimino group, a n-propoxyimino group, an i-propoxyimino group, and a n-butoxyimino group;

an aminocarbonyloxy group;

a C1-6 alkyl-substituted aminocarbonyloxy group such as an ethylaminocarbonyloxy group and a dimethylaminocarbonyloxy group;

a mercapto group;

a C1-6 alkylthio group such as a methylthio group, an ethylthio group, a n-propylthio group, an i-propylthio group, a n-butylthio group, an i-butylthio group, a s-butylthio group, and a t-butylthio group;

a C1-6 haloalkylthio group such as a trifluoromethylthio group and a 2,2,2-trifluoroethylthio group;

a C6-10 arylthio group such as a phenylthio group and a naphthylthio group;

a 5- or 6-membered heteroarylthio group such as a thiazolylthio group and a pyridylthio group;

a C1-6 alkylsulfinyl group such as a methylsulfinyl group, an ethylsulfinyl group, and a t-butylsulfinyl group;

a C1-6 haloalkylsulfinyl group such as a trifluoromethylsulfinyl group and a 2,2,2-trifluoroethylsulfinyl group;

a C6-10 arylsulfinyl group such as a phenylsulfinyl group;

a 5- or 6-membered heteroarylsulfinyl group such as a thiazolylsulfinyl group and a pyridylsulfinyl group;

a C1-6 alkylsulfonyl group such as a methylsulfonyl group, an ethylsulfonyl group, and a t-butylsulfonyl group;

a C1-6 haloalkylsulfonyl group such as a trifluoromethylsulfonyl group and a 2,2,2-trifluoroethylsulfonyl group;

a C6-10 arylsulfonyl group such as a phenylsulfonyl group;

a 5- or 6-membered heteroarylsulfonyl group such as a thiazolylsulfonyl group and a pyridylsulfonyl group; a C1-6 alkylsulfonyloxy group such as a methylsulfonyloxy group, an ethylsulfonyloxy group, and a t-butylsulfonyloxy group;

a C1-6 haloalkylsulfonyloxy group such as a trifluoromethylsulfonyloxy group and a 2,2,2-trifluoroethylsulfonyloxy group;

a tri-C1-6 alkyl-substituted silyl group such as a trimethylsilyl group, a triethylsilyl group, and a t-butyldimethylsilyl group;

a tri-C6-10 aryl-substituted silyl group such as a triphenylsilyl group;

a cyano group; and a nitro group.

For these “substituents”, any hydrogen atom in each substituent may be substituted with a group having a distinct structure. In this case, as the “substituent”, a C1-6 alkyl group, a C1-6 haloalkyl group, a C1-6 alkoxy group, a C1-6 haloalkoxy group, a halogeno group, a cyano group, a nitro group or the like may be exemplified.

The “3- to 6-membered heterocyclyl group” described above contains 1 to 4 heteroatoms selected from the group consisting of a nitrogen atom, an oxygen atom and a sulfur atom as ring-constituting atoms. The heterocyclyl group may be either monocyclic or polycyclic. The polycyclic heterocyclyl group has at least one hetero ring, and the remaining ring(s) may be any of a saturated alicyclic ring, an unsaturated alicyclic ring and an aromatic ring. As the “3- to 6-membered heterocyclyl group”, a 3- to 6-membered saturated heterocyclyl group, a 5- or 6-membered heteroaryl group, a 5- or 6-membered partially unsaturated heterocyclyl group or the like may be exemplified.

As the 3- to 6-membered saturated heterocyclyl group, an aziridinyl group, an epoxy group, a pyrrolidinyl group, a tetrahydrofuranyl group, a thiazolidinyl group, a piperidyl group, a piperazinyl group, a morpholinyl group, a dioxolanyl group, a dioxanyl group or the like may be exemplified.

As the 5-membered heteroaryl group, a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, a tetrazolyl group or the like may be exemplified.

As the 6-membered heteroaryl group, a pyridyl group, a pyrazinyl group, a pyrimidinyl group, a pyridazinyl group, a triazinyl group or the like may be exemplified.

### [A]

In the formula (I), A represents CH or a nitrogen atom.

Specifically, the compound represented by the formula (I) is a compound represented by the formula (I-1) or the formula (I-2).

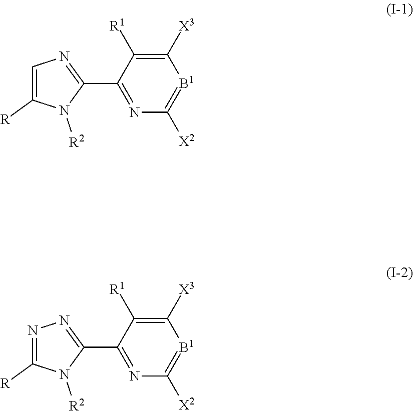

In the formula (I-1) and the formula (I-2), B¹, R¹, R², R, X² and X³ have the same meanings as described in the formula (I).

### [B¹]

In the formula (I), B¹ represents CX¹ or a nitrogen atom.

Specifically, the compound represented by the formula (I) is a compound represented by the formula (I-3) or the formula (I-4).

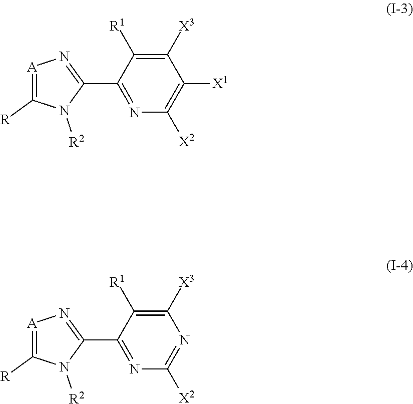

In the formula (1-3) and the formula (1-4), A, R¹, R², R, X² and X³ have the same meanings as described in the formula (I).

In the formula (I-3), X¹ represents a hydrogen atom, a substituted or unsubstituted C1-6 alkyl group, a substituted or unsubstituted C2-6 alkenyl group, a substituted or unsubstituted C2-6 alkynyl group, a hydroxy group, a substituted or unsubstituted C1-6 alkoxy group, a substituted or unsubstituted C1-6 alkoxycarbonyl group, a formyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-aryl group, a substituted or unsubstituted 3- to 6-membered heterocyclyl group, a substituted or unsubstituted C6-10 aryloxy group, a substituted or unsubstituted 5- or 6-membered heteroaryloxy group, a substituted or unsubstituted amino group, a substituted or unsubstituted aminocarbonyl group, a substituted or unsubstituted hydrazinyl group, a nitro group, a cyano group, a halogeno group, a group represented by the formula: —CR³═N—OR⁴, a group represented by the formula: —N═CHNR⁵R⁶, or a group represented by the formula: —N═S(O)_{q}R⁷R⁸. R³ and R⁴ each independently represent a hydrogen atom, a C1-6 alkyl group, or a C1-6 haloalkyl group. R⁵, R⁶, R⁷ and R⁸ each represent a C1-6 alkyl group. q represents 0 or 1.

The “C1-6 alkyl group” in X¹ may be linear or branched. As the “C1-6 alkyl group”, a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, an i-hexyl group or the like may be exemplified.

As the substituent on the “C1-6 alkyl group” in X¹, a halogeno group such as a fluoro group, a chloro group, a bromo group, and an iodo group; a cyano group; an alkoxycarbonyl group such as an ethoxycarbonyl group, or the like may be preferably exemplified.

As the “C2-6 alkenyl group” in X¹, a vinyl group, a 1-propenyl group, a 2-propenyl group, a 1-butenyl group, a 2-butenyl group, a 3-butenyl group, a 1-methyl-2-propenyl group, a 2-methyl-2-propenyl group, a 1-pentenyl group, a 2-pentenyl group, a 3-pentenyl group, a 4-pentenyl group, a 1-methyl-2-butenyl group, a 2-methyl-2-butenyl group, a 1-hexenyl group, a 2-hexenyl group, a 3-hexenyl group, a 4-hexenyl group, a 5-hexenyl group or the like may be exemplified.

As the “C2-6 alkynyl group” in X¹, an ethynyl group, a 1-propynyl group, a 2-propynyl group, a 1-butynyl group, a 2-butynyl group, a 3-butynyl group, a 1-methyl-2-propynyl group, a 2-methyl-3-butynyl group, a 1-pentynyl group, a 2-pentynyl group, a 3-pentynyl group, a 4-pentynyl group, a 1-methyl-2-butynyl group, a 2-methyl-3-pentynyl group, a 1-hexynyl group, a 1,1-dimethyl-2-butynyl group or the like may be exemplified.

As the “C1-6 alkoxy group” in X¹, a methoxy group, an ethoxy group, a n-propoxy group, an i-propoxy group, a n-butoxy group, a s-butoxy group, an i-butoxy group, a t-butoxy group or the like may be exemplified.

As the “C1-6 alkoxycarbonyl group” in X¹, a methoxycarbonyl group, an ethoxycarbonyl group, a n-propoxycarbonyl group, an i-propoxycarbonyl group, a n-butoxycarbonyl group, a t-butoxycarbonyl group or the like may be exemplified.

As the “C1-6 alkylthio group” in X¹, a methylthio group, an ethylthio group, a n-propylthio group, a n-butylthio group, a n-pentylthio group, a n-hexylthio group, an i-propylthio group, an i-butylthio group or the like may be exemplified.

As the “C1-6 alkylsulfinyl group” in X¹, a methylsulfinyl group, an ethylsulfinyl group, a t-butylsulfinyl group or the like may be exemplified.

As the “C1-6 alkylsulfonyl group” in X¹, a methylsulfonyl group, an ethylsulfonyl group, a t-butylsulfonyl group or the like may be exemplified.

As the substituents on the “C2-6 alkenyl group”, the “C2-6 alkynyl group”, the “C1-6 alkoxy group”, the “C1-6 alkylthio group”, the “C1-6 alkylsulfinyl group” and the “C1-6 alkylsulfonyl group” in X¹, a halogeno group such as a fluoro group, a chloro group, a bromo group, and an iodo group; and a C1-6 alkoxy group such as a methoxy group, an ethoxy group, a n-propoxy group, an i-propoxy group, a n-butoxy group, a s-butoxy group, an i-butoxy group, and a t-butoxy group may be preferably exemplified.

As the “C3-8 cycloalkyl group” in X¹, a cyclopropyl group, a cyclobutyl group, a cyclopentyl group, a cyclohexyl group, a cubanyl group or the like may be exemplified.

As the “C6-10 aryl group” in X¹, a phenyl group, a naphthyl group, an indenyl group, an indanyl group, a tetralinyl group or the like may be exemplified.

The “3- to 6-membered heterocyclyl group” in X¹ contains 1 to 4 heteroatoms selected from the group consisting of a nitrogen atom, an oxygen atom and a sulfur atom as ring-constituting atoms. The heterocyclyl group may be either monocyclic or polycyclic. The polycyclic heterocyclyl group has at least one hetero ring, and the remaining ring(s) may be any of a saturated alicyclic ring, an unsaturated alicyclic ring and an aromatic ring. As the “3- to 6-membered heterocyclyl group”, a 3- to 6-membered saturated heterocyclyl group, a 5- or 6-membered heteroaryl group, a 5- or 6-membered partially unsaturated heterocyclyl group or the like may be exemplified.

As the 3- to 6-membered saturated heterocyclyl group, an aziridinyl group, an epoxy group, a pyrrolidinyl group, a tetrahydrofuranyl group, a thiazolidinyl group, a piperidyl group, a piperazinyl group, a morpholinyl group, a dioxolanyl group, a dioxanyl group or the like may be exemplified.

As the 5- or 6-membered partially unsaturated heterocyclyl group, a 2-oxopyridin-1(2H)-yl group or the like may be exemplified.

As the 5-membered heteroaryl group, a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, a tetrazolyl group or the like may be exemplified.

As the 6-membered heteroaryl group, a pyridyl group, a pyrazinyl group, a pyrimidinyl group, a pyridazinyl group, a triazinyl group or the like may be exemplified.

As the “C6-10 aryloxy group” in X¹, a phenoxy group, a naphthyloxy group or the like may be exemplified.

As the “5- or 6-membered heteroaryloxy group” in X¹, a pyridyloxy group, a pyrimidyloxy group or the like may be exemplified.

As the substituents on the “C3-8 cycloalkyl group”, the “C6-10 aryl group”, the “3- to 6-membered heterocyclyl group”, the “C6-10 aryloxy group”, and the “5- or 6-membered heteroaryloxy group” in X¹, a halogeno group such as a fluoro group, a chloro group, a bromo group, and an iodo group; a C1-6 alkyl group such as a methyl group, an ethyl group, a n-propyl group, an i-propyl group, a n-butyl group, a s-butyl group, an i-butyl group, a t-butyl group, a n-pentyl group, and a n-hexyl group; a C1-6 haloalkyl group such as a fluoromethyl group, a difluoromethyl group, a trifluoromethyl group, a 2,2,2-trifluoroethyl group, a pentafluoroethyl group, a 3,3,3-trifluoropropyl group, a 2,2,3,3,3-pentafluoropropyl group, a perfluoropropyl group, a 2,2,2-trifluoro-1-trifluoromethylethyl group, a perfluoroisopropyl group, a 4-fluorobutyl group, a 2,2,3,3,4,4,4-heptafluorobutyl group, a perfluorobutyl group, a perfluoropentyl group, a perfluorohexyl group, a chloromethyl group, a bromomethyl group, a dichloromethyl group, a dibromomethyl group, a trichloromethyl group, a tribromomethyl group, a 1-chloroethyl group, a 2,2,2-trichloroethyl group, a 4-chlorobutyl group, a perchlorohexyl group, and a 2,4,6-trichlorohexyl group; a C1-6 alkoxy group such as a methoxy group, an ethoxy group, a n-propoxy group, an i-propoxy group, a n-butoxy group, a s-butoxy group, an i-butoxy group, and a t-butoxy group; a C1-6 haloalkoxy group such as a 2-chloro-n-propoxy group, a 2,3-dichlorobutoxy group, and a trifluoromethoxy group; and an amino group may be preferably exemplified.

The “substituted or unsubstituted amino group” in X¹ is a group represented by “—NR^{a}R^{b}” or the like. In the formula, R^{a} and R^{b} each independently represent a hydrogen atom, a C1-6 alkyl group, a C3-8 cycloalkyl group, a formyl group, a C1-6 alkylcarbonyl group, a C3-8 cycloalkylcarbonyl group, or a substituted or unsubstituted aminocarbonyl group.

As the “C1-6 alkyl group” in R^{a} and R^{b}, the same group as listed in the above X¹ is exemplified.

As the “C3-8 cycloalkyl group” in R^{a} and R^{b}, the same group as listed in the above X¹ is exemplified.

As the “C1-6 alkylcarbonyl group” in R^{a} and R^{b}, an acetyl group, a propionyl group or the like may be exemplified.

As the “C3-8 cycloalkylcarbonyl group” in R^{a} and R^{b}, a cyclopropylcarbonyl group, a cyclobutylcarbonyl group, a cyclopentylcarbonyl group or the like may be exemplified.

As the “substituted or unsubstituted aminocarbonyl group” in R^{a} and R^{b}, an aminocarbonyl group, a methylaminocarbonyl group, an ethylaminocarbonyl group, a dimethylaminocarbonyl group or the like may be exemplified.

As the “substituted or unsubstituted aminocarbonyl group” in X¹, the same group as listed in the above R^{a} and R^{b} is exemplified.

The “substituted or unsubstituted hydrazinyl group” in X¹ is a group represented by the formula (a) or the like.

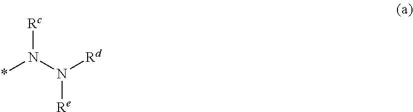

In the formula (a), * is a bonding position, and R^{c}, R^{d} and R^{e} each independently represent a hydrogen atom, a C1-6 alkyl group, or a substituted or unsubstituted phenylsulfonyl group or the like.

As the “C1-6 alkyl group” in R^{c}, R^{d} and R^{e}, the same group as listed in the above X¹ is exemplified.

As the “substituted phenylsulfonyl group” in R^{c}, R^{d} and R^{e}, a p-toluenesulfonyl group or the like may be exemplified.

As the “halogeno group” in X¹, a fluoro group, a chloro group, a bromo group, an iodo group or the like may be exemplified.

As the “C1-6 alkyl group” in R³, R⁴, R⁵, R⁶, R⁷ and R⁸ of the “group represented by the formula: —CR³═N—OR⁴”, the “group represented by the formula: —N═CHNR⁵R⁶” and the “group represented by the formula: —N═S(O)_{q}R⁷R⁸” in X¹, the same group as listed in the above X¹ is exemplified.

As the “C1-6 haloalkyl group” in R³ and R⁴, a C1-6 haloalkyl group such as a fluoromethyl group, a difluoromethyl group, a trifluoromethyl group, a 2,2,2-trifluoroethyl group, a pentafluoroethyl group, a 3,3,3-trifluoropropyl group, a 2,2,3,3,3-pentafluoropropyl group, a perfluoropropyl group, a 2,2,2-trifluoro-1-trifluoromethylethyl group, a perfluoroisopropyl group, a 4-fluorobutyl group, a 2,2,3,3,4,4,4-heptafluorobutyl group, a perfluorobutyl group, a perfluoropentyl group, a perfluorohexyl group, a chloromethyl group, a bromomethyl group, a dichloromethyl group, a dibromomethyl group, a trichloromethyl group, a tribromomethyl group, a 1-chloroethyl group, a 2,2,2-trichloroethyl group, a 4-chlorobutyl group, a perchlorohexyl group, a 2,4,6-trichlorohexyl group, or the like may be exemplified.

Specifically, the compound represented by the formula (I) is a compound represented by the formula (I-5), the formula (I-6), the formula (I-7) or the formula (I-8).

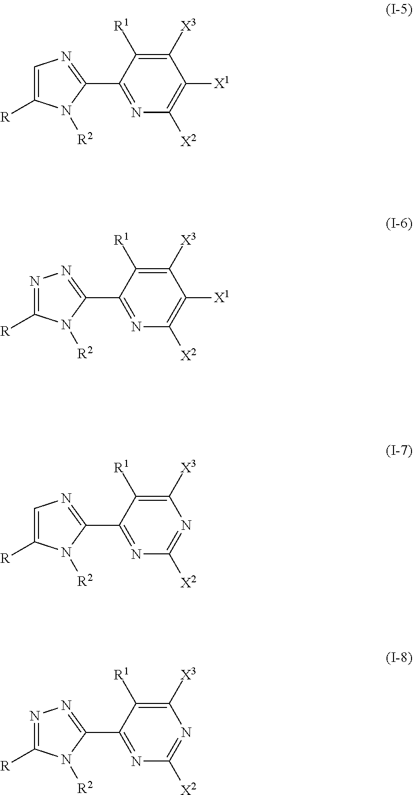

In the formula (I-5), the formula (I-6), the formula (1-7) and the formula (I-8), R¹, R², R, X² and X³ have the same meanings as described in the formula (I).

In the formula (I-5) and the formula (I-6), X¹ has the same meaning as described in the formula (I-3).

[X² and X³]

X² and X³ each independently represent a hydrogen atom, a substituted or unsubstituted C1-6 alkyl group, a substituted or unsubstituted C2-6 alkenyl group, a substituted or unsubstituted C2-6 alkynyl group, a hydroxy group, a substituted or unsubstituted C1-6 alkoxy group, a substituted or unsubstituted C1-6 alkoxycarbonyl group, a formyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-aryl group, a substituted or unsubstituted 3- to 6-membered heterocyclyl group, a substituted or unsubstituted C6-10 aryloxy group, a substituted or unsubstituted 5- or 6-membered heteroaryloxy group, a substituted or unsubstituted amino group, a substituted or unsubstituted aminocarbonyl group, a substituted or unsubstituted hydrazinyl group, a nitro group, a cyano group, a halogeno group, a group represented by the formula: —CR³═N—OR⁴, a group represented by the formula: —N═CHNR⁵R⁶, or a group represented by the formula: —N═S(O)_{q}R⁷R⁸. R³ and R⁴ each independently represent a hydrogen atom, a C1-6 alkyl group, or a C1-6 haloalkyl group. R⁵, R⁶, R⁷ and R⁸ each represent a C1-6 alkyl group. q represents 0 or 1.

As the “substituted or unsubstituted C1-6 alkyl group”, the “substituted or unsubstituted C2-6 alkenyl group”, the “substituted or unsubstituted C2-6 alkynyl group”, the “substituted or unsubstituted C1-6 alkoxy group”, the “substituted or unsubstituted C1-6 alkoxycarbonyl group”, the “substituted or unsubstituted C1-6 alkylthio group”, the “substituted or unsubstituted C1-6 alkylsulfinyl group”, the “substituted or unsubstituted C1-6 alkylsulfonyl group”, the “substituted or unsubstituted C3-8 cycloalkyl group”, the “substituted or unsubstituted C6-10 aryl group”, the “substituted or unsubstituted 3- to 6-membered heterocyclyl group”, the “substituted or unsubstituted C6-10 aryloxy group”, the “substituted or unsubstituted 5- or 6-membered heteroaryloxy group”, the “substituted or unsubstituted amino group”, the “substituted or unsubstituted aminocarbonyl group”, the “substituted or unsubstituted hydrazinyl group” and the “halogeno group” in X² and X³, the same group as listed in the above X¹ is exemplified.

The “C1-6 alkyl group” and the “C1-6 haloalkyl group” in R³ and R⁴ in the formula: —CR³═N—OR⁴ have the same meanings as already described.

The “C1-6 alkyl group” in R⁵ and R⁶ of the formula: —N═CHNR⁵R⁶ has the same meaning as already described.

The “C1-6 alkyl group” in R⁷ and R⁸ of the formula: —N═S(O)_{q}R⁷R⁸ has the same meaning as already described.

### [R¹]

In the formula (I), R¹ represents a substituted or unsubstituted C1-6 alkylthio group, a substituted or unsubstituted C1-6 alkylsulfinyl group, or a substituted or unsubstituted C1-6 alkylsulfonyl group.

As the “C1-6 alkylthio group”, the “C1-6 alkylsulfinyl group” and the “C1-6 alkylsulfonyl group” in R¹, the same group as listed in the above X¹ is exemplified.

As the substituents on the “C1-6 alkylthio group”, the “C1-6 alkylsulfinyl group” and the “C1-6 alkylsulfonyl group” in R¹, a halogeno group such as a fluoro group, a chloro group, a bromo group, and an iodo group may be preferably exemplified.

### [R²]

In the formula (I), R² represents a substituted or unsubstituted C1-6 alkyl group.

As the “C1-6 alkyl group” in R², the same group as listed in the above X¹ is exemplified.

As the substituent on the “C1-6 alkyl group” represented by R², a halogeno group such as a fluoro group, a chloro group, a bromo group, and an iodo group may be preferably exemplified.

### [R]

In the formula (I), R represents a substituted or unsubstituted C1-6 alkyl group.

As the “C1-6 alkyl group” in R, the same group as listed in the above X¹ is exemplified.

As the “substituted C1-6 alkyl group” in R, a C1-6 haloalkyl group such as a fluoromethyl group, a difluoromethyl group, a trifluoromethyl group, a 2,2,2-trifluoroethyl group, a pentafluoroethyl group, a 3,3,3-trifluoropropyl group, a 2,2,3,3,3-pentafluoropropyl group, a 1-chloro-2,2,3,3,3-pentafluoropropyl group, a 1,2,2,3,3,3-hexafluoropropyl group, a perfluoropropyl group, a 2,2,2-trifluoro-1-trifluoromethylethyl group, a perfluoroisopropyl group, a 4-fluorobutyl group, a 1,4,4,4-tetrafluorobutyl group, a 2,2,3,3,4,4,4-heptafluorobutyl group, a 1,2,2,3,3,4,4,4-octafluorobutyl group, a perfluorobutyl group, a 3,3,3-trifluoro-2-trifluoromethylpropyl group, a 1-chloro-2,3,3,3-tetrafluoro-2-trifluoromethylpropyl group, a 1,2,3,3,3-pentafluoro-2-trifluoromethylpropyl group, a 2,2,3,3,4,4,5,5,5-nonafluoropentyl group, a 1-chloro-2,2,3,3,4,4,5,5,5-nonafluoropentyl group, a 1,2,2,3,3,4,4,5,5,5-decafluoropentyl group, a perfluoropentyl group, a perfluorohexyl group, a chloromethyl group, a bromomethyl group, a dichloromethyl group, a dibromomethyl group, a trichloromethyl group, a tribromomethyl group, a 1-chloroethyl group, a 2,2,2-trichloroethyl group, a 4-chlorobutyl group, a perchlorohexyl group, and a 2,4,6-trichlorohexyl group; a hydroxy group-substituted C1-6 haloalkyl group such as a 1-hydroxy-2,2,3,3,4,4,5,5,5-nonafluoropentyl group; a C1-6 alkylcarbonyloxy group-substituted C1-6 haloalkyl group such as a 1-acetyloxy-2,2,3,3,4,4,5,5,5-nonafluoropentyl group; a C1-6 haloalkoxy C1-6 alkyl group such as a (2,2,3,3,3-pentafluoropropoxy)methyl group and a (2,2,3,3,4,4,4-heptafluorobutoxy)methyl group; and a C1-6 haloalkoxy C1-6 haloalkoxy C1-6 alkyl group such as a ((1,1,2-trifluoro-2-(trifluoromethoxy)ethoxy)methyl group may be exemplified.

As the substituent on the “C1-6 alkyl group” in R, a halogeno group such as a fluoro group, a chloro group, a bromo group, and an iodo group; a hydroxy group; a C1-6 haloalkoxy group such as a trifluoromethoxy group, a 2-chloro-n-propoxy group, a 2,3-dichlorobutoxy group, a 2,2,3,3,3-pentafluoropropoxy group, and a 2,2,3,3,4,4,4-heptafluorobutoxy group; and a C1-6 alkylcarbonyloxy group such as an acetyloxy group may be preferably exemplified.

### [Salt]

The salt of the compound (I) is not particularly limited as long as the salt is agriculturally or horticulturally acceptable. As the salt of the compound (I), for example, a salt of an inorganic acid such as hydrochloric acid and sulfuric acid; a salt of an organic acid such as acetic acid and lactic acid; a salt of an alkali metal such as lithium, sodium and potassium; a salt of an alkaline earth metal such as calcium and magnesium; a salt of a transition metal such as iron and copper; a salt of an organic base such as triethylamine, tributylamine, pyridine, and hydrazine; an ammonium salt, or the like may be exemplified.

### [Production Method]

The production method of the compound (I), the N-oxide compound, the stereoisomer, the tautomer, the hydrate or the salt of any of these compounds is not particularly limited. For example, these compounds may be obtained by known production methods described in Examples, etc. Alternatively, the N-oxide compound, salt, or the like of the compound (I) may be obtained by a known method from the compound (I).

### [Compound Represented by Formula (II)]

The compound represented by the formula (I) is preferably a compound represented by the formula (II).

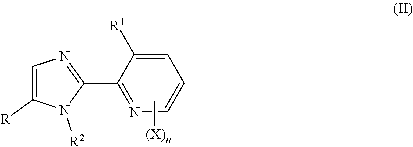

In the formula (II),

R¹, R², and R have the same meanings as described in the formula (I).

X represents a substituted or unsubstituted C1-6 alkyl group, a substituted or unsubstituted C2-6 alkenyl group, a substituted or unsubstituted C2-6 alkynyl group, a hydroxy group, a substituted or unsubstituted C1-6 alkoxy group, a substituted or unsubstituted C1-6 alkoxycarbonyl group, a formyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-10 aryl group, a substituted or unsubstituted 3- to 6-membered heterocyclyl group, a substituted or unsubstituted C6-10 aryloxy group, a substituted or unsubstituted 5- or 6-membered heteroaryloxy group, a substituted or unsubstituted amino group, a substituted or unsubstituted aminocarbonyl group, a substituted or unsubstituted hydrazinyl group, a nitro group, a cyano group, a halogeno group, a group represented by the formula: —CR³═N—OR⁴, a group represented by the formula: —N═CHNR⁵R⁶, or a group represented by the formula: —N═S(O)_{q}R⁷R⁸.

n represents a chemically acceptable number of X and is any integer of 0 to 3, and when n is 2 or larger, each of the X is the same or different.

As the “substituted or unsubstituted C1-6 alkyl group”, the “substituted or unsubstituted C2-6 alkenyl group”, the “substituted or unsubstituted C2-6 alkynyl group”, the “substituted or unsubstituted C1-6 alkoxy group”, the “substituted or unsubstituted C1-6 alkoxycarbonyl group”, the “substituted or unsubstituted C1-6 alkylthio group”, the “substituted or unsubstituted C1-6 alkylsulfinyl group”, the “substituted or unsubstituted C1-6 alkylsulfonyl group”, the “substituted or unsubstituted C3-8 cycloalkyl group”, the “substituted or unsubstituted C6-10 aryl group”, the “substituted or unsubstituted 3- to 6-membered heterocyclyl group”, the “substituted or unsubstituted C6-10 aryloxy group”, the “substituted or unsubstituted 5- or 6-membered heteroaryloxy group”, the “substituted or unsubstituted amino group”, the “substituted or unsubstituted aminocarbonyl group”, the “substituted or unsubstituted hydrazinyl group” and the “halogeno group” in X, the same group as listed in the above X¹ is exemplified.

The “C1-6 alkyl group” and the “C1-6 haloalkyl group” in R³ and R⁴ of the formula: —CR³═N—OR⁴ have the same meanings as already described.

The “C1-6 alkyl group” in R⁵ and R⁶ of the formula: —N═CHNR⁵R⁶ has the same meaning as already described.

The “C1-6 alkyl group” in R⁷ and R⁸ of the formula: —N═S(O)_{q}R⁷R⁸ has the same meaning as already described.

In the formula (II), R¹ is preferably a C1-6 alkylsulfonyl group, and R² is preferably a C1-6 alkyl group.

In the formula (II), R is preferably a C1-6 haloalkyl group such as a fluoromethyl group, a difluoromethyl group, a trifluoromethyl group, a 2,2,2-trifluoroethyl group, a pentafluoroethyl group, a 3,3,3-trifluoropropyl group, a 2,2,3,3,3-pentafluoropropyl group, a 1-chloro-2,2,3,3,3-pentafluoropropyl group, a 1,2,2,3,3,3-hexafluoropropyl group, a perfluoropropyl group, a 2,2,2-trifluoro-1-trifluoromethylethyl group, a perfluoroisopropyl group, a 4-fluorobutyl group, a 1,4,4,4-tetrafluorobutyl group, a 2,2,3,3,4,4,4-heptafluorobutyl group, a 1,2,2,3,3,4,4,4-octafluorobutyl group, a perfluorobutyl group, a 3,3,3-trifluoro-2-trifluoromethylpropyl group, a 1-chloro-2,3,3,3-tetrafluoro-2-trifluoromethylpropyl group, a 1,2,3,3,3-pentafluoro-2-trifluoromethylpropyl group, a 2,2,3,3,4,4,5,5,5-nonafluoropentyl group, a 1-chloro-2,2,3,3,4,4,5,5,5-nonafluoropentyl group, a 1,2,2,3,3,4,4,5,5,5-decafluoropentyl group, a perfluoropentyl group, a perfluorohexyl group, a chloromethyl group, a bromomethyl group, a dichloromethyl group, a dibromomethyl group, a trichloromethyl group, a tribromomethyl group, a 1-chloroethyl group, a 2,2,2-trichloroethyl group, a 4-chlorobutyl group, a perchlorohexyl group, or a 2,4,6-trichlorohexyl group, and more preferably a C1-6 fluoroalkyl group such as a difluoromethyl group, a trifluoromethyl group, a 2,2,2-trifluoroethyl group, a pentafluoroethyl group, a 3,3,3-trifluoropropyl group, a 2,2,3,3,3-pentafluoropropyl group, a 1,2,2,3,3,3-hexafluoropropyl group, a perfluoropropyl group, a 2,2,2-trifluoro-1-trifluoromethylethyl group, a perfluoroisopropyl group, a 4-fluorobutyl group, a 1,4,4,4-tetrafluorobutyl group, a 2,2,3,3,4,4,4-heptafluorobutyl group, a 1,2,2,3,3,4,4,4-octafluorobutyl group, a perfluorobutyl group, a 3,3,3-trifluoro-2-trifluoromethylpropyl group, a 1,2,3,3,3-pentafluoro-2-trifluoromethylpropyl group, a 2,2,3,3,4,4,5,5,5-nonafluoropentyl group, a 1,2,2,3,3,4,4,5,5,5-decafluoropentyl group, a perfluoropentyl group, or a perfluorohexyl group.

In the formula (II), X is preferably a halogeno group, a substituted or unsubstituted C1-6 alkyl group, a substituted or unsubstituted C1-6 alkoxy group, a formyl group, a C3-8 cycloalkyl group, a substituted or unsubstituted C6-10 aryl group, a substituted or unsubstituted 3- to 6-membered heterocyclyl group, a substituted or unsubstituted 5- or 6-membered heteroaryloxy group, a substituted or unsubstituted amino group, or a group represented by the formula: “—CR³═N—OR⁴”, more preferably a substituted or unsubstituted C1-6 alkyl group, a C3-8 cycloalkyl group, a substituted or unsubstituted phenyl group, or a substituted or unsubstituted 6-membered heteroaryl group, and particularly preferably a C3-8 cycloalkyl group, a substituted or unsubstituted phenyl group, or a substituted or unsubstituted 6-membered heteroaryl group.

In the formula (II), n is preferably 1.

### [Compound Represented by Formula (III)]

The compound represented by the formula (I) is preferably a compound represented by the formula (III).

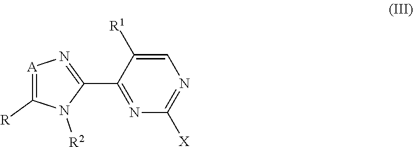

In the formula (III), A, R¹, R², and R have the same meanings as described in the formula (I).

X has the same meaning as described in the formula (II).

In the formula (III), R¹ is preferably a C1-6 alkylsulfonyl group. R² is preferably a C1-6 alkyl group. R is preferably a C1-6 fluoroalkyl group. X is preferably a substituted or unsubstituted 5- or 6-membered heteroaryl group, and more preferably a substituted or unsubstituted 5-membered heteroaryl group.

The compound represented by the formula (I) is preferably any of the following compounds.

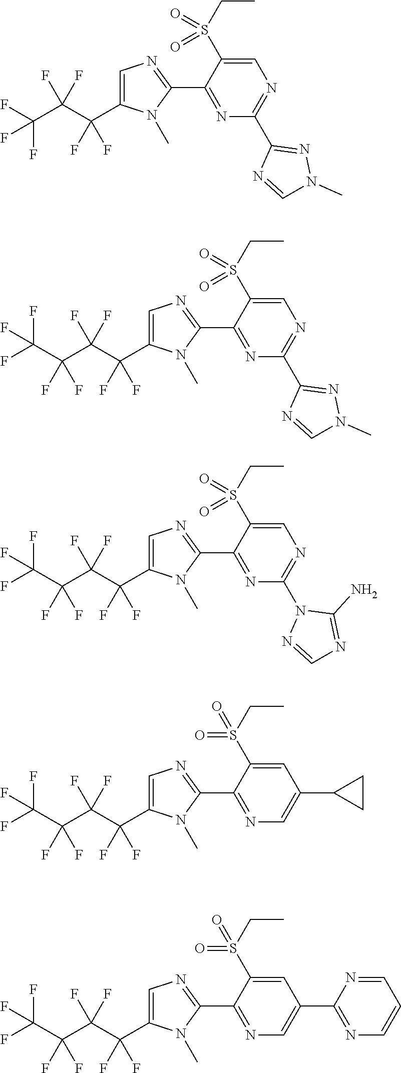

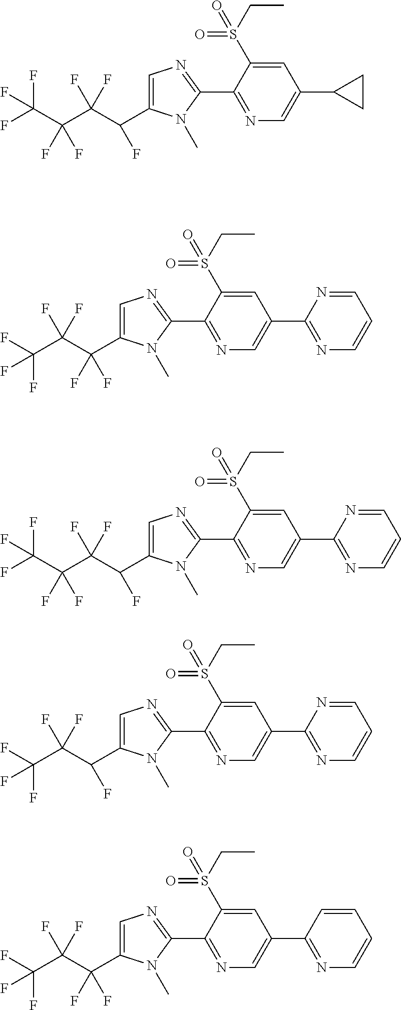 [SMILES: CCS(=O)(=O)c1cc(C2CC2)cnc1-c1ncc(C(F)C(F)(F)C(F)(F)C(F)(F)F)n1C ; CCS(=O)(=O)c1cc(-c2ncccn2)cnc1-c1ncc(C(F)(F)C(F)(F)C(F)(F)F)n1C ; CCS(=O)(=O)c1cc(-c2ncccn2)cnc1-c1ncc(C(F)C(F)(F)C(F)(F)C(F)(F)F)n1C ; CCS(=O)(=O)c1cc(-c2ncccn2)cnc1-c1ncc(C(F)C(F)(F)C(F)(F)F)n1C ; CCS(=O)(=O)c1cc(-c2ccccn2)cnc1-c1ncc(C(F)(F)C(F)(F)C(F)(F)F)n1C]

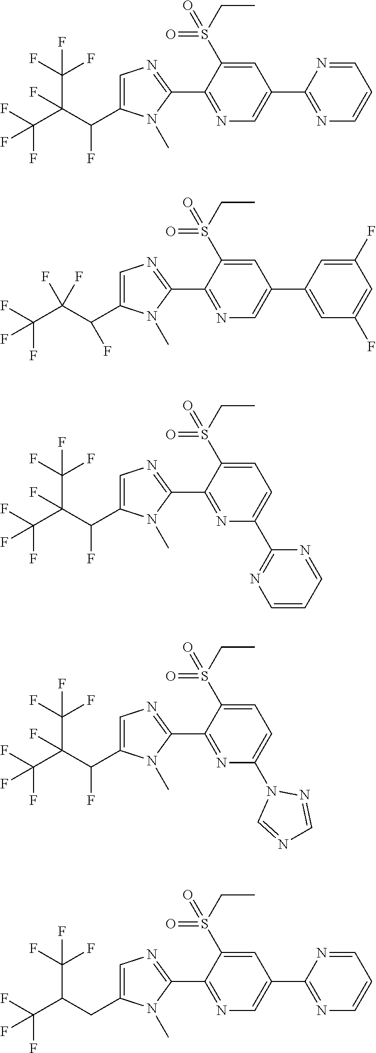

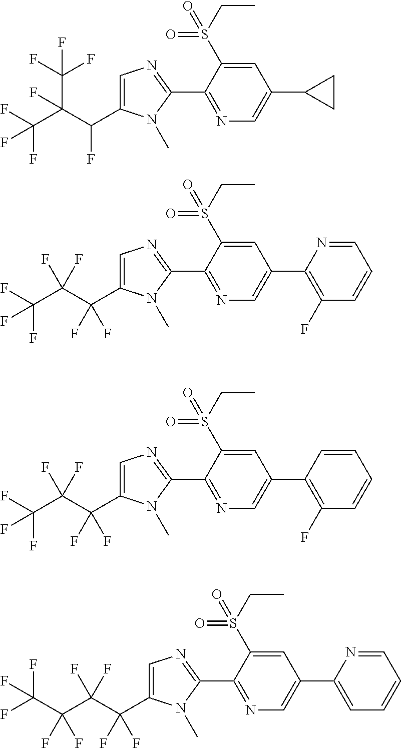

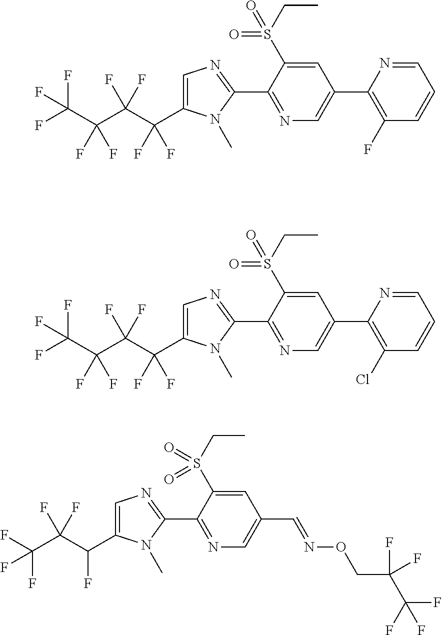 [SMILES: CCS(=O)(=O)c1cc(-c2ncccc2F)cnc1-c1ncc(C(F)(F)C(F)(F)C(F)(F)C(F)(F)F)n1C ; CCS(=O)(=O)c1cc(-c2ncccc2Cl)cnc1-c1ncc(C(F)(F)C(F)(F)C(F)(F)C(F)(F)F)n1C ; CCS(=O)(=O)c1cc(/C=N/OCC(F)(F)C(F)(F)F)cnc1-c1ncc(C(F)C(F)(F)C(F)(F)F)n1C]

The heteroaryl azole compound of the present invention is excellent in control effect on pests such as various agricultural insect pests and mites affecting the growth of plants.

Also, the heteroaryl azole compound of the present invention is a highly safe substance because of less phytotoxicity to crops and low toxicity to fishes and warm-blooded animals. Hence, the heteroaryl azole compound of the present invention is useful as an active ingredient for insecticides or miticides.

Furthermore, in recent years, many insect pests such as diamondback moth, white-backed plant hopper, leafhopper, and aphid have developed resistance to various existing chemicals, causing problems of insufficient efficacy of these chemicals. Thus, chemicals effective for insect pests of resistant strains have been desired. The heteroaryl azole compound of the present invention exhibits an excellent control effect not only on sensitive strains but also on insect pests of various resistant strains and even mites of miticide-resistant strains.

The heteroaryl azole compound of the present invention is excellent in control effect on ectoparasites and endoparasites harmful to humans and animals. Also, the heteroaryl azole compound of the present invention is a highly safe substance because of low toxicity to fishes and warm-blooded animals. Hence, the heteroaryl azole compound of the present invention is useful as an active ingredient for ectoparasite and endoparasite control agents.

The heteroaryl azole compound of the present invention exhibits efficacy at every developmental stage of organisms to be controlled, and exhibits an excellent control effect on, for example, eggs, nymphs, larvae, pupae, and adults of mites, insects, and the like.

### [Pest Control Agent]

The pest control agent of the present invention contains at least one active ingredient selected from the heteroaryl azole compounds of the present invention. The amount of the heteroaryl azole compound contained in the pest control agent of the present invention is not particularly limited as long as its pest control effect is exhibited. The pest control agent is an agent controlling pests and includes an insecticide or miticide, an ectoparasite control agent, or an endoparasite control agent or expellant, or the like.

### [Insecticide or Miticide]

The insecticide or miticide of the present invention contains at least one active ingredient selected from the heteroaryl azole compounds of the present invention. The amount of the heteroaryl azole compound contained in the insecticide or miticide of the present invention is not particularly limited as long as its insecticidal or miticidal effect is exhibited.

The pest control agent or the insecticide or miticide of the present invention is preferably used for plants such as cereals; vegetables; root vegetables; tubers and roots; flowers and ornamental plants; fruit trees; ornamental foliage plants and trees of tea, coffee, cacao, and the like; feed crops; lawn grasses; and cotton.

In the application to plants, the pest control agent or the insecticide or miticide of the present invention may be used for any site such as a leaf, a stem, a stalk, a flower, a bud, a fruit, a seed, a sprout, a root, a tuber, a tuberous root, a shoot, or a slip.

The pest control agent or the insecticide or miticide of the present invention is not particularly limited by the species of the plant to which the pest control agent or the insecticide or miticide is applied. As the plant species, for example, an original species, a variant species, an improved variety, a cultivar, a mutant, a hybrid, a genetically modified organism (GMO) or the like may be exemplified.

The pest control agent of the present invention may be used in seed treatment, foliage application, soil application, submerged application, or the like in order to control various agricultural insect pests and mites.

Specific examples of various agricultural insect pests and mites controllable with the pest control agent of the present invention will be shown below.

### (1) Butterflies or Moths of the Order Lepidoptera

(a) moths of the family Arctiidae, for example, Hyphantria cunea and Lemyra imparilis;
(b) moths of the family Bucculatricidae, for example, Bucculatrix pyrivorella;
(c) moths of the family Carposinidae, for example, Carposina sasakii;
(d) moths of the family Crambidae, for example, Diaphania indica and Diaphania nitidalis of Diaphania spp.; for example, Ostrinia furnacalis, Ostrinia nubilalis, and Ostrinia scapulalis of Ostrinia spp.; and Chilo suppressalis, Cnaphalocrocis medinalis, Conogethes punctiferalis, Diatraea grandiosella, Glyphodes pyloalis, Hellula undalis, and Parapediasia teterrella;
(e) moths of the family Gelechiidae, for example, Helcystogramma triannulella, Pectinophora gossypiella, Phthorimaea operculella, and Sitotroga cerealella;
(f) moths of the family Geometridae, for example, Ascotis selenaria;
(g) moths of the family Gracillariidae, for example, Caloptilia theivora, Phyllocnistis citrella, and Phyllonorycter ringoniella;
(h) butterflies of the family Hesperiidae, for example, Parnara guttata;
(i) moths of the family Lasiocampidae, for example, Malacosoma neustria;
(j) moths of the family Lymantriidae, for example, Lymantria dispar and Lymantria monacha of Lymantria spp.; and Euproctis pseudoconspersa and Orgyia thyellina;
(k) moths of the family Lyonetiidae, for example, Lyonetia clerkella and Lyonetia prunifoliella malinella of Lyonetia spp.;
(1) moths of the family Noctuidae, for example, Spodoptera depravata, Spodoptera eridania, Spodoptera exigua, Spodoptera frugiperda, Spodoptera littoralis, and Spodoptera litura of Spodoptera spp.; for example, Autographa gamma and Autographa nigrisigna of Autographa spp.; for example, Agrotis ipsilon and Agrotis segetum of Agrotis spp.; for example, Helicoverpa armigera, Helicoverpa assulta, and Helicoverpa zea of Helicoverpa spp.; for example, Heliothis armigera and Heliothis virescens of Heliothis spp.; and Aedia leucomelas, Ctenoplusia agnata, Eudocima tyrannus, Mamestra brassicae, Mythimna separata, Naranga aenescens, Panolis japonica, Peridroma saucia, Pseudoplusia includens, and Trichoplusia ni;
(m) moths of the family Nolidae, for example, Earias insulana;
(n) butterflies of the family Pieridae, for example, Pieris brassicae and Pieris rapae crucivora of Pieris spp.;
(o) moths of the family Plutellidae, for example, Acrolepiopsis sapporensis and Acrolepiopsis suzukiella of Acrolepiopsis spp.; and Plutella xylostella;
(p) moths of the family Pyralidae, for example, Cadra cautella, Elasmopalpus lignosellus, Etiella zinckenella, and Galleria mellonella;
(q) moths of the family Sphingidae, for example, Manduca quinquemaculata and Manduca sexta of Manduca spp.;
(r) moths of the family Stathmopodidae, for example, Stathmopoda masinissa;
(s) moths of the family Tineidae, for example, Tinea translucens;
(t) moths of the family Tortricidae, for example, Adoxophyes honmai and Adoxophyes orana of Adoxophyes spp.; for example, Archips breviplicanus and Archips fuscocupreanus of Archips spp.; and Choristoneura fumiferana, Cydia pomonella, Eupoecilia ambiguella, Grapholitha molesta, Homona magnanima, Leguminivora glycinivorella, Lobesia botrana, Matsumuraeses phaseoli, Pandemis heparana, and Sparganothis pilleriana;
(u) moths of the family Yponomeutidae, for example, Argyresthia conjugella.
(2) Insect pests of the order Thysanoptera
(a) insect pests of the family Phlaeothripidae, for example, Ponticulothrips diospyrosi;
(b) insect pests of the family Thripidae, for example, Frankliniella intonsa and Frankliniella occidentalis of Frankliniella spp.; for example, Thrips palmi and Thrips tabaci of Thrips spp.; and Heliothrips haemorrhoidalis and Scirtothrips dorsalis.

### (3) Insect Pests of the Order Hemiptera

(A) the suborder Archaeorrhyncha
(a) insect pests of the family Delphacidae, for example, Laodelphax striatella, Nilaparvata lugens, Perkinsiella saccharicida, and Sogatella furcifera.
(B) the suborder Clypeorrhyncha
(a) insect pests of the family Cicadellidae, for example, Empoasca fabae, Empoasca nipponica, Empoasca onukii, and Empoasca sakaii of Empoasca spp.; and Arboridia apicalis, Balclutha saltuella, Epiacanthus stramineus, Macrosteles striifrons, and Nephotettix cinctinceps.
(C) the suborder Heteroptera
(a) insect pests of the family Alydidae, for example, Riptortus clavatus;
(b) insect pests of the family Coreidae, for example, Cletus punctiger and Leptocorisa chinensis;
(c) insect pests of the family Lygaeidae, for example, Blissus leucopterus, Cavelerius saccharivorus, and Togo hemipterus;
(d) insect pests of the family Miridae, for example, Halticus insularis, Lygus lineolaris, Psuedatomoscelis seriatus, Stenodema sibiricum, Stenotus rubrovittatus, and Trigonotylus caelestialium;
(e) insect pests of the family Pentatomidae, for example, Nezara antennata and Nezara viridula of Nezara spp.; for example, Eysarcoris aeneus, Eysarcoris lewisi, and Eysarcoris ventralis of Eysarcoris spp.; and Dolycoris baccarum, Eurydema rugosum, Glaucias subpunctatus, Halyomorpha halys, Piezodorus hybneri, Plautia crossota, and Scotinophora lurida;
(f) insect pests of the family Pyrrhocoridae, for example, Dysdercus cingulatus;
(g) insect pests of the family Rhopalidae, for example, Rhopalus msculatus;
(h) insect pests of the family Scutelleridae, for example, Eurygaster integriceps;
(i) insect pests of the family Tingidae, for example, Stephanitis nashi.
(D) the suborder Sternorrhyncha
(a) insect pests of the family Adelgidae, for example, Adelges laricis;
(b) insect pests of the family Aleyrodidae, for example, Bemisia argentifolii and Bemisia tabaci of Bemisia spp.; and Aleurocanthus spiniferus, Dialeurodes citri, and Trialeurodes vaporariorum;
(c) insect pests of the family Aphididae, for example, Aphis craccivora, Aphis fabae, Aphis forbesi, Aphis gossypii, Aphis pomi, Aphis sambuci, and Aphis spiraecola of Aphis spp.; for example, Rhopalosiphum maidis and Rhopalosiphum padi of Rhopalosiphum spp.; for example, Dysaphis plantaginea and Dysaphis radicola of Dysaphis spp.; for example, Macrosiphum avenae and Macrosiphum euphorbiae of Macrosiphum spp.; for example, Myzus cerasi, Myzus persicae, and Myzus varians of Myzus spp.; and Acyrthosiphon pisum, Aulacorthum solani, Brachycaudus helichrysi, Brevicoryne brassicae, Chaetosiphon fragaefolii, Hyalopterus pruni, Hyperomyzus lactucae, Lipaphis erysimi, Megoura viciae, Metopolophium dirhodum, Nasonovia ribis-nigri, Phorodon humuli, Schizaphis graminum, Sitobion avenae, and Toxoptera aurantii;
(d) insect pests of the family Coccidae, for example, Ceroplastes ceriferus and Ceroplastes rubens of Ceroplastes spp.;
(e) insect pests of the family Diaspididae, Pseudaulacaspis pentagona and Pseudaulacaspis prunicola of Pseudaulacaspis spp.; for example, Unaspis euonymi and Unaspis yanonensis of Unaspis spp.; and Aonidiella aurantii, Comstockaspis perniciosa, Fiorinia theae, and Pseudaonidia paeoniae;
(f) insect pests of the family Margarodidae, for example, Drosicha corpulenta and Icerya purchasi;
(g) insect pests of the family Phylloxeridae, for example, Viteus vitifolii;
(h) insect pests of the family Pseudococcidae, for example, Planococcus citri and Planococcus kuraunhiae of Planococcus spp.; and Phenacoccus solani and Pseudococcus comstocki;
(i) insect pests of the family Psyllidae, for example, Psylla mali and Psylla pyrisuga of Psylla spp.; and Diaphorina citri.
(4) Insect pests of the suborder Polyphaga
(a) insect pests of the family Anobiidae, for example, Lasioderma serricorne;
(b) insect pests of the family Attelabidae, for example, Byctiscus betulae and Rhynchites heros;
(c) insect pests of the family Bostrichidae, for example, Lyctus brunneus;
(d) insect pests of the family Brentidae, for example, Cylas formicarius;
(e) insect pests of the family Buprestidae, for example, Agrilus sinuatus;
(f) insect pests of the family Cerambycidae, for example, Anoplophora malasiaca, Monochamus alternatus, Psacothea hilaris, and Xylotrechus pyrrhoderus;
(g) insect pests of the family Chrysomelidae, for example, Bruchus pisorum and Bruchus rufimanus of Bruchus spp.; for example, Diabrotica barberi, Diabrotica undecimpunctata, and Diabrotica virgifera of Diabrotica spp.; for example, Phyllotreta nemorum and Phyllotreta striolata of Phyllotreta spp.; and Aulacophora femoralis, Callosobruchus chinensis, Cassida nebulosa, Chaetocnema concinna, Leptinotarsa decemlineata, Oulema oryzae, and Psylliodes angusticollis;
(h) insect pests of the family Coccinellidae, for example, Epilachna varivestis and Epilachna vigintioctopunctata of Epilachna spp.;
(i) insect pests of the family Curculionidae, for example, Anthonomus grandis and Anthonomus pomorum of Anthonomus spp.; for example, Sitophilus granarius and Sitophilus zeamais of Sitophilus spp.; and Echinocnemus squameus, Euscepes postfasciatus, Hylobius abietis, Hypera postica, Lissohoptrus oryzophilus, Otiorhynchus sulcatus, Sitona lineatus, and Sphenophorus venatus;
(j) insect pests of the family Elateridae, for example, Melanotus fortnumi and Melanotus tamsuyensis of Melanotus spp.;
(k) insect pests of the family Nitidulidae, for example, Epuraea domina;
(1) insect pests of the family Scarabaeidae, for example, Anomala cuprea and Anomala rufocuprea of Anomala spp.; and Cetonia aurata, Gametis jucunda, Heptophylla picea, Melolontha melolontha, and Popillia japonica;
(m) insect pests of the family Scolytidae, for example, Ips typographus;
(n) insect pests of the family Staphylinidae, for example, Paederus fuscipes;
(o) insect pests of the family Tenebrionidae, for example, Tenebrio molitor and Tribolium castaneum;
(p) insect pests of the family Trogossitidae, for example, Tenebroides mauritanicus.

### (5) Insect Pests of the Order Diptera

(A) the suborder Brachycera
(a) insect pests of the family Agromyzidae, for example, Liriomyza bryoniae, Liriomyza chinensis, Liriomyza sativae, and Liriomyza trifolii of Liriomyza spp.; and Chromatomyia horticola and Agromyza oryzae;
(b) insect pests of the family Anthomyiidae, for example, Delia platura and Delia radicum of Delia spp.; and Pegomya cunicularia;
(c) insect pests of the family Drosophilidae, for example, Drosophila melanogaster and Drosophila suzukii of Drosophila spp.;
(d) insect pests of the family Ephydridae, for example, Hydrellia griseola;
(e) insect pests of the family Psilidae, for example, Psila rosae;
(f) insect pests of the family Tephritidae, for example, Bactrocera cucurbitae and Bactrocera dorsalis of Bactrocera spp.; for example, Rhagoletis cerasi and Rhagoletis pomonella of Rhagoletis spp.; and Ceratitis capitata and Dacus oleae.
(B) the suborder Nematocera
(a) insect pests of the family Cecidomyiidae, for example, Asphondylia yushimai, Contarinia sorghicola, Mayetiola destructor, and Sitodiplosis mosellana.

### (6) Insect Pests of the Order Orthoptera

(a) insect pests of the family Acrididae, for example, Schistocerca americana and Schistocerca gregaria of Schistocerca spp.; and Chortoicetes terminifera, Dociostaurus maroccanus, Locusta migratoria, Locustana pardalina, Nomadacris septemfasciata, and Oxya yezoensis;
(b) insect pests of the family Gryllidae, for example, Acheta domestica and Teleogryllus emma;
(c) insect pests of the family Gryllotalpidae, for example, Gryllotalpa orientalis;
(d) insect pests of the family Tettigoniidae, for example, Tachycines asynamorus.

### (7) Acari

(A) Acaridida of the order Astigmata
(a) mites of the family Acaridae, for example, Rhizoglyphus echinopus and Rhizoglyphus robini of Rhizoglyphus spp.; for example, Tyrophagus neiswanderi, Tyrophagus perniciosus, Tyrophagus putrescentiae, and Tyrophagus similis of Tyrophagus spp.; and Acarus siro, Aleuroglyphus ovatus, and Mycetoglyphus fungivorus;
(B) Actinedida of the order Prostigmata
(a) mites of the family Tetranychidae, for example, Bryobia praetiosa and Bryobia rubrioculus of Bryobia spp.; for example, Eotetranychus asiaticus, Eotetranychus boreus, Eotetranychus celtis, Eotetranychus geniculatus, Eotetranychus kankitus, Eotetranychus pruni, Eotetranychus shii, Eotetranychus smithi, Eotetranychus suginamensis, and Eotetranychus uncatus of Eotetranychus spp.; for example, Oligonychus hondoensis, Oligonychus ilicis, Oligonychus karamatus, Oligonychus mangiferus, Oligonychus orthius, Oligonychus perseae, Oligonychus pustulosus, Oligonychus shinkajii, and Oligonychus ununguis of Oligonychus spp.; for example, Panonychus citri, Panonychus mori, and Panonychus ulmi of Panonychus spp.; for example, Tetranychus cinnabarinus, Tetranychus kanzawai, Tetranychus ludeni, Tetranychus quercivorus, Tetranychus phaselus, Tetranychus urticae, Tetranychus viennensis, and Tetranychus evansi of Tetranychus spp.; for example, Aponychus corpuzae and Aponychus firmianae of Aponychus spp.; for example, Sasanychus akitanus and Sasanychus pusillus of Sasanychus spp.; for example, Schizotetranychus celarius, Schizotetranychus longus, Schizotetranychus miscanthi, Schizotetranychus recki, and Schizotetranychus schizopus of Schizotetranychus spp.; and Tetranychina harti, Tuckerella pavoniformis, and Yezonychus sapporensis;
(b) mites of the family Tenuipalpidae, for example, Brevipalpus lewisi, Brevipalpus obovatus, Brevipalpus phoenicis, Brevipalpus russulus, and Brevipalpus californicus of Brevipalpus spp.; for example, Tenuipalpus pacificus and Tenuipalpus zhizhilashviliae of Tenuipalpus spp.; and Dolichotetranychus floridanus;
(c) mites of the family Eriophyidae, for example, Aceria diospyri, Aceria ficus, Aceria japonica, Aceria kuko, Aceria paradianthi, Aceria tiyingi, Aceria tulipae, and Aceria zoysiea of Aceria spp.; for example, Eriophyes chibaensis and Eriophyes emarginatae of Eriophyes spp.; for example, Aculops lycopersici and Aculops pelekassi of Aculops spp.; for example, Aculus fockeui and Aculus schlechtendali of Aculus spp.; and Acaphylla theavagrans, Calacarus carinatus, Colomerus vitis, Calepitrimerus vitis, Epitrimerus pyri, Paraphytoptus kikus, Paracalacarus podocarpi, and Phyllocotruta citri;
(d) mites of the family Tarsonemidae, for example, Tarsonemus bilobatus and Tarsonemus waitei of Tarsonemus spp.; and Phytonemus pallidus and Polyphagotarsonemus latus;
(e) mites of the family Penthaleidae, for example, Penthaleus erythrocephalus and Penthaleus major of Penthaleus spp.

The pest control agent of the present invention may be used as a mixture or in combination with another active ingredient such as a fungicide, an insecticide or miticide, a nematicide, or a pesticide for soil insect pests; a plant regulating agent, a synergist, a fertilizer, a soil improvement agent, animal feed, or the like.

The combination of the heteroaryl azole compound of the present invention with another active ingredient may be expected to have synergistic effects on insecticidal, miticidal, or nematicidal activity. The synergistic effects may be confirmed by the known method with the equation of Colby (Colby. S. R.; Calculating Synergistic and Antagonistic Responses of Herbicide Combinations; Weeds 15, p. 20-22, 1967) according to a standard method.

Specific examples of the insecticide or miticide, the nematicide, the pesticide for soil insect pests, the anthelmintic agent, and the like that may be used as a mixture or in combination with the pest control agent of the present invention will be shown below.

(1) Acetylcholinesterase inhibitors:
(a) carbamate-based: alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, oxamyl, pirimicarb, propoxur, thiodicarb, thiofanox, triazamate, trimethacarb, XMC, xylylcarb, fenothiocarb, MIPC, MPMC, MTMC, aldoxycarb, allyxycarb, aminocarb, bufencarb, chloethocarb, metam sodium, and promecarb;
(b) organophosphorus-based: acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isocarbophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion, bromophos-ethyl, BRP, carbophenothion, cyanofenphos, CYAP, demeton-S-methyl sulfone, dialifos, dichlofenthion, dioxabenzofos, etrimfos, fensulfothion, flupyrazofos, fonofos, formothion, fosmethilan, isazofos, iodofenphos, methacrifos, pirimiphos-ethyl, phosphocarb, propaphos, prothoate, and sulprofos.
(2) GABAergic chloride ion channel antagonists: acetoprole, chlordene, endosulfan, ethiprole, fipronil, pyrafluprole, pyriprole, camphechlor, heptachlor, and dienochlor.
(3) Sodium channel modulators: acrinathrin, d-cis-trans allethrin, d-trans allethrin, bifenthrin, bioallethrin, bioallethrin S-cyclopentyl isomers, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R)-isomers], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans isomers], prallethrin, pyrethrum, resmethrin, silafluofen, tefluthrin, tetramethrin [(1R)-isomers], tralomethrin, transfluthrin, allethrin, pyrethrin, pyrethrin I, pyrethrin II, profluthrin, dimefluthrin, bioethanomethrin, biopermethrin, transpermethrin, fenfluthrin, fenpirithrin, flubrocythrinate, flufenprox, metofluthrin, protrifenbute, pyresmethrin, and terallethrin.
(4) Nicotinic acetylcholine receptor agonists: acetamiprid, clothianidin, dinotefuran, imidacloprid, nitenpyram, nithiazine, thiacloprid, thiamethoxam, sulfoxaflor, nicotine, flupyradifurone, and flupyrimin.
(5) Nicotinic acetylcholine receptor allosteric modulators: spinetoram and spinosad.
(6) Chloride channel activators: abamectin, emamectin benzoate, lepimectin, milbemectin, ivermectin, selamectin, doramectin, eprinomectin, moxidectin, milbemycin, milbemycin oxime, and nemadectin.
(7) Juvenile hormone-like substances: hydroprene, kinoprene, methoprene, fenoxycarb, pyriproxyfen, diofenolan, epofenonane, and triprene.
(8) Other nonspecific inhibitors: methyl bromide, chloropicrin, sulfuryl fluoride, borax, and tartar emetic.
(9) Homoptera selective feeding inhibitors: flonicamid, pymetrozine, and pyrifluquinazon.
(10) Mite growth inhibitors: clofentezine, diflovidazin, hexythiazox, and etoxazole.
(11) Insect midgut inner membrane disrupting agents derived from microorganisms: Bacillus thuringiensis subsp. Isuraerenshi, Bacillus sphaericus, Bacillus thuringiensis subsp. Aizawai, Bacillus thuringiensis subsp. Kurstaki, Bacillus thuringiensis subsp. Tenebrionis, and Bt crop proteins: Cry1Ab, Cry1Ac, Cry1Fa, Cry1A.105, Cry2Ab, Vip3A, mCry3A, Cry3Ab, Cry3Bb, and Cry34Ab1/Cry35Ab1.
(12) Mitochondrial ATP biosynthetic enzyme inhibitors: diafenthiuron, azocyclotin, cyhexatin, fenbutatin oxide, propargite, and tetradifon.
(13) Oxidative phosphorylation uncouplers: chlorfenapyr, sulfluramid, DNOC, binapacryl, dinobuton, and dinocap.
(14) Nicotinic acetylcholine receptor channel blockers: bensultap, cartap hydrochloride, nereistoxin, thiosultap sodium, and thiocyclam.
(15) Chitin synthesis inhibitors: bistrifluron, chlorfluazuron, diflubenzuron, flucycloxuron, flufenoxuron, hexaflumuron, lufenuron, novaluron, noviflumuron, teflubenzuron, triflumuron, buprofezin, and fluazuron.
(16) Diptera molting disrupting agents: cyromazine.
(17) Molting hormone receptor agonists: chromafenozide, halofenozide, methoxyfenozide, and tebufenozide.
(18) Octopamine receptor agonists: amitraz, demiditraz, and chlordimeform.
(19) Mitochondrial electron transport system complex III inhibitors: acequinocyl, fluacrypyrim, and hydramethylnon.
(20) Mitochondrial electron transport system complex I inhibitors: fenazaquin, fenpyroximate, pyrimidifen, pyridaben, tebufenpyrad, tolfenpyrad, and rotenone.
(21) Voltage-gated sodium channel blockers: indoxacarb and metaflumizone.
(22) Acetyl CoA carboxylase inhibitors: spirodiclofen, spiromesifen, and spirotetramat.
(23) Mitochondrial electron transport system complex IV inhibitors: aluminum phosphide, calcium phosphide, phosphine, zinc phosphide, and cyanide.
(24) Mitochondrial electron transport system complex II inhibitors: cyenopyrafen, cyflumetofen, and pyflubumide.
(25) Ryanodine receptor modulators: chlorantraniliprole, cyantraniliprole, flubendiamide, cyclaniliprole, and tetraniliprole.
(26) Mixed function oxidase inhibitor compounds: piperonyl butoxide.
(27) Latrophilin receptor agonists: depsipeptide, cyclic depsipeptide, 24-membered cyclic depsipeptide, and emodepside.
(28) Other agents (based on an unknown mechanism of action): azadirachtin, benzoximate, bifenazate, bromopropylate, chinomethionate, cryolite, dicofol, pyridalyl, benclothiaz, sulfur, amidoflumet, 1,3-dichloropropene, DCIP, phenisobromolate, benzomate, metaldehyde, chlorobenzilate, clothiazoben, dicyclanil, fenoxacrim, fentrifanil, flubenzimin, fluphenazine, gossyplure, japonilure, metoxadiazone, petroleum, potassium oleate, tetrasul, triarathene, afidopyropen, flometoquin, flufiprole, fluensulfone, meperfluthrin, tetramethylfluthrin, tralopyril, dimefluthrin, methylneodecanamide, fluralaner, afoxolaner, fluxametamide, 5-[5-(3,5-dichlorophenyl)-5-trifluoromethyl-4,5-dihydroisoxazol-3-yl]-2-(1H-1,2,4-triazol-1-yl)benzonitrile (CAS: 943137-49-3), broflanilide, and other m-diamides.
(29) Anthelmintic agents:
(a) benzimidazole-based: fenbendazole, albendazole, triclabendazole, oxibendazole, mebendazole, oxfendazole, parbendazole, flubendazole, febantel, netobimin, thiophanate, thiabendazole, and cambendazole;
(b) salicylanilide-based: closantel, oxyclozanide, rafoxanide, and niclosamide;
(c) substituted phenol-based: nitroxinil and nitroscanate;
(d) pyrimidine-based: pyrantel and morantel;
(e) imidazothiazole-based: levamisole and tetramisole;
(f) tetrahydropyrimidine-based: praziquantel and epsiprantel;
(g) other anthelmintic agents: cyclodiene, ryania, clorsulon, metronidazole, demiditraz, piperazine, diethylcarbamazine, dichlorophene, monepantel, tribendimidine, amidantel, thiacetarsamide, melarsomine, and arsenamide.

Specific examples of the fungicide that may be used as a mixture or in combination with the pest control agent of the present invention will be shown below.

(1) Nucleic acid biosynthesis inhibitors:
(a) RNA polymerase I inhibitors: benalaxyl, benalaxyl-M, furalaxyl, metalaxyl, metalaxyl-M, oxadixyl, clozylacon, and ofurace;
(b) adenosine deaminase inhibitors: bupirimate, dimethirimol, and ethirimol;
(c) DNA/RNA synthesis inhibitors: hymexazol and octhilinone;
(d) DNA topoisomerase II inhibitors: oxolinic acid.
(2) Mitotic inhibitors and cell division inhibitors:
(a) β-tubulin polymerization inhibitors: benomyl, carbendazim, chlorfenazole, fuberidazole, thiabendazole, thiophanate, thiophanate-methyl, diethofencarb, zoxamide, and ethaboxam;
(b) cell division inhibitors: pencycuron;
(c) spectrin-like protein delocalization inhibitors: fluopicolide.
(3) Respiration inhibitors:
(a) complex I NADH oxidation-reduction enzyme inhibitors: diflumetorim and tolfenpyrad;
(b) complex II succinate dehydrogenase inhibitors: benodanil, flutolanil, mepronil, isofetamid, fluopyram, fenfuram, furmecyclox, carboxin, oxycarboxin, thifluzamide, benzovindiflupyr, bixafen, fluxapyroxad, furametpyr, isopyrazam, penflufen, penthiopyrad, sedaxane, and boscalid;
(c) complex III ubiquinol oxidase Qo inhibitors: azoxystrobin, coumoxystrobin, coumethoxystrobin, enoxastrobin, flufenoxystrobin, picoxystrobin, pyraoxystrobin, pyraclostrobin, pyrametostrobin, triclopyricarb, kresoxim-methyl, trifloxystrobin, dimoxystrobin, fenaminstrobin, metominostrobin, orysastrobin, famoxadone, fluoxastrobin, fenamidone, and pyribencarb;
(d) complex III ubiquinol reductase Qi inhibitors: cyazofamid and amisulbrom;
(e) oxidative phosphorylation uncoupling agents: binapacryl, meptyldinocap, dinocap, fluazinam, and ferimzone;
(f) oxidative phosphorylation inhibitors (ATP synthase inhibitors): fentin acetate, fentin chloride, and fentin hydroxide;
(g) ATP production inhibitors: silthiofam;
(h) complex III: Qx (unknown) inhibitor of cytochrome bcl (ubiquinone reductase): ametoctradin.
(4) Amino acid and protein synthesis inhibitors
(a) methionine biosynthesis inhibitors: andoprim, cyprodinil, mepanipyrim, and pyrimethanil;
(b) protein synthesis inhibitors: blasticidin-S, kasugamycin, kasugamycin hydrochloride, streptomycin, and oxytetracycline.
(5) Signal transduction inhibitors:
(a) signal transduction inhibitors: quinoxyfen and proquinazid;
(b) MAP/histidine kinase inhibitors in osmotic signal transduction: fenpiclonil, fludioxonil, chlozolinate, iprodione, procymidone, and vinclozolin.
(6) Lipid and cell membrane synthesis inhibitors:
(a) phospholipid biosynthesis, methyltransferase inhibitors: edifenphos, iprobenfos, pyrazophos, and isoprothiolane;
(b) lipid peroxidation agents: biphenyl, chloroneb, dicloran, quintozene, tecnazene, tolclofos-methyl, and etridiazole;
(c) agents that act on cell membranes: iodocarb, propamocarb, propamocarb hydrochloride, propamocarb fosetylate, and prothiocarb;
(d) microorganisms that disrupt cell membranes of pathogens: Bacillus subtilis bacteria, Bacillus subtilis QST713 strain, Bacillus subtilis FZB24 strain, Bacillus subtilis MBI600 strain, and Bacillus subtilis D747 strain;
(e) agents that disrupt cell membranes: extracts of Melaleuca alternifolia (tea tree).
(7) Cell membrane sterol biosynthesis inhibitors:
(a) C14-demethylation inhibitors in sterol biosynthesis: triforine, pyrifenox, pyrisoxazole, fenarimol, flurprimidol, nuarimol, imazalil, imazalil sulfate, oxpoconazole, pefurazoate, prochloraz, triflumizole, viniconazole, azaconazole, bitertanol, bromuconazole, cyproconazole, diclobutrazol, difenoconazole, diniconazole, diniconazole-M, epoxiconazole, etaconazole, fenbuconazole, fluquinconazole, flusilazole, flutriafol, fluconazole, fluconazole-cis, hexaconazole, imibenconazole, ipconazole, metconazole, myclobutanil, penconazole, propiconazole, quinconazole, simeconazole, tebuconazole, tetraconazole, triadimefon, triadimenol, triticonazole, prothioconazole, and voriconazole;
(b) 014 reductase and sterol 08-A7-isomerase inhibitors in sterol biosynthesis: aldimorph, dodemorph, dodemorph acetate, fenpropimorph, tridemorph, fenpropidin, piperalin, and spiroxamine;
(c) 3-keto reductase inhibitors in C4-demethylation in the sterol biosynthesis system: fenhexamid and fenpyrazamine;
(d) squalene epoxidase inhibitors in the sterol biosynthesis system: pyributicarb, naftifine, and terbinafine.
(8) Cell wall synthesis inhibitors
(a) trehalase inhibitors: validamycin;
(b) chitin synthase inhibitors: polyoxin and polyoxorim;
(c) cellulose synthase inhibitors: dimethomorph, flumorph, pyrimorph, benthiavalicarb, iprovalicarb, tolprocarb, valifenalate, and mandipropamid.
(9) Melanin biosynthesis inhibitors
(a) melanin biosynthesis reductase inhibitors: fthalide, pyroquilon, and tricyclazole;
(b) melanin biosynthesis anhydrase inhibitors: carpropamid, diclocymet, and fenoxanil;
(10) Host plant resistance inducers:
(a) agents that act on salicylic acid synthesis pathway: acibenzolar-S-methyl;
(b) others: probenazole, tiadinil, isotianil, laminarin, and giant knotweed extracts.
(11) Agents with unknown mode of action: cymoxanil, fosetyl aluminum, phosphoric acid (phosphate), tecloftalam, triazoxide, flusulfamide, diclomezine, methasulfocarb, cyflufenamid, metrafenone, pyriofenone, dodine, dodine free base, and flutianil.
(12) Agents having multiple active sites: copper (copper salt), Bordeaux mixture, copper hydroxide, copper naphthalate, copper oxide, copper oxychloride, copper sulfate, sulfur, sulfur products, calcium polysulfide, ferbam, mancozeb, maneb, mancopper, metiram, polycarbamate, propineb, thiram, zineb, ziram, captan, captafol, folpet, chlorothalonil, dichlofluanid, tolylfluanid, guazatine, iminoctadine triacetate, iminoctadine trialbesilate, anilazine, dithianon, chinomethionate, and fluoroimide.
(13) Other agents: DBEDC, fluoro folpet, guazatine acetate, bis(8-quinolinolato)copper (II), propamidine, chloropicrin, cyprofuram, agrobacterium, bethoxazin, diphenylamine, methyl isothiocyanate (MITC), mildiomycin, capsaicin, cufraneb, cyprosulfamide, dazomet, debacarb, dichlorophene, difenzoquat, difenzoquat methyl sulfonate, flumetover, fosetyl calcium, fosetyl sodium, irumamycin, natamycin, nitrothal-isopropyl, oxamocarb, propanosine sodium, pyrrolnitrin, tebufloquin, tolnifanide, zarilamid, algophase, amicarthiazol, oxathiapiprolin, metiram zinc, benthiazole, trichlamide, uniconazole, oxyfenthiin, and picarbutrazox.

Specific examples of the plant regulating agent that may be used as a mixture or in combination with the pest control agent of the present invention will be shown below.

Abscisic acid, kinetin, benzylaminopurine, 1,3-diphenylurea, forchlorfenuron, thidiazuron, chlorfenuron, dihydrozeatin, gibberellin A, gibberellin A4, gibberellin A7, gibberellin A3, 1-methylcyclopropane, N-acetylaminoethoxyvinylglycine (also called aviglycine), aminooxyacetic acid, silver nitrate, cobalt chloride, IAA, 4-CPA, cloprop, 2,4-D, MCPB, indole-3-butyric acid, dichlorprop, phenothiol, 1-naphthyl acetamide, ethychlozate, cloxyfonac, maleic acid hydrazide, 2,3,5-triiodobenzoic acid, salicylic acid, methyl salicylate, (−)-jasmonic acid, methyl jasmonate, (+)-strigol, (+)-deoxystrigol, (+)-orobanchol, (+)-sorgolactone, 4-oxo-4-(2-phenylethyl)aminobutyric acid, ethephon, chlormequat, mepiquat chloride, benzyl adenine, and 5-aminolevulinic acid.

### [Ectoparasite Control Agent]

The ectoparasite control agent of the present invention contains at least one active ingredient selected from the heteroaryl azole compounds of the present invention. The amount of the heteroaryl azole compound contained in the ectoparasite control agent of the present invention is not particularly limited as long as its ectoparasite control effect is exhibited.

As the host animal to be treated with the ectoparasite control agent of the present invention, a warm-blooded animal such as a human and a livestock mammal (e.g., a cow, a horse, a pig, sheep, and a goat), a laboratory animal (e.g., a mouse, a rat, and a sand rat), a pet animal (e.g., a hamster, a guinea pig, a dog, a cat, a horse, a squirrel, a rabbit, and a ferret), wild and zoo mammals (e.g., a monkey, a fox, a deer, and a buffalo), a fowl (e.g., a turkey, a duck, a chicken, a quail, and a goose), and a pet bird (e.g., a pigeon, a parrot, a myna bird, a Java sparrow, a parakeet, a Bengalese finch, and a canary bird); and fishes such as salmon, trout, and carp may be exemplified. In addition, a bee, a stag beetle and a beetle may be exemplified.

The ectoparasite control agent of the present invention may be applied by a known veterinary method (local, oral, parenteral or subcutaneous administration). As the method therefor, a method of orally administering tablets, capsules, feed or the like containing the ectoparasite control agent to animals; a method of administering the ectoparasite control agent with dipping liquids, suppositories, injection (intramuscular, subcutaneous, intravenous, or intraperitoneal injection, etc.) or the like to animals; a method of locally administering oily or aqueous liquid formulations by spraying, pour-on, spot-on or the like; and a method of locally administering the ectoparasite control agent to animals by attaching to animals a material such as a collar or an ear tag produced by suitably shaping the resin into which the ectoparasite control agent is kneaded may be exemplified.

Ectoparasites parasitize the inside or the body surface of host animals, particularly, warm-blooded animals. Specifically, the ectoparasites parasitize the backs, armpits, lower abdomens, inner thighs, or the like of host animals and live by obtaining nutrients such as blood or dandruff from the animals. As the ectoparasite, mites, lice, fleas, a mosquito, a stable fly, a flesh fly or the like may be exemplified. Specific examples of the ectoparasite controllable with the ectoparasite control agent of the present invention will be shown below.

### (1) Acari

mites of the family Dermanyssidae, mites of the family Macronyssidae, mites of the family Laelapidae, mites of the family Varroidae, mites of the family Argasidae, mites of the family Ixodidae, mites of the family Psoroptidae, mites of the family Sarcoptidae, mites of the family Knemidokoptidae, mites of the family Demodixidae, mites of the family Trombiculidae, and insect parasitic mites such as mites of the family Canestriniidae.
(2) The order Phthiraptera
lice of the family Haematopinidae, lice of the family Linognathidae, bird lice of the family Menoponidae, bird lice of the family Philopteridae, and bird lice of the family Trichodectidae.
(3) The order Siphonaptera fleas of the family Pulicidae, for example, Ctenocephalides canis and Ctenocephalides felis of Ctenocephalides spp.;
fleas of the family Tungidae, fleas of the family Ceratophyllidae, and fleas of the family Leptopsyllidae.
(4) The order Hemiptera
(5) Insect pests of the order Diptera mosquitos of the family Culicidae, black flies of the family Simuliidae, biting midges of the family Ceratopogonidae, horseflies of the family Tabanidae, flies of the family Muscidae, tsetse flies of the family Glossinidae, flesh flies of the family Sarcophagidae, flies of the family Hippoboscidae, flies of the family Calliphoridae, and flies of the family Oestridae.

### [Endoparasite Control Agent or Expellant]

The endoparasite control agent or expellant of the present invention contains at least one active ingredient selected from the heteroaryl azole compounds of the present invention. The amount of the heteroaryl azole compound contained in the endoparasite control agent or expellant of the present invention is not particularly limited as long as its endoparasite control effect is exhibited.

The parasite targeted by the endoparasite control agent or expellant of the present invention parasitizes the inside of host animals, particularly, warm-blooded animals or fishes (endoparasite). As the host animal for which the endoparasite control agent or expellant of the present invention is effective, a warm-blooded animal such as a human and a livestock mammal (e.g., a cow, a horse, a pig, sheep, and a goat), a laboratory animal (e.g., a mouse, a rat, and a sand rat), a pet animal (e.g., a hamster, a guinea pig, a dog, a cat, a horse, a squirrel, a rabbit, and a ferret), wild and zoo mammals (e.g., a monkey, a fox, a deer, and a buffalo), a fowl (e.g., a turkey, a duck, a chicken, a quail, and a goose), and a pet bird (e.g., a pigeon, a parrot, a myna bird, a Java sparrow, a parakeet, a Bengalese finch, and a canary bird); and fishes such as salmon, trout, and carp may be exemplified. Parasitic diseases mediated by parasites may be prevented or treated by controlling and expelling the parasites.

As the parasite to be controlled or expelled, the following may be exemplified.

(1) Nematodes of the order Dioctophymatida
(a) kidney worms of the family Dioctophymatidae, for example, Dioctophyma renale of Dioctophyma spp.;
(b) kidney worms of the family Soboliphymatidae, for example, Soboliphyme abei and Soboliphyme baturini of Soboliphyme spp.
(2) Nematodes of the order Trichocephalida
(a) trichinae of the family Trichinellidae, for example, Trichinella spiralis of Trichinella spp.;
(b) whipworms of the family Trichuridae, for example, Capillaria annulata, Capillaria contorta, Capillaria hepatica, Capillaria perforans, Capillaria plica, and Capillaria suis of Capillaria spp.; and Trichuris vulpis, Trichuris discolor, Trichuris ovis, Trichuris skrjabini, and Trichuris suis of Trichuris spp.
(3) Nematodes of the order Rhabditida
threadworms of the family Strongyloididae, for example, Strongyloides papillosus, Strongyloides planiceps, Strongyloides ransomi, Strongyloides suis, Strongyloides stercoralis, Strongyloides tumefaciens, and Strongyloides ratti of Strongyloides spp.
(4) Nematodes of the order Strongylida
hookworms of the family Ancylostomatidae, for example, Ancylostoma braziliense, Ancylostoma caninum, Ancylostoma duodenale, and Ancylostoma tubaeforme of Ancylostoma spp.; Uncinaria stenocephala of Uncinaria spp.; and Bunostomum phlebotomum and Bunostomum trigonocephalum of Bunostomum spp.
(5) Nematodes of the order Strongylida
(a) nematodes of the family Angiostrongylidae, for example, Aelurostrongylus abstrusus of Aelurostrongylus spp.; and Angiostrongylus vasorum and Angiostrongylus cantonesis of Angiostrongylus spp.;
(b) nematodes of the family Crenosomatidae, for example, Crenosoma aerophila and Crenosoma vulpis of Crenosoma spp.;
(c) nematodes of the family Filaroididae, for example, Filaroides hirthi and Filaroides osleri of Filaroides spp.;
(d) lungworms of the family Metastrongylidae, for example, Metastrongylus apri, Metastrongylus asymmetricus, Metastrongylus pudendotectus, and Metastrongylus salmi of Metastrongylus spp.;
(e) gapeworms of the family Syngamidae, for example, Cyathostoma bronchialis of Cyathostoma spp.; and Syngamus skrjabinomorpha and Syngamus trachea of Syngamus spp.
(6) Nematodes of the order Strongylida
(a) nematodes of the family Molineidae, for example, Nematodirus filicollis and Nematodirus spathiger of Nematodirus spp.;
(b) nematodes of the family Dictyocaulidae, for example, Dictyocaulus filaria and Dictyocaulus viviparus of Dictyocaulus spp.;
(c) nematodes of the family Haemonchidae, for example, Haemonchus contortus of Haemonchus spp.; and Mecistocirrus digitatus of Mecistocirrus spp.;
(d) nematodes of the family Haemonchidae, for example, Ostertagia ostertagi of Ostertagia spp.;
(e) nematodes of the family Heligmonellidae, for example, Nippostrongylus braziliensis of Nippostrongylus spp.;
(f) nematodes of the family Trichostrongylidae, for example, Trichostrongylus axei, Trichostrongylus colubriformis, and Trichostrongylus tenuis of Trichostrongylus spp.; Hyostrongylus rubidus of Hyostrongylus spp.; and Obeliscoides cuniculi of Obeliscoides spp.
(7) Nematodes of the order Strongylida
(a) nematodes of the family Chabertiidae, for example, Chabertia ovina of Chabertia spp.; and Oesophagostomum brevicaudatum, Oesophagostomum columbianum, Oesophagostomum dentatum, Oesophagostomum georgianum, Oesophagostomum maplestonei, Oesophagostomum quadrispinulatum, Oesophagostomum radiatum, Oesophagostomum venulosum, and Oesophagostomum watanabei of Oesophagostomum spp.;
(b) nematodes of the family Stephanuridae, for example, Stephanurus dentatus of Stephanurus spp.;
(c) nematodes of the family Strongylidae, for example, Strongylus asini, Strongylus edentatus, Strongylus equinus, and Strongylus vulgaris of Strongylus spp.
(8) Nematodes of the order Oxyurida
nematodes of the family Oxyuridae, for example, Enterobius anthropopitheci and Enterobius vermicularis of Enterobius spp.; Oxyuris equi of Oxyuris spp.; and Passalurus ambiguous of Passalurus spp.
(9) Nematodes of the order Ascaridida
(a) nematodes of the family Ascaridiidae, for example, Ascaridia galli of Ascaridia spp.;
(b) nematodes of the family Heterakidae, for example, Heterakis beramporia, Heterakis brevispiculum, Heterakis gallinarum, Heterakis pusilla, and Heterakis putaustralis of Heterakis spp.;
(c) nematodes of the family Anisakidae, for example, Anisakis simplex of Anisakis spp.;
(d) nematodes of the family Ascarididae, for example, Ascaris lumbricoides and Ascaris suum of Ascaris spp.; and Parascaris equorum of Parascaris spp.;
(e) nematodes of the family Toxocaridae, for example, Toxocara canis, Toxocara leonina, Toxocara suum, Toxocara vitulorum, and Toxocara cati of Toxocara spp.
(10) Nematodes of the order Spirurida
(a) nematodes of the family Onchocercidae, for example, Brugia malayi, Brugia pahangi, and Brugia patei of Brugia spp.; Dipetalonema reconditum of Dipetalonema spp.; Dirofilaria immitis of Dirofilaria spp.; Filaria oculi of Filaria spp.; and Onchocerca cervicalis, Onchocerca gibsoni, and Onchocerca gutturosa of Onchocerca spp.;
(b) nematodes of the family Setariidae, for example, Setaria digitata, Setaria equina, Setaria labiatopapillosa, and Setaria marshalli of Setaria spp.; and Wuchereria bancrofti of Wuchereria spp.;
(c) nematodes of the family Filariidae, for example, Parafilaria multipapillosa of Parafilaria spp.; and Stephanofilaria assamensis, Stephanofilaria dedoesi, Stephanofilaria kaeli, Stephanofilaria okinawaensis, and Stephanofilaria stilesi of Stephanofilaria spp.
(11) Nematodes of the order Spirurida
(a) nematodes of the family Gnathostomatidae, for example, Gnathostoma doloresi and Gnathostoma spinigerum of Gnathostoma spp.;
(b) nematodes of the family Habronematidae, for example, Habronema majus, Habronema microstoma, and Habronema muscae of Habronema spp.; and Draschia megastoma of Draschia spp.;
(c) nematodes of the family Physalopteridae, for example, Physaloptera canis, Physaloptera cesticillata, Physaloptera erdocyona, Physaloptera felidis, Physaloptera gemina, Physaloptera papilloradiata, Physaloptera praeputialis, Physaloptera pseudopraerutialis, Physaloptera rara, Physaloptera sibirica, and Physaloptera vulpineus of Physaloptera spp.;
(d) nematodes of the family Gongylonematidae, for example, Gongylonema pulchrum of Gongylonema spp.;
(e) nematodes of the family Spirocercidae, for example, Ascarops strongylina of Ascarops spp.;
(f) nematodes of the family Thelaziidae, for example, Thelazia callipaeda, Thelazia gulosa, Thelazia lacrymalis, Thelazia rhodesi, and Thelazia skrjabini of Thelazia spp.

### [Control Agent for Other Pests]

The heteroaryl azole compound of the present invention is additionally excellent in control effect on insect pests that have a stinger or venom and harm humans and animals, insect pests that mediate various pathogens or disease-causing microbes, or insect pests that cause discomfort to humans (toxic pests, hygienic pests, and obnoxious pests, etc.).

Specific examples thereof will be shown below.

(1) Insect pests of the order Hymenoptera bees of the family Argidae, bees of the family Cynipidae, bees of the family Diprionidae, ants of the family Formicidae, bees of the family Mutillidae, and bees of the family Vespidae.

### (2) Other Insect Pests

Blattodea, termites, Araneae, centipedes, millipedes, crustacea, and Cimex lectularius.

### [Seed Treatment Agent or Vegetative Propagation Organ Treatment Agent]

The seed treatment agent or vegetative propagation organ treatment agent of the present invention contains at least one active ingredient selected from the heteroaryl azole compounds of the present invention. The amount of the heteroaryl azole compound contained in the seed treatment agent or vegetative propagation organ treatment agent of the present invention is not particularly limited as long as its control effect is exhibited.

The vegetative propagation organ means a plant root, stem, leaf, or the like having the ability to grow when the site is separated from the body and placed in soil. As the vegetative propagation organ, for example, a tuberous root, a creeping root, a bulb, a corm or solid bulb, a tuber, a rhizome, a stolon, a rhizophore, a cane cutting, a propagule and a vine cutting are exemplified. The stolon is also called runner. The propagule is also called bulblet and is classified into a broad bud and a bulbil. The vine cutting means a shoot (generic name for leaves and stems) of sweet potato, Japanese yam, or the like. [symbol] The bulb, the corm or solid bulb, the tuber, the rhizome, the cane cutting, the rhizophore and the tuberous root are also collectively called flower bulb. The cultivation of tubers and roots is started by planting tubers in soil. The tubers used are generally called seed tubers.

The seed treatment agent or vegetative propagation organ treatment agent of the present invention refers to a formulation such as a wettable powder, wettable granules, a flowable concentrate, or a dust produced by mixing at least one active ingredient selected from the heteroaryl azole compounds of the present invention with an appropriate solid carrier or liquid carrier, and, if necessary, adding a surfactant or other pharmaceutical aids to the mixture. This composition is usually used as a mixture with a binder. Also, the composition may contain a binder. As the composition containing a binder, a flowable concentrate (FS) for seed treatment is exemplified.

An adhesive substance that does not phytotoxically affect plant seeds or vegetative propagation organs is used as the binder. Specifically, at least one component selected from the group consisting of polyvinyl acetate, polyvinyl alcohol, cellulose including ethylcellulose, methylcellulose, hydroxymethylcellulose, hydroxypropylcellulose, and carboxymethylcellulose, polyvinylpyrrolidone, starch, modified starch, dextrin, maltodextrin, polysaccharides including alginate and chitosan, proteins including gelatin and casein, gum arabic, shellac, calcium lignosulfonate, and a methacrylamide monomer may be used.

Pests may be efficiently controlled by treating seed tubers with at least one compound selected from the heteroaryl azole compounds of the present invention. As the method for treating a seed tuber with the heteroaryl azole compound, dipping treatment, dust coating treatment, coating treatment or the like is exemplified. For the planting of seed tubers using a tractor, the seed tubers may be treated by spraying a chemical containing the heteroaryl azole compound onto the seed tubers on the tractor.

### [Granular Agrochemical Composition for Paddy Rice Seedling Nursery Box Treatment]

The granular agrochemical composition for paddy rice seedling nursery box treatment (hereinafter, referred to as the “granular agrochemical composition”) of the present invention contains at least one active ingredient selected from the heteroaryl azole compounds of the present invention. The amount of the heteroaryl azole compound contained in the granular agrochemical composition of the present invention is not particularly limited as long as its control effect is exhibited.

The granular agrochemical composition may be obtained by various methods, but is obtained, for example, by the extrusion granulation or the like of a powdery composition produced with the mixture of one or more active ingredients with, if necessary, a surfactant, a binder and an inorganic carrier, etc. with a wet granulation method. More specifically, a granular agrochemical composition is prepared by mixing uniformly the active ingredient adjusted to a predetermined particle size with a necessary surfactant, binder and inorganic carrier, adding an appropriate amount of water thereto, kneading the mixture, shaping them by extrusion through a screen having opened pores, and drying them. The size of the pores used in this operation is usually preferably 0.5 mm to 1.5 mm.

The particle size of the granular agrochemical composition thus obtained is not particularly limited, but is preferably 0.5 mm to 1.5 mm, and particularly preferably 0.7 mm to 1.5 mm, in terms of average particle size. The granules having such a particle size are obtained by granulation, drying and subsequent sifting.

The granular agrochemical composition is blended, if necessary, with a surfactant, a binder and an inorganic carrier. Among them, as the surfactant, for example, a nonionic surfactant such as polyethylene glycol higher fatty acid ester, polyoxyethylene alkyl ether, polyoxyethylene alkyl aryl ether, polyoxyethylene aryl phenyl ether, and sorbitan monoalkylate; a polycarboxylic acid-type polymer activator such as alkyl aryl sulfonate, dialkyl sulfonate, alkyl sulfuric acid ester salt, alkyl phosphoric acid ester salt, alkyl aryl sulfuric acid ester salt, alkyl aryl phosphoric acid ester salt, polyoxyethylene alkyl ether sulfuric acid ester salt, naphthalenesulfonate and a condensate thereof, ligninsulfonate, a copolymer of acrylic acid and itaconic acid or a copolymer of methacrylic acid and itaconic acid, a copolymer of maleic acid and styrene, a copolymer of maleic acid and diisobutylene and their alkali metal salts; polyoxyethylene aryl phenyl ether phosphoric acid ester salt; an anionic surfactant such as polyoxyethylene aryl phenyl ether sulfuric acid ester salt, or the like is exemplified. The amount of this surfactant added is usually 0.1 parts by weight to 5 parts by weight, though the amount is not particularly limited.

As the binder to be blended to the granular agrochemical composition, for example, carboxymethylcellulose metal salt, polyvinyl alcohol, pregelatinized starch, dextrin, xanthan gum, guar seed gum, sucrose, polyvinylpyrrolidone, polyacrylic acid metal salt or the like is exemplified. The amount of the binder added is usually 0.1 parts by weight to 5 parts by weight, though the amount is not particularly limited. The inorganic carrier to be blended to the granular agrochemical composition is not particularly limited. For example, clays, calcium carbonate, talc, diatomaceous earth, zeolite, attapulgite, gypsum, porcelain stone or the like is exemplified.

### [Soil Treatment Agent]

The soil treatment according to the present invention is, for example, a method of directly controlling pests by applying an active ingredient to the rhizospheres of plants to be protected from harm such as eating by the pest, or controlling pests by penetrating and transporting an active ingredient to the inside of plant bodies from their roots or the like. Specifically, for example, planting hole treatment (planting hole spraying and planting hole soil-incorporation), plant foot treatment (plant foot spraying, plant foot soil-incorporation, plant foot irrigation, and plant foot treatment at latter half of the seedling raising period), planting furrow treatment (planting furrow spraying and planting furrow soil-incorporation), planting row treatment (planting row spraying, planting row soil-incorporation, and planting row spraying at the growing period), planting row treatment at sowing (planting row spraying at sowing and planting row soil-incorporation at sowing), broadcast treatment (broadcast soil spraying and broadcast soil-incorporation), band dressing, submerged treatment (broadcast submerged application and frame submerged application), other soil spraying treatments (foliar granule spraying at the growing period, spraying under tree crowns or around main stems, soil surface spraying, soil surface incorporation, sowing hole spraying, surface spraying on the ribbing ground, and inter-plant spraying), other irrigation treatments (soil irrigation, irrigation at the seedling raising period, chemical injection treatment, irrigation on the plant foot, chemical drip irrigation, and chemigation), seedling nursery box treatment (seedling nursery box spraying, seedling nursery box irrigation, and seedling nursery box chemical flooding), seedling nursery tray treatment (seedling nursery tray spraying, seedling nursery tray irrigation, and flooded nursery tray spraying), nursery bed treatment (nursery bed spraying, nursery bed irrigation, flooded nursery bed spraying), seedling dipping, nursery soil-incorporation treatment (seedbed soil-incorporation and cover soil-incorporation), spraying before soil covering at sowing, spraying after soil covering at sowing, stem injection treatment, trunk injection treatment, trunk spraying treatment, and other treatments (plowing, surface soil-incorporation, soil incorporation into rain dropping lines, planting spot treatment, flower cluster granule spraying, and paste fertilizer mixing) are exemplified.

Water culture medium treatment is, for example, a method of protecting plants from harm ascribable to pests by applying an active ingredient to water culture media or the like and thereby penetrating and transporting an active ingredient to the inside of bodies of the plants to be protected from harm such as eating by the pests, from their roots or the like. Specifically, for example, water culture medium incorporation, water culture medium mixing or the like is exemplified.

The active ingredient may be applied to a plant by a method such as wrapping around the plant, tacking across the neighborhood of the plant, spreading over plant foot soil, or covering a cultivation region of the plant with a formulation allowed to contain the composition of the present invention by a method such as dipping, impregnation, coating, or kneading in a carrier such as a sheet-, strap-, tape- or net-shaped resin, paper, or cloth.

### [Bait Agent]

The bait agent of the present invention contains at least one active ingredient selected from the heteroaryl azole compounds of the present invention. The amount of the heteroaryl azole compound contained in the bait agent of the present invention is not particularly limited as long as its control effect is exhibited.

The bait agent of the present invention may contain, if necessary, an ingestibility improving component such as a sugar, a carbohydrate, or a milk constituent, a synergist, an aversive agent to prevent accidental ingestion, a preservative, a flavor, an attractant, or the like. The bait agent of the present invention may usually be prepared by mixing the heteroaryl azole compound with water and, if necessary, other components described above. In the preparation of the bait agent of the present invention, the heteroaryl azole compound may be the heteroaryl azole compound itself, or may be in the form of a preparation such as a dust, a wettable powder, a microcapsule, or a flowable concentrate.

As the pest that may be effectively controlled with the bait agent of the present invention, a cockroach such as Periplaneta americana, Blattella germanica, and Periplaneta fuliginosa, a click beetle such as Melanotus okinawensis, an ant such as Monomorium intrudens and Formica fusca, a deathwatch beetle such as Lasioderma serricorne and Stegobium paniceum, a flour beetle such as Tribolium castaneum and Tribolium confusum, a flat bark beetle such as Oryzaephilus surinamensis and Cryptolestes pusillus, a white ant such as Coptotermes formosanus and Reticulitermes speratus, a fly such as Musca domestica, Fannia canicularis, Phoridae, and Phlebotominae, and a mosquito such as Culex pipiens, Aedes albopictus, anopheles, and chironomids are exemplified.

The form of application of the bait agent of the present invention may be the bait agent of the present invention itself or may be, for example, in a state where a nonwoven fabric, sponge, absorbent cotton, or the like is impregnated with the bait agent. This aqueous bait agent or the nonwoven fabric, sponge, absorbent cotton, or the like impregnated with the bait agent is placed in a container, for example, a cup, a tray, or a bottle, and subjected to insect pest expelling. In this respect, a cover is put on the outside of the bait-containing container of the present invention to prepare an apparatus having some degree of space where insect pests are capable of residing in order to ingest the bait agent of the present invention. This generally improves ingestibility and is thus effective.

### [Plant Growth Promoter]

The plant growth promoter of the present invention contains at least one active ingredient selected from the heteroaryl azole compounds of the present invention. The amount of the heteroaryl azole compound contained in the plant growth promoter of the present invention is not particularly limited as long as its effect of promoting plant growth is exhibited.

The plant growth promoter of the present invention may be appropriately blended, if necessary, with other components, a carrier, or the like.

The heteroaryl azole compound of the present invention alone may be used as a plant growth promoter, but may usually be used as a formulation such as a wettable powder, a liquid formulation, an oil formulation, a dust, a granular formulation, or a suspension concentrate (flowable) by mixing the heteroaryl azole compound as an active ingredient with common adjuvants such as a solid carrier, a liquid carrier, a dispersant, a diluent, an emulsifier, a spreading agent and a thickener.

As the solid carrier or the liquid carrier, for example, talc, clay, bentonite, kaolin, diatomaceous earth, montmorillonite, mica, vermiculite, gypsum, calcium carbonate, white carbon, wood flour, starch, alumina, silicate, glycopolymer, waxes, water, alcohols (methyl alcohol, ethyl alcohol, n-propyl alcohol, isopropyl alcohol, n-butyl alcohol, ethylene glycol, benzyl alcohol, etc.), a petroleum fraction (petroleum ether, kerosene, solvent naphtha, etc.), aliphatic or alicyclic hydrocarbons (n-hexane, cyclohexane, etc.), aromatic hydrocarbons (benzene, toluene, xylene, ethylbenzene, chlorobenzene, cumene, methylnaphthalene, etc.), halogenated hydrocarbons (chloroform, dichloromethane, etc.), ethers (isopropyl ether, ethylene oxide, tetrahydrofuran, etc.), ketones (acetone, methyl ethyl ketone, cyclohexanone, methyl isobutyl ketone, etc.), esters (ethyl acetate, butyl acetate, ethylene glycol acetate, amyl acetate, etc.), acid amides (dimethylformamide, dimethylacetanilide, etc.), nitriles (acetonitrile, propionitrile, acrylonitrile, etc.), sulfoxides (dimethyl sulfoxide, etc.), alcohol ethers (ethylene glycol monomethyl ether, ethylene glycol monoethyl ether, etc.) or the like is exemplified.

As the adjuvant, for example, a nonionic surfactant (polyoxyethylene alkyl ether, polyoxyethylene alkyl ester, polyoxyethylene alkyl phenyl ether, polyoxyethylene sorbitan alkyl ester, sorbitan alkyl ester, etc.), an anionic surfactant (alkyl benzenesulfonate, alkyl sulfosuccinate, polyoxyethylene alkyl sulfate, aryl sulfonate, etc.), a cationic surfactant (alkylamines, polyoxyethylene alkylamines, quaternary ammonium salts, etc.), an amphoteric surfactant (alkylaminoethylglycine, alkyldimethylbetaine, etc.), polyvinyl alcohol, hydroxypropylcellulose, carboxymethylcellulose, gum arabic, tragacanth gum, xanthan gum, polyvinyl acetate, gelatin, casein, sodium alginate or the like is exemplified.

As other components, other active ingredients such as the fungicide, insecticide or miticide, nematicide, and pesticide for soil insect pests described above; a plant regulating agent, a synergist, a fertilizer, a soil improvement agent, animal feed or the like is exemplified.

The content of the heteroaryl azole compound of the present invention in the plant growth promoter differs variously depending on a preparation form, an application method, and other conditions, but is preferably 0.5 to 95% by mass, and particularly preferably in the range of 2 to 70% by mass.

The plant to which the plant growth promoter is applied is not particularly limited. For example, cereals of the family Poaceae, such as rice, barley, wheat, Japanese millet, corn, and foxtail millet; vegetables such as pumpkin, turnip, cabbage, daikon radish, Chinese cabbage, spinach, bell pepper, and tomato; flowers and ornamental plants such as chrysanthemum, gerbera, pansy, orchid, peony, and tulip; beans such as azuki bean, kidney bean, soybean, peanut, broad bean, and garden pea; tubers and roots such as potato, sweet potato, eddo, Japanese yam, and taro; Allium such as green onion, onion, and rakkyo, or the like is exemplified.

As a method for applying the plant growth promoter of the present invention, application to plants (foliage application), application to plant growing soil (soil application), application to paddy water (submerged application), application to seeds (seed treatment), or the like is exemplified.

The amount of the plant growth promoter of the present invention applied differs depending on a plant to which the plant growth promoter is applied, etc. In case of foliage application, 50 to 300 L/10 are of a solution containing 1 to 10000 ppm, preferably 10 to 1000 ppm of the active ingredient is preferably applied, and in case of soil application and submerged application, 0.1 to 1000 g/are, particularly preferably 10 to 100 g/10 are of active ingredient is preferably applied. In case of seed treatment, 0.001 to 50 g of the active ingredient per 1 kg of seeds is preferably applied.

### [Formulated Preparation]

Some formulated preparations of the pest control agent, the insecticide or miticide, the ectoparasite control agent or the endoparasite control agent or expellant of the present invention will be shown. However, additives and addition ratios should not be limited by these examples and may be changed in wide ranges. The term “part” in the formulated preparations represents part by weight.

Hereinafter, the formulated preparations for agriculture or horticulture and for paddy rice will be shown.

### (Formulation 1: Wettable Powder)

40 parts of the heteroaryl azole compound of the present invention, 53 parts of diatomaceous earth, 4 parts of higher alcohol sulfuric acid ester, and 3 parts of alkyl naphthalenesulfonate are uniformly mixed and finely milled to obtain a wettable powder containing 40% of the active ingredient.

### (Formulation 2: Emulsion)

30 parts of the heteroaryl azole compound of the present invention, 33 parts of xylene, 30 parts of dimethylformamide, and 7 parts of polyoxyethylene alkyl allyl ether are mixed and dissolved to obtain an emulsion containing 30% of the active ingredient.

### (Formulation 3: Granular Formulation)

5 parts of the heteroaryl azole compound of the present invention, 40 parts of talc, 38 parts of clay, parts of bentonite, and 7 parts of sodium alkyl sulfate are uniformly mixed, finely milled, and then granulated into a granular shape of 0.5 to 1.0 mm in diameter to obtain a granular formulation containing 5% of the active ingredient.

### (Formulation 4: Granular Formulation)

5 parts of the heteroaryl azole compound of the present invention, 73 parts of clay, 20 parts of bentonite, 1 part of dioctyl sulfosuccinate sodium salt, and 1 part of potassium phosphate are well milled and mixed, and after addition of water, the mixture is well kneaded, then granulated, and dried to obtain a granular formulation containing 5% of the active ingredient.

### (Formulation 5: Suspension)

10 parts of the heteroaryl azole compound of the present invention, 4 parts of polyoxyethylene alkyl allyl ether, 2 parts of polycarboxylic acid sodium salt, parts of glycerin, 0.2 parts of xanthan gum, and 73.8 parts of water are mixed and wet-milled until the particle size becomes 3 microns or smaller to obtain a suspension containing 10% of the active ingredient.

Hereinafter, the formulated preparations of the ectoparasite control agent or the endoparasite control agent or expellant will be shown.

### (Formulation 6: Granules)

5 parts of the heteroaryl azole compound of the present invention are dissolved in an organic solvent to obtain a solution. The solution is sprayed onto 94 parts of kaolin and 1 part of white carbon. Then, the solvent is evaporated under reduced pressure. This type of granules may be mixed with animal feed.

### (Formulation 7: Injectable Filler)

0.1 to 1 parts of the heteroaryl azole compound of the present invention and 99 to 99.9 parts of peanut oil are uniformly mixed and then sterilized by filtration through a sterilizing filter.

### (Formulation 8: Pore-on Formulation)

5 parts of the heteroaryl azole compound of the present invention, 10 parts of myristic acid ester, and parts of isopropanol are uniformly mixed to obtain a pore-on formulation.

### (Formulation 9: Spot-on Formulation)

10 to 15 parts of the heteroaryl azole compound of the present invention, 10 parts of palmitic acid ester, and 75 to 80 parts of isopropanol are uniformly mixed to obtain a spot-on formulation.

### (Formulation 10: Spray Formulation)

1 part of the heteroaryl azole compound of the present invention, 10 parts of propylene glycol, and 89 parts of isopropanol are uniformly mixed to obtain a spray formulation.

Next, the present invention will be more specifically described with reference to Examples of compounds. However, the present invention is not limited by the following Examples of compounds by any means.

### Example 1

### Synthesis of 2-(5-(ethylsulfonyl)-6-(1-methyl-5-(perfluoropropyl)-1H-imidazol-2-yl)pyridin-3-yl)pyrimidine (compound No. b-19)

### (Step 1) Synthesis of 2-bromo-5-chloro-3-(ethylthio)pyridine

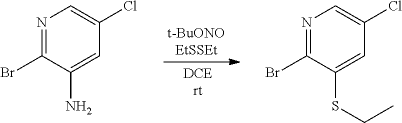

3-Amino-2-bromo-5-chloropyridine (40.0 g) and diethyl disulfide (16.5 g) were dissolved in 1,2-dichloroethane (770 ml), and the obtained solution was stirred at room temperature. t-Butyl nitrite (29.8 g) was added thereto, and the mixture was heated overnight at 50° C. The obtained solution was poured into water, followed by extraction with chloroform. The obtained organic layer was washed with saturated brine, dried over anhydrous magnesium sulfate, and filtered. The filtrate was concentrated under reduced pressure. The obtained concentrate was purified by silica gel column chromatography to obtain 30.0 g of the title compound (yield: 62%).

¹H-NMR of the obtained title compound will be shown below.

¹H-NMR (CDCl₃) δ: 8.08 (1H, d), 7.37 (1H, d), 2.96 (2H, q), 1.42 (3H, t).

### (Step 2) Synthesis of 5-chloro-3-(ethylthio)-2-(1-methyl-1H-imidazol-2-yl)pyridine

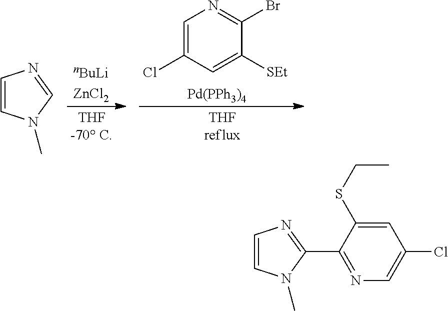

In a reaction vessel, 1-methyl-1H-imidazole (4.0 g) was dissolved in tetrahydrofuran (200 ml), and the reaction vessel was replaced with nitrogen. Then, the reaction solution was cooled to −70° C. A 2.65 M solution of n-butyllithium in n-hexane (21 ml) was added dropwise thereto, and the mixture was stirred at −70° C. for minutes. A 1 M solution of zinc chloride in tetrahydrofuran (83 ml) was added thereto, and the mixture was warmed to room temperature and stirred for 1 hour. Then, 2-bromo-5-chloro-3-(ethylthio)pyridine (10.4 g) and tetrakis(triphenylphosphine)palladium (2.4 g) were added thereto, and the reaction vessel was replaced with nitrogen. Then, the reaction mixture was stirred overnight under heating to reflux. The obtained solution was poured into an aqueous Rochelle salt solution, followed by extraction with ethyl acetate. The obtained organic layer was washed with an aqueous sodium hydroxide solution and saturated brine, dried over anhydrous magnesium sulfate, and filtered. The filtrate was concentrated under reduced pressure. The obtained concentrate was purified by silica gel column chromatography to obtain 9.65 g of the title compound (yield: 92%).

¹H-NMR of the obtained title compound will be shown below.

¹H-NMR (CDCl₃) δ: 8.31 (1H, d), 7.59 (1H, d), 7.21 (1H, d), 6.99 (1H, d), 3.85 (3H, s), 2.92 (2H, q), 1.37 (3H, t).

### (Step 3) Synthesis of 5-chloro-3-(ethylsulfonyl)-2-(1-methyl-1H-imidazol-2-yl)pyridine

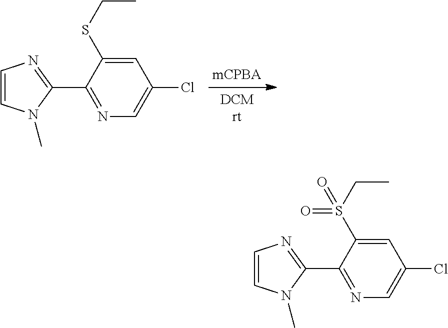 [SMILES: CCSc1cc(Cl)cnc1-c1nccn1C ; CCS(=O)(=O)c1cc(Cl)cnc1-c1nccn1C]

5-Chloro-3-(ethylthio)-2-(1-methyl-1H-imidazol-2-yl)pyridine (0.80 g) was dissolved in dichloromethane (ml), and the solution was cooled to 0° C. 70% m-chloroperbenzoic acid (1.71 g) was added thereto, and the mixture was stirred at room temperature for 5 hours. The obtained solution was poured into a mixed solution of a saturated aqueous solution of sodium bicarbonate and a saturated aqueous solution of sodium thiosulfate, followed by extraction with dichloromethane. The obtained organic layer was washed with saturated brine, dried over anhydrous magnesium sulfate, and filtered. The filtrate was concentrated under reduced pressure. The obtained concentrate was purified by silica gel column chromatography to obtain 0.84 g of the title compound (yield: 93%).

¹H-NMR of the obtained title compound will be shown below.

¹H-NMR (CDCl₃) δ: 8.83 (1H, d), 8.46 (1H, d), 7.14 (1H, d), 7.05 (1H, d), 3.93 (2H, q), 3.68 (3H, s), 1.34 (3H, t).

### (Step 4) Synthesis of 2-(5-bromo-1-methyl-1H-imidazol-2-yl)-5-chloro-3-(ethylsulfonyl)pyridine

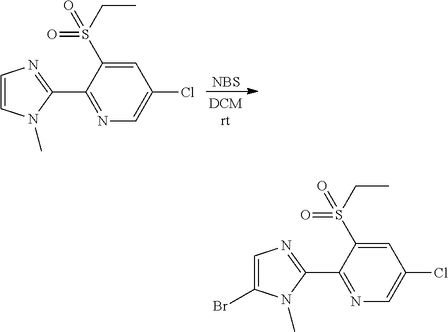

5-Chloro-3-(ethylsulfonyl)-2-(1-methyl-1H-imidazol-2-yl)pyridine (0.84 g) was dissolved in dichloromethane (30 ml), and the solution was cooled to 0° C. N-Bromosuccinimide (0.50 g) was added thereto, and the mixture was stirred at room temperature for 3 hours. The obtained solution was poured into water, followed by extraction with dichloromethane. The obtained organic layer was washed with saturated brine, dried over anhydrous magnesium sulfate, and filtered. The filtrate was concentrated under reduced pressure, and the obtained concentrate was purified by silica gel column chromatography to obtain 0.93 g of the title compound (yield: 89%).

¹H-NMR of the obtained title compound will be shown below.

¹H-NMR (CDCl₃) δ: 8.83 (1H, d), 8.46 (1H, d), 7.13 (1H, s), 3.88 (2H, q), 3.62 (3H, s), 1.35 (3H, t).

### (Step 5) Synthesis of 5-chloro-3-(ethylsulfonyl)-2-(5-iodo-1-methyl-1H-imidazol-2-yl)pyridine

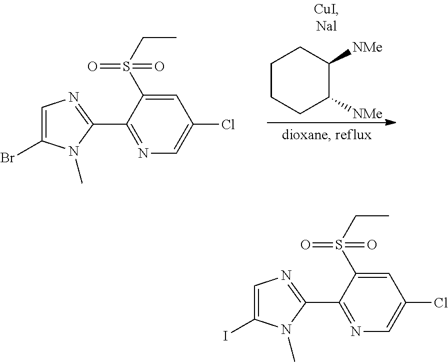

In a reaction vessel, 2-(5-bromo-1-methyl-1H-imidazol-2-yl)-5-chloro-3-(ethylsulfonyl)pyridine (0.93 g) was dissolved in dioxane (26 ml), and the reaction vessel was replaced with argon. Then, the reaction solution was stirred at room temperature. Sodium iodide (1.92 g), copper iodide (0.1 g), and trans-N,N′-dimethylcyclohexane-1,2-diamine (0.15 g) were added thereto, and the mixture was stirred overnight under heating to reflux. The obtained solution was poured into water, followed by extraction with dichloromethane. The obtained organic layer was washed with saturated brine, dried over anhydrous magnesium sulfate, and filtered. The filtrate was concentrated under reduced pressure, and the obtained concentrate was purified by silica gel column chromatography to obtain 0.93 g of the title compound (yield: 89%).

¹H-NMR of the obtained title compound will be shown below.

¹H-NMR (CDCl₃) δ: 8.84 (1H, d), 8.46 (1H, d), 7.23 (1H, s), 3.87 (2H, q), 3.62 (3H, s), 1.34 (3H, t).

### (Step 6) Synthesis of 5-chloro-3-(ethylsulfonyl)-2-(1-methyl-5-(perfluoropropyl)-1H-imidazol-2-yl)pyridine

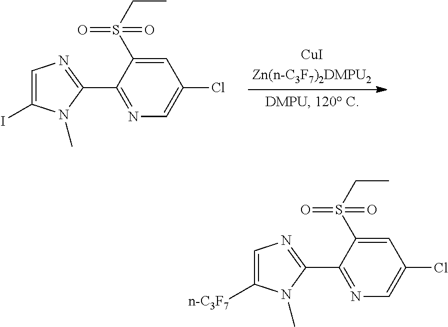

In a reaction vessel, 5-chloro-3-(ethylsulfonyl)-2-(5-iodo-1-methyl-1H-imidazol-2-yl)pyridine (0.68 g) was dissolved in N,N′-dimethylpropyleneurea (3 ml), and the reaction vessel was replaced with argon. Then, the reaction solution was stirred at room temperature. Copper iodide (0.57 g) and bis(n-perfluoropropyl)zinc/DMPU complex (1.96 g) were added thereto, and the mixture was stirred overnight at 120° C. The obtained solution was poured into a saturated aqueous solution of Rochelle salt, followed by extraction with ethyl acetate. The obtained organic layer was washed with water and saturated brine, dried over anhydrous magnesium sulfate, and filtered. The filtrate was concentrated under reduced pressure, and the obtained concentrate was purified by silica gel column chromatography to obtain 0.61 g of the title compound (yield: 82%).

¹H-NMR of the obtained title compound will be shown below.

¹H-NMR (CDCl₃) δ: 8.89 (1H, d), 8.47 (1H, d), 7.50 (1H, s), 3.73 (2H, q), 3.65 (3H, s), 1.34 (3H, t).

### (Step 7) Synthesis of 2-(5-(ethylsulfonyl)-6-(1-methyl-5-(perfluoropropyl)-1H-imidazol-2-yl)pyridin-3-yl)pyrimidine

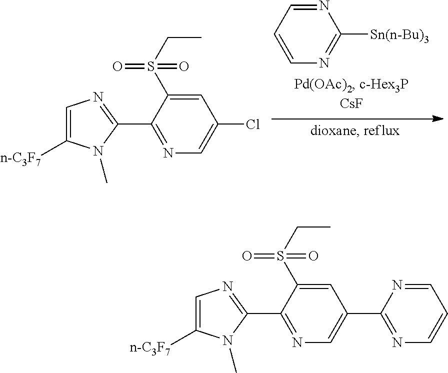

In a reaction vessel, 5-chloro-3-(ethylsulfonyl)-2-(1-methyl-5-(perfluoropropyl)-1H-imidazol-2-yl)pyridine (0.29 g) was dissolved in dioxane (10 ml), and the reaction vessel was replaced with nitrogen. Then, the reaction solution was stirred at room temperature. 2-(Tributylstannyl)pyrimidine (0.35 g), palladium(II) acetate (0.03 g), a solution of 18% tricyclohexylphosphine in toluene (0.36 g), and cesium fluoride (0.22 g) were added thereto, and the mixture was stirred overnight under heating to reflux. The obtained solution was poured into a 10% aqueous potassium fluoride solution, followed by extraction with ethyl acetate. The obtained organic layer was washed with water and saturated brine, dried over anhydrous magnesium sulfate, and filtered. The filtrate was concentrated under reduced pressure, and the obtained concentrate was purified by silica gel column chromatography to obtain 0.10 g of the title compound (yield: 31%).

¹H-NMR and ¹⁹F-NMR of the obtained title compound will be shown below.

¹H-NMR (CDCl₃) δ: 9.94 (1H, d), 9.48 (1H, d), 8.92 (2H, d), 7.52 (1H, s), 7.38 (1H, t), 3.75 (2H, q), 3.70 (3H, s), 1.37 (3H, t); ¹⁹F-NMR (376 MHz, CDCl₃—C₆F₆) δ −80.41 (3H, t) −107.20 (2H, q), −125.60-−125.68 (2H, m).

### Example 2

### Synthesis of 5-(ethylsulfonyl)-2-(1-methyl-1H-1,2,4-triazol-3-yl)-4-(1-methyl-5-(perfluorobutyl)-1H-imidazol-2-yl)pyrimidine (compound No. a-2)

### (Step 1) Synthesis of 2-chloro-1-(5-iodo-1-methyl-1H-imidazol-2-yl)ethan-1-one

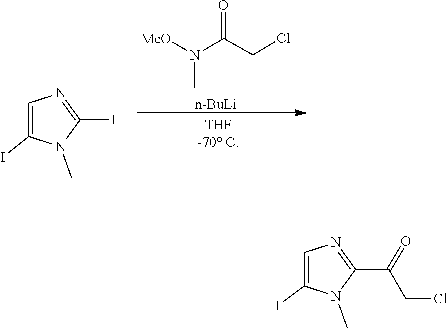

In a reaction vessel, 2,5-diiodo-1-methyl-1H-imidazole (34.6 g) was dissolved in tetrahydrofuran (1130 ml), and the reaction vessel was replaced with nitrogen. Then, the reaction solution was cooled to −70° C. n-Butyllithium (2.65 M, a solution in n-hexane, 42 ml) was added dropwise thereto, and the mixture was stirred at −70° C. for 30 minutes. 2-Chloro-N-methoxy-N-methylacetamide (17.0 g) was added thereto, and the mixture was stirred at −70° C. for 1 hour. The obtained solution was poured into a saturated aqueous solution of ammonium chloride, followed by extraction with ethyl acetate. The obtained organic layer was washed with saturated brine, dried over anhydrous magnesium sulfate, and filtered. The filtrate was concentrated under reduced pressure to obtain a concentrate.

### (Step 2) Synthesis of 2-(ethylthio)-1-(5-iodo-1-methyl-1H-imidazol-2-yl)ethan-1-one

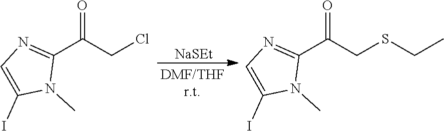

The concentrate obtained in step 1 (unpurified 2-chloro-1-(5-iodo-1-methyl-1H-imidazol-2-yl)ethan-1-one) was dissolved in a mixed solvent of N,N-dimethylformamide (100 ml) and tetrahydrofuran (100 ml), and the obtained solution was stirred at 0° C. Sodium ethyl mercaptan (10.0 g, 80%) was added thereto, and the mixture was stirred overnight at room temperature. The obtained solution was poured to water, followed by extraction with ethyl acetate. The obtained organic layer was washed with saturated brine, dried over anhydrous magnesium sulfate, and filtered. The filtrate was concentrated under reduced pressure, and the concentrate was purified by silica gel column chromatography to obtain 5.74 g of the title compound (yield: 18%, 2 steps).

¹H-NMR of the obtained title compound will be shown below.

¹H-NMR (400 MHz, CDCl₃): δ 7.28 (1H, s), 4.01 (3H, s), 3.94 (2H, s), 2.64 (2H, q), 1.28 (3H, t).

### (Step 3) Synthesis of 2-(ethylsulfonyl)-1-(5-iodo-1-methyl-1H-imidazol-2-yl)ethan-1-one

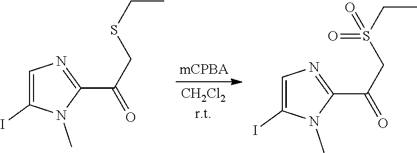

2-(Ethylthio)-1-(5-iodo-1-methyl-1H-imidazol-2-yl)ethan-1-one (5.7 g) was dissolved in dichloromethane (190 ml), and the obtained solution was stirred at 0° C. m-Chloroperbenzoic acid (70%, 10 g) was added thereto, and the mixture was stirred at room temperature for 5 hours. The obtained solution was poured into a mixed solution of a saturated aqueous solution of sodium bicarbonate and a saturated aqueous solution of sodium thiosulfate, followed by extraction with dichloromethane. The obtained organic layer was washed with saturated brine, dried over anhydrous magnesium sulfate, and filtered. The filtrate was concentrated under reduced pressure, and the concentrate was purified by silica gel column chromatography to obtain 5.2 g of the title compound (yield: 81%).

¹H-NMR of the obtained title compound will be shown below.

¹H-NMR (400 MHz, CDCl₃): δ 7.35 (1H, s), 4.81 (2H, s), 4.02 (3H, s), 3.28 (2H, q), 1.46 (3H, t).

### (Step 4) Synthesis of 3-(dimethylamino)-2-(ethylsulfonyl)-1-(5-iodo-1-methyl-1H-imidazol-2-yl)prop-2-en-1-one

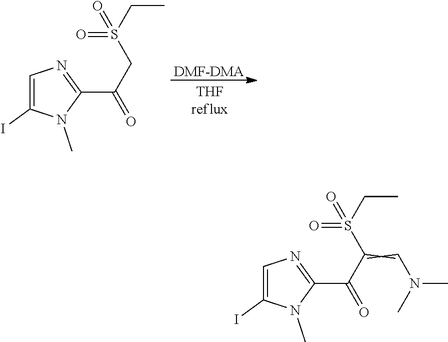

2-(Ethylsulfonyl)-1-(5-iodo-1-methyl-1H-imidazol-2-yl)ethan-1-one (0.34 g) was dissolved in tetrahydrofuran (5 ml), and the obtained solution was stirred at room temperature. N,N-Dimethylformamide dimethyl acetal (0.23 g) was added thereto, and the mixture was stirred under heating to reflux for 4 hours. The obtained solution was concentrated under reduced pressure.

### (Step 5) Synthesis of 5-(ethylsulfonyl)-4-(5-iodo-1-methyl-1H-imidazol-2-yl)-2-(1-methyl-1H-1,2,4-triazol-3-yl)pyrimidine

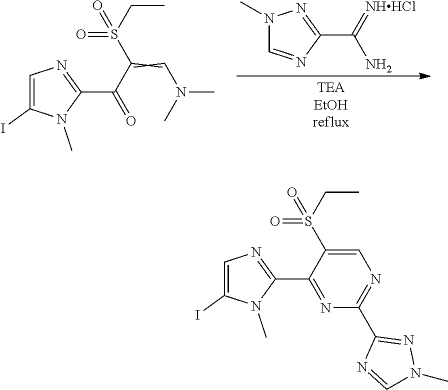

The concentrate obtained in step 4 (unpurified 3-(dimethylamino)-2-(ethylsulfonyl)-1-(5-iodo-1-methyl-1H-imidazol-2-yl)prop-2-en-1-one) was dissolved in ethanol (10 ml), and the solution was stirred at room temperature. Triethylamine (0.30 g) and 1-methyl-1H-1,2,4-triazole-3-carboximidamide hydrochloride (0.20 g) were added thereto, and the mixture was stirred under heating to reflux for 5 hours. The obtained solution was poured into a saturated aqueous solution of ammonium chloride, followed by extraction with dichloromethane. The obtained organic layer was washed with saturated brine, dried over anhydrous magnesium sulfate, and filtered. The filtrate was concentrated under reduced pressure, and the obtained concentrate was purified by silica gel column chromatography to obtain 0.44 g of the title compound (yield: 95%, 2 steps).

¹H-NMR of the obtained title compound will be shown below.

¹H-NMR (400 MHz, CDCl₃): δ 9.50 (1H, s), 8.25 (1H, s), 7.31 (1H, s), 4.11 (3H, s), 4.07 (2H, q), 3.94 (3H, s), 1.41 (3H, t).

### (Step 6) Synthesis of 5-(ethylsulfonyl)-2-(1-methyl-1H-1,2,4-triazol-3-yl)-4-(1-methyl-5-(perfluorobutyl)-1H-imidazol-2-yl)pyrimidine

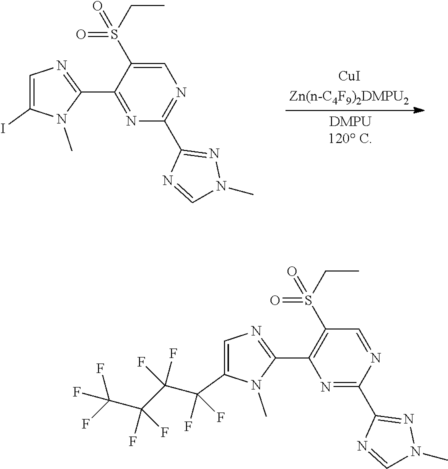 [SMILES: CCS(=O)(=O)c1cnc(-c2ncn(C)n2)nc1-c1ncc(I)n1C ; CCS(=O)(=O)c1cnc(-c2ncn(C)n2)nc1-c1ncc(C(F)(F)C(F)(F)C(F)(F)C(F)(F)F)n1C]

In a reaction vessel, 5-(ethylsulfonyl)-4-(5-iodo-1-methyl-1H-imidazol-2-yl)-2-(1-methyl-1H-1,2,4,-triazol-3-yl)pyrimidine (0.2 g) was dissolved in N,N′-dimethylpropyleneurea (3 ml), and the reaction vessel was replaced with argon and then stirred at room temperature. Copper iodide (0.17 g) and bis(n-perfluorobutyl)zinc/DMPU complex (0.66 g) synthesized with reference to the method described in Chem. Eur. J. 2015, 21, 96-100 were added thereto, and the mixture was stirred at 110° C. for 3 hours. The obtained solution was poured into a saturated aqueous solution of Rochelle salt, followed by extraction with ethyl acetate. The obtained organic layer was washed with water and saturated brine, dried over anhydrous magnesium sulfate, and filtered. The filtrate was concentrated under reduced pressure, and the obtained concentrate was purified by silica gel column chromatography to obtain 0.06 g of the title compound (yield: 25%).

¹H-NMR and ¹⁹F-NMR of the obtained title compound will be shown below.

¹H-NMR (400 MHz, CDCl₃): δ 9.53 (1H, s), 8.27 (1H, s), 7.54 (1H, s), 4.12 (3H, s), 3.89 (3H, s), 3.88 (2H, q), 1.40 (3H, t); ¹⁹F-NMR (376 MHz, CDCl₃—C₆F₆): δ −81.3 (s, 3F), −106.3(s, 2F), −121.8 (s, 2F), −126.0 (s, 2F).

### Example 3

### Synthesis of 5-(ethylsulfonyl)-4-(4-methyl-5-(nonafluorobutyl)-4H-1,2,4-triazol-3-yl)-2,2′-bipyrimidine (compound No. a-5)

### (Step 1) Synthesis of 2,2,3,3,4,4,5,5,5-nonafluoropentanehydrazide

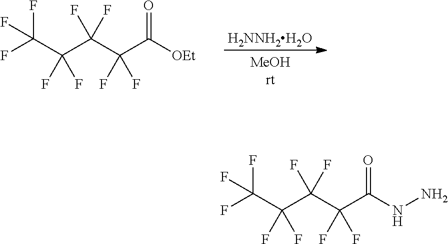

Ethyl 2,2,3,3,4,4,5,5,5-nonafluoropentanoate (15.8 g) was dissolved in methanol (108 ml), and the solution was stirred at room temperature. Hydrazine monohydrate (2.7 g) was added dropwise thereto, and the mixture was stirred at room temperature for 30 minutes. The obtained solution was concentrated under reduced pressure.

### (Step 2) Synthesis of N-methyl-2-(2,2,3,3,4,4,5,5,5-nonafluoropentanoyl)hydrazine-1-carbothioamide

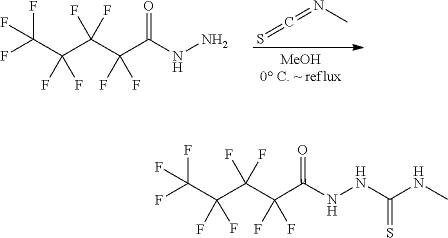

16.4 g of the concentrate obtained in step 1 (unpurified 2,2,3,3,4,4,5,5,5-nonafluoropentanehydrazide) was dissolved in methanol (54 ml), and the solution was stirred at 0° C. A solution of methyl isothiocyanate (3.95 g) in methanol (54 mL) was added dropwise thereto, and the mixture was stirred at 0° C. for 10 minutes. Then, the reaction mixture was stirred under heating to reflux for 3 hours. The obtained solution was allowed to cool to room temperature and then concentrated under reduced pressure.

### (Step 3) Synthesis of 4-methyl-5-(nonafluorobutyl)-2,4-dihydro-3H-1,2,4-triazole-3-thione

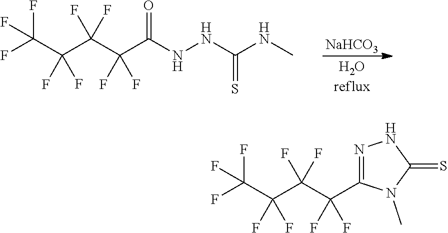

23.5 g of the concentrate (containing unpurified N-methyl-2-(2,2,3,3,4,4,5,5,5-nonafluoropentanoyl)hydrazine-1-carbothioamide) obtained in step 2 was suspended in water (270 ml), and the suspension was stirred at room temperature. Sodium bicarbonate (45.5 g) was added thereto, and the mixture was stirred under heating to reflux for 4 hours. The obtained solution was allowed to cool to room temperature and then adjusted to pH 2 with concentrated hydrochloric acid, followed by extraction with ethyl acetate. The obtained organic layer was washed with saturated brine, dried over anhydrous magnesium sulfate, and filtered. The filtrate was concentrated under reduced pressure.

### (Step 4) Synthesis of 4-methyl-3-(nonafluorobutyl)-4H-1,2,4-triazole

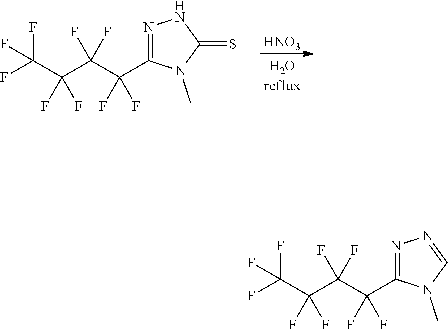

15.7 g of the concentrate (containing 4-methyl-5-(nonafluorobutyl)-2,4-dihydro-3H-1,2,4-triazole-3-thione) obtained in step 3 was suspended in water (424 ml), and the suspension was stirred at room temperature. Nitric acid (47 ml) was added thereto, and the mixture was stirred under heating to reflux for 4 hours. The obtained solution was allowed to cool to room temperature and then adjusted to pH 12 with a 30% aqueous sodium hydroxide solution under cooling in an ice bath, followed by extraction with chloroform. The obtained organic layer was dried over anhydrous magnesium sulfate and filtered. The filtrate was concentrated under reduced pressure, and the obtained concentrate was purified by silica gel column chromatography to obtain 10.5 g of the title compound (yield: 75%, 4 steps).

¹H-NMR of the obtained title compound will be shown below.

¹H-NMR (400 MHz, CDCl₃): δ 8.25 (s, 1H), 3.86 (s, 3H).

### (Step 5) Synthesis of 3-bromo-4-methyl-5-(nonafluorobutyl)-4H-1,2,4-triazole

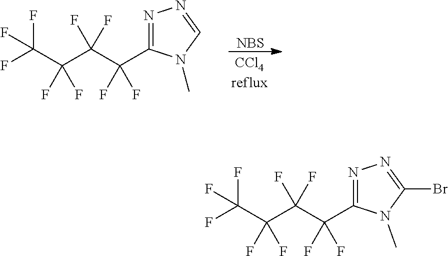

4-Methyl-3-(nonafluorobutyl)-4H-1,2,4-triazole (3.44 g) was dissolved in carbon tetrachloride (114 mL), and the solution was stirred at room temperature. N-Bromosuccinimide (2.24 g) was added thereto, and the mixture was stirred under heating to reflux for 20 hours. The obtained solution was allowed to cool to room temperature and then concentrated under reduced pressure. The obtained concentrate was purified by silica gel column chromatography to obtain 2.49 g of the title compound (yield: 57%).

¹H-NMR of the obtained title compound will be shown below.

¹H-NMR (400 MHz, CDCl₃): δ 3.79 (s, 3H).

### (Step 6) Synthesis of 2-chloro-1-(4-methyl-5-(nonafluorobutyl)-4H-1,2,4-triazol-3-yl)ethan-1-one

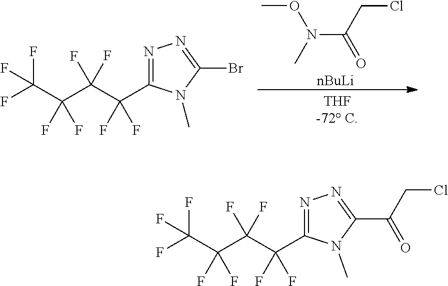

3-Bromo-4-methyl-5-(nonafluorobutyl)-4H-1,2,4-triazole (1.50 g) was dissolved in tetrahydrofuran (16 ml), and the reaction system was replaced with nitrogen and then cooled to −72° C. n-Butyllithium (2.76 M, a solution in n-hexane, 1.70 ml) was added dropwise thereto, and the mixture was stirred at −72° C. for 30 minutes. A solution of 2-chloro-N-methoxy-N-methylacetamide (0.652 g) in tetrahydrofuran (4 ml) was added dropwise thereto, and the mixture was stirred at −72° C. for 1 hour. The obtained solution was poured into a saturated aqueous solution of ammonium chloride, followed by extraction with ethyl acetate. The obtained organic layer was washed with saturated brine, dried over anhydrous magnesium sulfate, and filtered. The filtrate was concentrated under reduced pressure, and the obtained concentrate was purified by silica gel column chromatography to obtain 0.90 g of the title compound (yield: 60%).

¹H-NMR of the obtained title compound will be shown below.

¹H-NMR (400 MHz, CDCl₃): δ 5.04 (s, 2H), 4.13 (s, 3H).

### (Step 7) Synthesis of 2-(ethylthio)-1-(4-methyl-5-(nonafluorobutyl)-4H-1,2,4-triazol-3-yl)ethan-1-one

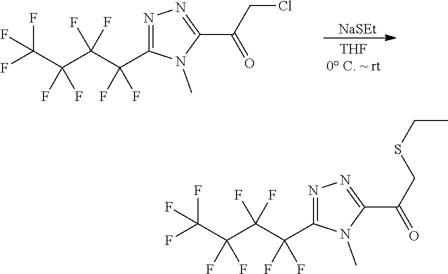 [SMILES: Cn1c(C(=O)CCl)nnc1C(F)(F)C(F)(F)C(F)(F)C(F)(F)F ; CCSCC(=O)c1nnc(C(F)(F)C(F)(F)C(F)(F)C(F)(F)F)n1C ; "NaSEt"]

2-Chloro-1-(4-methyl-5-(nonafluorobutyl)-4H-1,2,4-triazol-3-yl)ethan-1-one (0.90 g) was dissolved in tetrahydrofuran (24 ml), and the solution was stirred at room temperature. Sodium ethyl mercaptan (90%, 0.245 g) was added thereto, and the mixture was stirred at room temperature for 6 hours. Sodium ethyl mercaptan (90%, 0.245 g) was further added thereto, and the mixture was stirred at room temperature for 1 hour. The obtained solution was poured into a saturated aqueous solution of ammonium chloride, followed by extraction with ethyl acetate. The obtained organic layer was washed with saturated brine, dried over anhydrous magnesium sulfate, and filtered. The filtrate was concentrated under reduced pressure.

### (Step 8) Synthesis of 2-(ethylsulfonyl)-1-(4-methyl-5-(nonafluorobutyl)-4H-1,2,4-triazol-3-yl)ethan-1-one

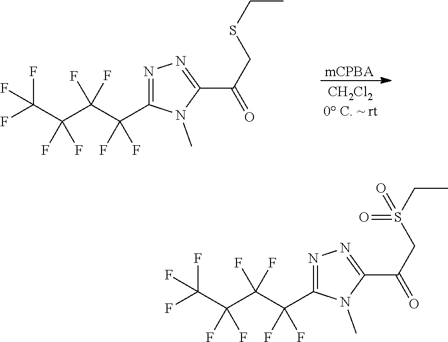 [SMILES: CCSCC(=O)c1nnc(C(F)(F)C(F)(F)C(F)(F)C(F)(F)F)n1C ; "mCPBA" ; CCS(=O)(=O)CC(=O)c1nnc(C(F)(F)C(F)(F)C(F)(F)C(F)(F)F)n1C]

0.98 g of the concentrate (containing 2-(ethylthio)-1-(4-methyl-5-(nonafluorobutyl)-4H-1,2,4-triazol-3-yl)ethan-1-one) obtained in step 7 was dissolved in dichloromethane (24 ml), and the solution was stirred at 0° C. m-Chloroperbenzoic acid (70%, 1.32 g) was added thereto, and the mixture was stirred at room temperature for 3 hours. The obtained solution was poured into a mixed solution of a saturated aqueous solution of sodium bicarbonate and a saturated aqueous solution of sodium thiosulfate, followed by extraction with chloroform. The obtained organic layer was dried over anhydrous magnesium sulfate and filtered. The filtrate was concentrated under reduced pressure. The obtained concentrate was purified by silica gel column chromatography to obtain 0.807 g of the title compound (yield: 78%, 2 steps).

¹H-NMR of the obtained title compound will be shown below.

¹H-NMR (400 MHz, CDCl₃): δ 4.92 (s, 2H), 4.11 (s, 3H), 3.31 (q, 2H), 1.48 (t, 3H).

### (Step 9) Synthesis of 3-(dimethylamino)-2-(ethylsulfonyl)-1-(4-methyl-5-(nonafluorobutyl)-4H-1,2,4-triazol-3-yl)prop-2-en-1-one

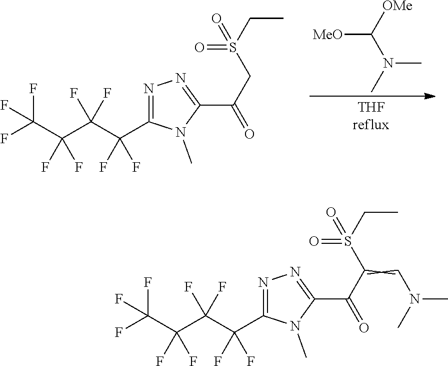

2-(Ethylsulfonyl)-1-(4-methyl-5-(nonafluorobutyl)-4H-1,2,4-triazol-3-yl)ethan-1-one (0.20 g) was dissolved in tetrahydrofuran (2.3 ml), and the solution was stirred at room temperature. N,N-Dimethylformamide dimethyl acetal (0.274 g) was added thereto, and the mixture was stirred under heating to reflux for 1.5 hours. The obtained solution was concentrated under reduced pressure.

### (Step 10) Synthesis of 5-(ethylsulfonyl)-4-(4-methyl-5-(nonafluorobutyl)-4H-1,2,4-triazol-3-yl)-2,2′-bipyrimidine

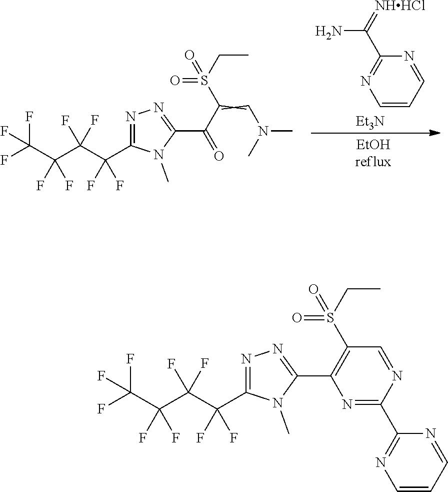

0.27 g of the concentrate (containing 3-(dimethylamino)-2-(ethylsulfonyl)-1-(4-methyl-5-(nonafluorobutyl)-4H-1,2,4-triazol-3-yl)prop-2-en-1-one) obtained in step 9 was dissolved in ethanol (2.3 ml), and the solution was stirred at room temperature. Triethylamine (0.209 g) and 2-amidinopyrimidine hydrochloride (0.109 g) were added thereto, and the mixture was stirred under heating to reflux for 1 hour. The obtained solution was poured into a saturated aqueous solution of ammonium chloride, followed by extraction with chloroform. The obtained organic layer was dried over anhydrous magnesium sulfate and filtered. The filtrate was concentrated under reduced pressure, and the obtained concentrate was purified by silica gel column chromatography to obtain 0.10 g of the title compound (yield: 40%, 2 steps).

¹H-NMR and ¹⁹F-NMR of the obtained title compound will be shown below.

¹H-NMR (400 MHz, CDCl₃) δ 9.70 (s, 1H), 9.09 (d, 2H), 7.56 (t, 1H), 3.92 (s, 3H), 3.83 (q, 2H), 1.42 (t, 3H); ¹⁹F-NMR (376 MHz, CDCl₃—C₆F₆) δ −81.28 (t, 3F), −109.73 (t, 2F), −122.00-−122.09 (m, 2F), −125.72-−125.81 (m, 2F).

Some compounds of the present invention produced in the same way as in Examples described above are shown in Tables 1 to 3. Table 1 shows the substituents of the compound represented by the formula (I). In the tables, properties, melting point (m.p.), or refractive index (n_{D}) are also shown as the physical properties of each compound.

In the tables, Me represents a methyl group, Et represents an ethyl group, ^{c}Pr represents a cyclopropyl group, and Ac represents an acetyl group.

TABLE 1

| Compound | | |
|---|---|---|
| No. | Structure | Physical properties |
| a-1 | 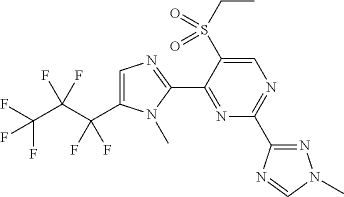 [SMILES: CCS(=O)(=O)c1cnc(-c2ncn(C)n2)nc1-c1ncc(C(F)(F)C(F)(F)C(F)(F)F)n1C] | m.p.: 186-188(° C.) |
| a-2 | 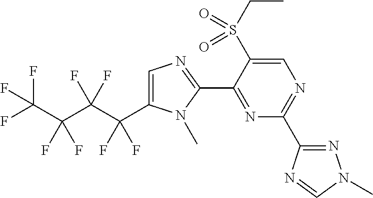 [SMILES: CCS(=O)(=O)c1cnc(-c2ncn(C)n2)nc1-c1ncc(C(F)(F)C(F)(F)C(F)(F)C(F)(F)F)n1C] | m.p.: 188-190(° C.) |
| a-3 | 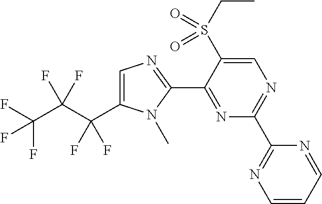 [SMILES: CCS(=O)(=O)c1cnc(-c2ncccn2)nc1-c1ncc(C(F)(F)C(F)(F)C(F)(F)F)n1C] | m.p.: 207-209(° C.) |
| a-4 | 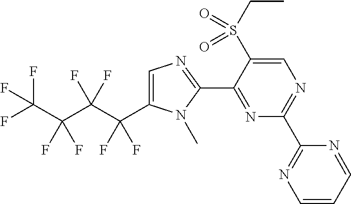 [SMILES: CCS(=O)(=O)c1cnc(-c2ncccn2)nc1-c1ncc(C(F)(F)C(F)(F)C(F)(F)C(F)(F)F)n1C] | m.p.: 208-210(° C.) |
| a-5 | 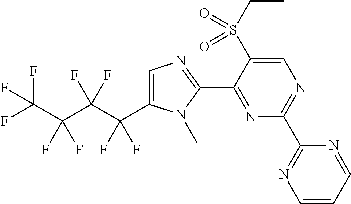 [SMILES: CCS(=O)(=O)c1cnc(-c2ncccn2)nc1-c1ncc(C(F)(F)C(F)(F)C(F)(F)C(F)(F)F)n1C] | m.p.: 172-174(° C.) |
| a-6 | 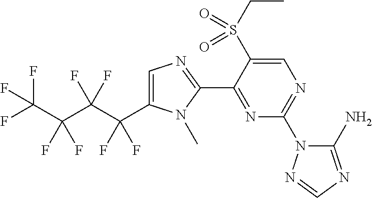 [SMILES: CCS(=O)(=O)c1cnc(-n2ncnc2N)nc1-c1ncc(C(F)(F)C(F)(F)C(F)(F)C(F)(F)F)n1C] | m.p.: 208-210(° C.) |
| a-7 | 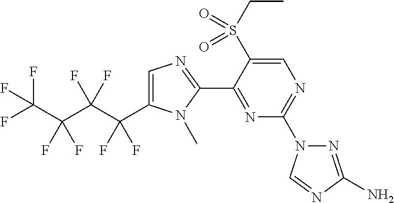 [SMILES: CCS(=O)(=O)c1cnc(-n2cnc(N)n2)nc1-c1ncc(C(F)(F)C(F)(F)C(F)(F)C(F)(F)F)n1C] | m.p.: 219-221(° C.) |
| a-8 | 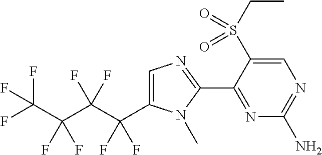 [SMILES: CCS(=O)(=O)c1cnc(N)nc1-c1ncc(C(F)(F)C(F)(F)C(F)(F)C(F)(F)F)n1C] | m.p.: 184-186(° C.) |
| a-9 | 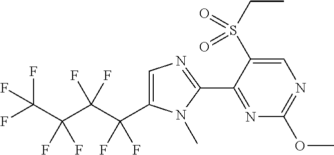 [SMILES: CCS(=O)(=O)c1cnc(OC)nc1-c1ncc(C(F)(F)C(F)(F)C(F)(F)C(F)(F)F)n1C] | m.p.: 86-88(° C.) |
| a-10 | 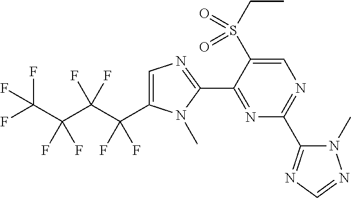 [SMILES: CCS(=O)(=O)c1cnc(-c2ncnn2C)nc1-c1ncc(C(F)(F)C(F)(F)C(F)(F)C(F)(F)F)n1C] | amorhous |

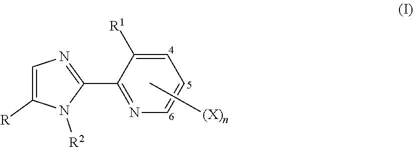

TABLE 2

| Compound | | | | | |
|---|---|---|---|---|---|
| No. | (X)ₙ | R¹ | R² | R | Physical properties |
| b-1 | 5-°Pr | SO₂Et | Me | CF₂CF₂CF₂CF₃ | m.p.: 138-140 (° C.) |
| b-2 | — | SEt | Me | CF₂CF₂CF₂CF₃ | nd(20.8° C.): 1.4817 |
| b-3 | — | SO₂Et | Me | CF₂CF₂CF₂CF₃ | m.p.: 83-85 (° C.) |
| b-4 | 5-(pyrimidin-2-yl) | SO₂Et | Me | CF₂CF₂CF₂CF₃ | m.p.: 114-116 (° C.) |
| b-5 | 6-(1H-1,2,4-triazol-1-yl) | SO₂Et | Me | CH(OAc)CF₂CF₂CF₂CF₃ | m.p.: 183-185 (° C.) |
| b-6 | 6-(1H-1,2,4-triazol-1-yl) | SO₂Et | Me | CH(OH)CF₂CF₂CF₂CF₃ | m.p.: 179-181 (° C.) |
| b-7 | 5-CF₃ | SO₂Et | Me | CF₂CF₂CF₂CF₃ | m.p.: 88-90 (° C.) |
| b-8 | 6-(1H-1,2,4-triazol-1-yl) | SO₂Et | Me | CF₂CF₂CF₂CF₃ | m.p.: 129-131 (° C.) |
| b-9 | 6-(pyrimidin-2-yl) | SO₂Et | Me | CF₂CF₂CF₂CF₃ | m.p.: 125-127 (° C.) |
| b-10 | 6-(1H-1,2,4-triazol-1-yl) | SO₂Et | Me | CHFCF₂CF₂CF₂CF₃ | m.p.: 143-145 (° C.) |
| b-11 | — | SO₂Et | Me | CHFCF₂CF₂CF₂CF₃ | m.p.: 78-80 (° C.) |
| b-12 | 6-NH₂ | SO₂Et | Me | CF₂CF₂CF₂CF₃ | m.p.: 53-55 (° C.) |
| b-13 | 6-(1H-1,2,4-triazol-1-yl) | SO₂Et | Me | CCIHCF₂CF₂CF₂CF₃ | m.p.: 162-164 (° C.) |
| b-14 | — | SO₂Et | Me | CCIHCF₂CF₂CF₂CF₃ | m.p.: 94-96 (° C.) |
| b-15 | 5-CF₃ | SO₂Et | Me | CF₂CF₂CF₃ | m.p.: 109-110 (° C.) |
| b-16 | 6-(1H-1,2,4-triazol-1-yl) | SO₂Et | Me | CF₂CF₂CF₃ | m.p.: 147-149 (° C.) |
| b-17 | 6-(1H-1,2,4-triazol-1-yl) | SO₂Et | Me | CF₂CF₃ | m.p.: 151-152 (° C.) |
| b-18 | 5-°Pr | SO₂Et | Me | CHFCF₂CF₂CF₃ | m.p.: 106-108 (° C.) |
| b-19 | 5-(pyrimidin-2-yl) | SO₂Et | Me | CF₂CF₂CF₃ | m.p.: 133-135 (° C.) |
| b-20 | 5-°Pr | SO₂Et | Me | CF₂CF₂CF₃ | m.p.: 118-120 (° C.) |
| b-21 | 5-(pyrimidin-2-yl) | SO₂Et | Me | CHFCF₂CF₂CF₃ | m.p.: 164-167 (° C.) |
| b-22 | 5-(pyrimidin-2-yl) | SO₂Et | Me | CF₂CF₃ | m.p.: 148-150 (° C.) |
| b-23 | 5-Br | SO₂Et | Me | CHFCF₂CF₃ | m.p.: 177-180 (° C.) |
| b-24 | 5-(pyrimidin-2-yl) | SO₂Et | Me | CHFCF₂CF₃ | m.p.: 170-173 (° C.) |
| b-25 | 5-°Pr | SO₂Et | Me | CHFCF₂CF₃ | m.p.: 130-132 (° C.) |
| b-26 | 5-°Pr | SO₂Et | Me | CF₂CF₃ | m.p.: 148-150 (° C.) |
| b-27 | 5-(pyridin-2-yl) | SO₂Et | Me | CHFCF₂CF₃ | m.p.: 128-131 (° C.) |
| b-28 | 5-(1H-1,2,4-triazol-1-yl) | SO₂Et | Me | CHFCF₂CF₃ | amorphous |
| b-29 | 5-(pyridin-2-yl) | SO₂Et | Me | CF₂CF₂CF₃ | m.p,: 116-118 (° C.) |
| b-30 | 5-(1-CN-2-OEt-2-oxoethyl) | SO₂Et | Me | CF₂CF₂CF₃ | m.p.: 164-166 (° C.) |
| b-31 | 5-(pyrimidin-2-yl) | SO₂Et | Me | CHFCF(CF₃)₂ | white solid |
| b-32 | 5-(pyrimidin-2-yl) | SO₂Et | Me | CHCICF₂CF₃ | m.p.: 149-151 (° C.) |
| b-33 | 5-(thiazol-2-yl) | SO₂Et | Me | CHFCF₂CF₃ | m.p.: 138-141 (° C.) |
| b-34 | 5-(3,5-F₂-phenyl) | SO₂Et | Me | CHFCF₂CF₃ | m.p.: 134-136 (° C.) |
| b-35 | 5-(pyrimidin-5-yl) | SO₂Et | Me | CHFCF₂CF₃ | m.p.: 128-131 (° C.) |
| b-36 | 5-(3-CF₃-1H-pyrazol-1-yl) | SO₂Et | Me | CF₂CF₂CF₃ | m.p.: 111-113 (° C.) |
| b-37 | 5-(pyridin-2-yloxy) | SO₂Et | Me | CF₂CF₂CF₃ | viscous oil |
| b-38 | 5-(2-oxopyridin-1(2H)-yl) | SO₂Et | Me | CF₂CF₂CF₃ | m.p.: 147-150 (° C.) |
| b-39 | 5-vinyl | SO₂Et | Me | CHFCF₂CF₃ | m.p.: 128-130 (° C.) |
| b-40 | 5-formyl | SO₂Et | Me | CHFCF₂CF₃ | viscous oil |
| b-41 | 5-(NHCO°Pr) | SO₂Et | Me | CF₂CF₂CF₃ | m.p.: 215-217 (° C.) |
| b-42 | 5-(2-CN-propan-2-yl) | SO₂Et | Me | CF₂CF₂CF₃ | m.p.: 138-140 (° C.) |
| b-43 | 5-(pyridin-2-yl) | SO₂Et | Me | CHFCF(CF₃)₂ | m.p.: 141-143 (° C.) |
| b-44 | 5-(pyridin-2-yl) | SO₂Et | Me | CHCICF(CF₃)₂ | m.p.: 160-162 (° C.) |
| b-45 | 5-(1H-1,2,4-triazol-1-yl) | SO₂Et | Me | CF₂CF₂CF₃ | m.p.: 145-147 (° C.) |
| b-46 | 6-(pyrimidin-2-yl) | SO₂Et | Me | CHFCF(CF₃)₂ | m.p.: 145-147 (° C.) |
| b-47 | 6-(1H-1,2,4-triazol-1-yl) | SO₂Et | Me | CHFCF(CF₃)₂ | m.p.: 141-143 (° C.) |
| b-48 | 5-(pyrimidin-2-yl) | SO₂Et | Me | CH₂CH(CF₃)₂ | m.p.: 184-187 (° C.) |
| b-49 | 5-CF₃ | SO₂Et | Me | CHFCF(CF₃)₂ | m.p.: 122-124 (° C.) |
| b-50 | — | SO₂Et | Me | CHFCF(CF₃)₂ | m.p.: 94-97 (° C.) |
| b-51 | 5-(1-CN-°Pr) | SO₂Et | Me | CF₂CF₂CF₃ | m.p.: 158-160 (° C.) |
| b-52 | 5-°Pr | SO₂Et | Me | CHFCF(CF₃)₂ | m.p.: 125-127 (° C.) |
| b-53 | 5-°Pr | SO₂Et | Me | CHCICF(CF₃)₂ | m.p.: 123-125 (° C.) |
| b-54 | 5-(3-CN-pyridin-2-yl) | SO₂Et | Me | CF₂CF₂CF₃ | m.p.: 118-120 (° C.) |
| b-55 | 5-(3-F-pyridin-2-yl) | SO₂Et | Me | CF₂CF₂CF₃ | m.p.: 161-163 (° C.) |
| b-56 | 5-(1-CN-°Pr) | SO₂Et | Me | CHFCF(CF₃)₂ | amorphous |
| b-57 | 5-(1-(pyridin-2-yl)-°Pr) | SO₂Et | Me | CHFCF(CF₃)₂ | m.p.: 71-75 (° C.) |
| b-58 | 5-(2-F-phenyl) | SO₂Et | Me | CF₂CF₂CF₃ | m.p.: 128-130 (° C.) |
| b-59 | 5-(pyridin-2-yl) | SO₂Et | Me | CF₂CF₂CF₂CF₃ | m.p.: 90-92 (° C.) |
| b-60 | 5-(3,5-F₂-phenyl) | SO₂Et | Me | CF₂CF₂CF₃ | m.p.: 133-135 (° C.) |
| b-61 | 5-(3-F-pyridin-2-yl) | SO₂Et | Me | CF₂CF₂CF₂CF₃ | m.p.: 124-126 (° C.) |
| b-62 | 5-(3-Cl-pyridin-2-yl) | SO₂Et | Me | CF₂CF₂CF₃ | m.p.: 138-140 (° C.) |
| b-63 | 5-°Pr | SO₂Et | Me | CHFCF₂CF₂CF₂CF₃ | m.p.: 135-138 (° C.) |
| b-64 | 5-(pyrimidin-2-yl) | SO₂Et | Me | CHFCF₂CF₂CF₂CF₃ | m.p.: 170-172 (° C.) |
| b-65 | 5-°Pr | SO₂Et | Me | CH₂CH(CF₃)₂ | viscous oil |
| b-66 | 5-(pyridin-2-yl) | SO₂Et | Me | CHFCF₂CF₂CF₂CF₃ | m.p.: 71-75 (° C.) |
| b-67 | 5-(pyrimidin-2-yl) | SO₂Et | Me | CHFCH₂CH₂CF₃ | white solid |
| b-68 | 5-(OCH₂CF₂CF₃) | SO₂Et | Me | CHFCF(CF₃)₂ | nₒ (21.6° C.) 1.4513 |
| b-69 | 5-(OCMe₂CF₃) | SO₂Et | Me | CHFCF(CF₃)₂ | m.p.: 71-75 (° C.) |
| b-70 | 5-°Pr | SO₂Et | Me | CH₂OCH₂CF₂CF₃ | m.p.: 71-75 (° C.) |
| b-71 | 5-°Pr | SO₂Et | Me | CH₂OCH₂CF₂CF₂CF₃ | m.p.: 71-75 (° C.) |
| b-72 | 5-°Pr | SO₂Et | Me | CH₂OCF₂CHFOCF₃ | viscous oil |

TABLE 3

| Compound | | |
|---|---|---|
| No. | Structure | Physical properties |
| c-1 | 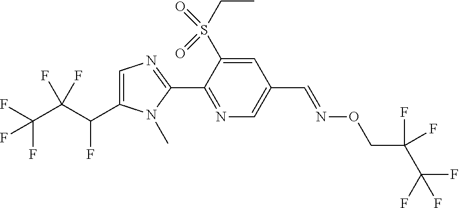 [SMILES: CCS(=O)(=O)c1cc(/C=N/OCC(F)(F)C(F)(F)F)cnc1-c1ncc(C(F)C(F)(F)C(F)(F)F)n1C] | m.p.: 140-142(° C.) |
| c-2 | 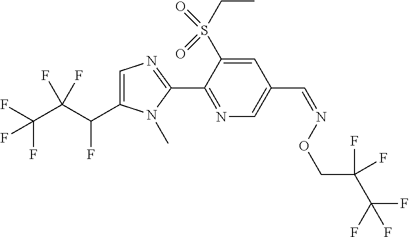 [SMILES: CCS(=O)(=O)c1cc(/C=N\OCC(F)(F)C(F)(F)F)cnc1-c1ncc(C(F)C(F)(F)C(F)(F)F)n1C] | white solid |
| c-3 | 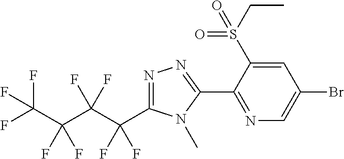 [SMILES: CCS(=O)(=O)c1cc(Br)cnc1-c1nnc(C(F)(F)C(F)(F)C(F)(F)C(F)(F)F)n1C] | m.p.: 74-76(° C.) |
| c-4 | 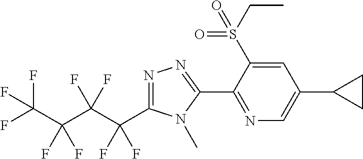 [SMILES: CCS(=O)(=O)c1cc(C2CC2)cnc1-c1nnc(C(F)(F)C(F)(F)C(F)(F)C(F)(F)F)n1C] | m.p.: 126-128(° C.) |
| c-5 | 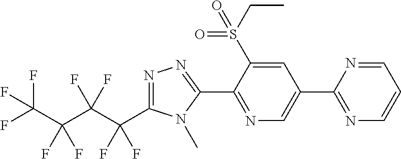 [SMILES: CCS(=O)(=O)c1cc(-c2ncccn2)cnc1-c1nnc(C(F)(F)C(F)(F)C(F)(F)C(F)(F)F)n1C] | m.p.: 151-153(° C.) |
| c-6 | 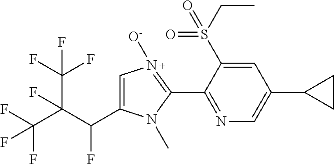 | m.p.: 186-191(° C.) |
| c-7 | 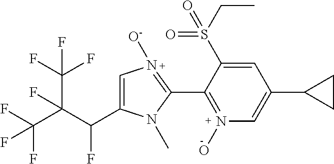 | m.p.: 203-205(° C.) |
| c-8 | 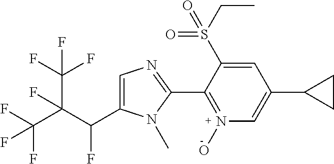 | m.p.: 174-178(° C.) |
| c-9 | 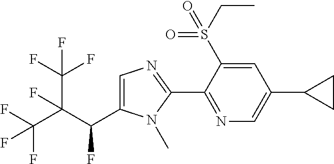 [SMILES: CCS(=O)(=O)c1cc(C2CC2)cnc1-c1ncc([C@@H](F)C(F)(C(F)(F)F)C(F)(F)F)n1C] | |
| c-10 | 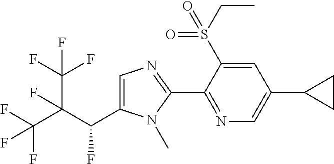 [SMILES: CCS(=O)(=O)c1cc(C2CC2)cnc1-c1ncc([C@H](F)C(F)(C(F)(F)F)C(F)(F)F)n1C] | |
| c-11 | 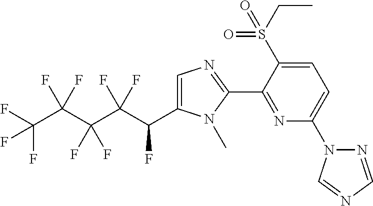 [SMILES: CCS(=O)(=O)c1ccc(-n2cncn2)nc1-c1ncc([C@@H](F)C(F)(F)C(F)(F)C(F)(F)C(F)(F)F)n1C] | |
| c-12 | 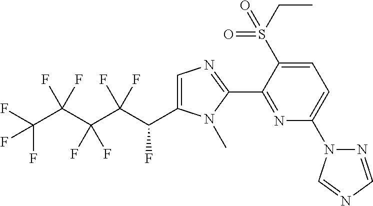 [SMILES: CCS(=O)(=O)c1ccc(-n2cncn2)nc1-c1ncc([C@H](F)C(F)(F)C(F)(F)C(F)(F)C(F)(F)F)n1C] | |
| c-13 | 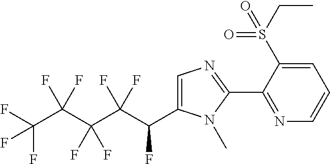 [SMILES: CCS(=O)(=O)c1cccnc1-c1ncc([C@@H](F)C(F)(F)C(F)(F)C(F)(F)C(F)(F)F)n1C] | |
| c-14 | 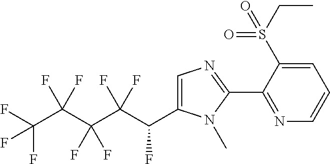 [SMILES: CCS(=O)(=O)c1cccnc1-c1ncc([C@H](F)C(F)(F)C(F)(F)C(F)(F)C(F)(F)F)n1C] | |
| c-15 | 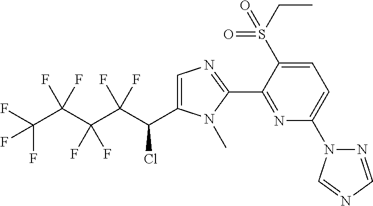 [SMILES: CCS(=O)(=O)c1ccc(-n2cncn2)nc1-c1ncc([C@@H](Cl)C(F)(F)C(F)(F)C(F)(F)C(F)(F)F)n1C] | |
| c-16 | 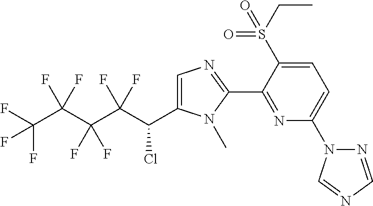 [SMILES: CCS(=O)(=O)c1ccc(-n2cncn2)nc1-c1ncc([C@H](Cl)C(F)(F)C(F)(F)C(F)(F)C(F)(F)F)n1C] | |
| c-17 | 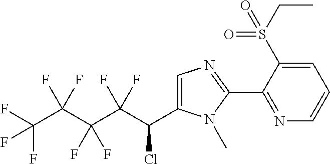 [SMILES: CCS(=O)(=O)c1cccnc1-c1ncc([C@@H](Cl)C(F)(F)C(F)(F)C(F)(F)C(F)(F)F)n1C] | |
| c-18 | 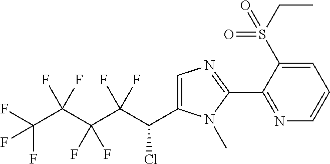 [SMILES: CCS(=O)(=O)c1cccnc1-c1ncc([C@H](Cl)C(F)(F)C(F)(F)C(F)(F)C(F)(F)F)n1C] | |
| c-19 | 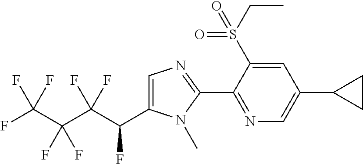 [SMILES: CCS(=O)(=O)c1cc(C2CC2)cnc1-c1ncc([C@@H](F)C(F)(F)C(F)(F)C(F)(F)F)n1C] | |
| c-20 | 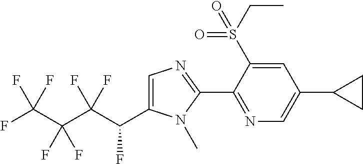 [SMILES: CCS(=O)(=O)c1cc(C2CC2)cnc1-c1ncc([C@H](F)C(F)(F)C(F)(F)C(F)(F)F)n1C] | |
| c-21 | 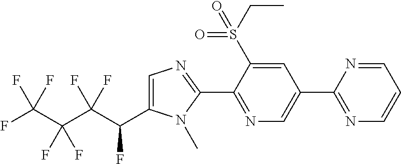 [SMILES: CCS(=O)(=O)c1cc(-c2ncccn2)cnc1-c1ncc([C@@H](F)C(F)(F)C(F)(F)C(F)(F)F)n1C] | |
| c-22 | 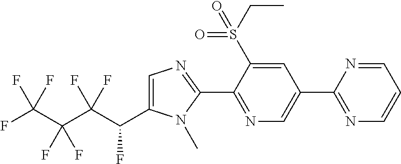 [SMILES: CCS(=O)(=O)c1cc(-c2ncccn2)cnc1-c1ncc([C@H](F)C(F)(F)C(F)(F)C(F)(F)F)n1C] | |
| c-23 | 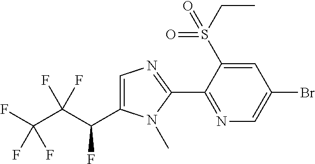 [SMILES: CCS(=O)(=O)c1cc(Br)cnc1-c1ncc([C@@H](F)C(F)(F)C(F)(F)F)n1C] | |
| c-24 | 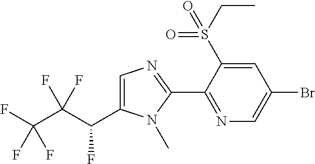 [SMILES: CCS(=O)(=O)c1cc(Br)cnc1-c1ncc([C@H](F)C(F)(F)C(F)(F)F)n1C] | |
| c-25 | 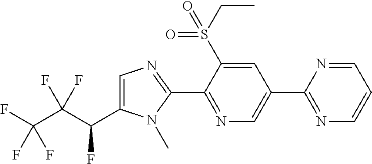 [SMILES: CCS(=O)(=O)c1cc(-c2ncccn2)cnc1-c1ncc([C@@H](F)C(F)(F)C(F)(F)F)n1C] | |
| c-26 | 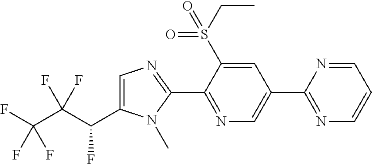 [SMILES: CCS(=O)(=O)c1cc(-c2ncccn2)cnc1-c1ncc([C@H](F)C(F)(F)C(F)(F)F)n1C] | |
| c-27 | 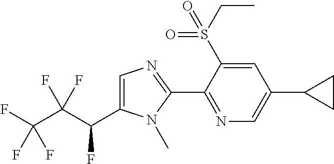 [SMILES: CCS(=O)(=O)c1cc(C2CC2)cnc1-c1ncc([C@@H](F)C(F)(F)C(F)(F)F)n1C] | |
| c-28 | 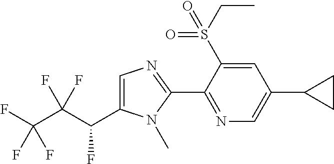 [SMILES: CCS(=O)(=O)c1cc(C2CC2)cnc1-c1ncc([C@H](F)C(F)(F)C(F)(F)F)n1C] | |
| c-29 | 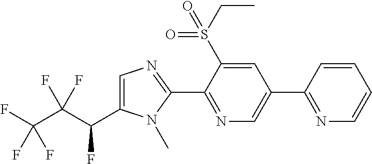 [SMILES: CCS(=O)(=O)c1cc(-c2ccccn2)cnc1-c1ncc([C@@H](F)C(F)(F)C(F)(F)F)n1C] | |
| c-30 | 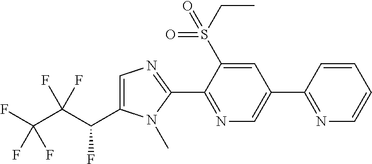 [SMILES: CCS(=O)(=O)c1cc(-c2ccccn2)cnc1-c1ncc([C@H](F)C(F)(F)C(F)(F)F)n1C] | |
| c-31 | 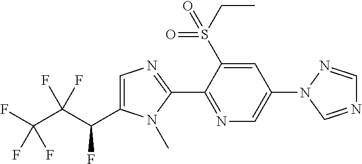 [SMILES: CCS(=O)(=O)c1cc(-n2cncn2)cnc1-c1ncc([C@@H](F)C(F)(F)C(F)(F)F)n1C] | |
| c-32 | 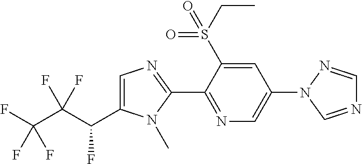 [SMILES: CCS(=O)(=O)c1cc(-n2cncn2)cnc1-c1ncc([C@H](F)C(F)(F)C(F)(F)F)n1C] | |
| c-33 | 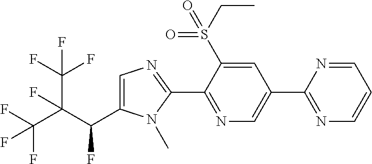 [SMILES: CCS(=O)(=O)c1cc(-c2ncccn2)cnc1-c1ncc([C@@H](F)C(F)(C(F)(F)F)C(F)(F)F)n1C] | |
| c-34 | 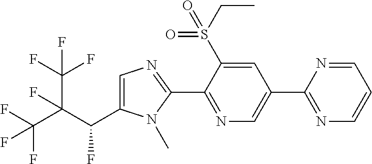 [SMILES: CCS(=O)(=O)c1cc(-c2ncccn2)cnc1-c1ncc([C@H](F)C(F)(C(F)(F)F)C(F)(F)F)n1C] | |
| c-35 | 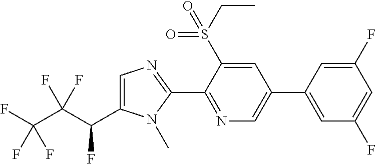 [SMILES: CCS(=O)(=O)c1cc(-c2cc(F)cc(F)c2)cnc1-c1ncc([C@@H](F)C(F)(F)C(F)(F)F)n1C] | |
| c-36 | 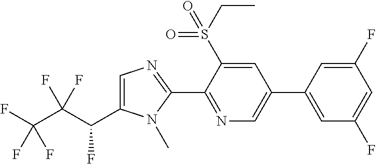 [SMILES: CCS(=O)(=O)c1cc(-c2cc(F)cc(F)c2)cnc1-c1ncc([C@H](F)C(F)(F)C(F)(F)F)n1C] | |
| c-37 | 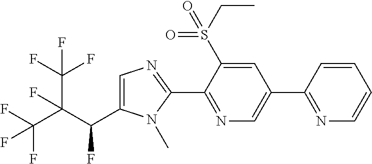 [SMILES: CCS(=O)(=O)c1cc(-c2ccccn2)cnc1-c1ncc([C@@H](F)C(F)(C(F)(F)F)C(F)(F)F)n1C] | |
| c-38 | 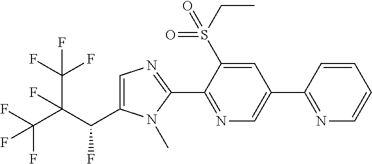 [SMILES: CCS(=O)(=O)c1cc(-c2ccccn2)cnc1-c1ncc([C@H](F)C(F)(C(F)(F)F)C(F)(F)F)n1C] | |
| c-39 | 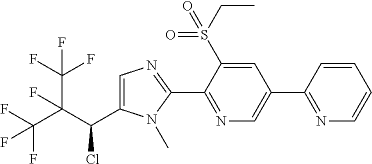 [SMILES: CCS(=O)(=O)c1cc(-c2ccccn2)cnc1-c1ncc([C@@H](Cl)C(F)(C(F)(F)F)C(F)(F)F)n1C] | |
| c-40 | 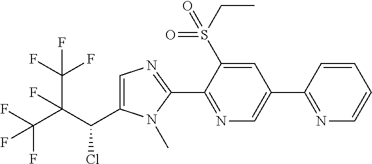 [SMILES: CCS(=O)(=O)c1cc(-c2ccccn2)cnc1-c1ncc([C@H](Cl)C(F)(C(F)(F)F)C(F)(F)F)n1C] | |
| c-41 | 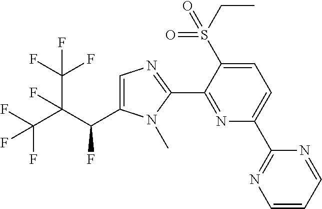 [SMILES: CCS(=O)(=O)c1ccc(-c2ncccn2)nc1-c1ncc([C@@H](F)C(F)(C(F)(F)F)C(F)(F)F)n1C] | |
| c-42 | 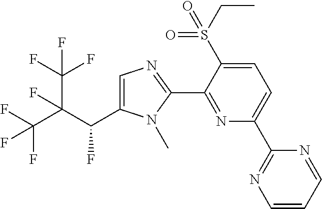 [SMILES: CCS(=O)(=O)c1ccc(-c2ncccn2)nc1-c1ncc([C@H](F)C(F)(C(F)(F)F)C(F)(F)F)n1C] | |
| c-43 | 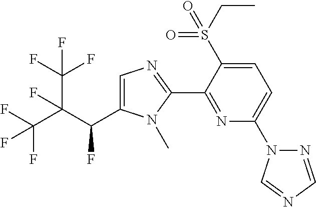 [SMILES: CCS(=O)(=O)c1ccc(-n2cncn2)nc1-c1ncc([C@@H](F)C(F)(C(F)(F)F)C(F)(F)F)n1C] | |
| c-44 | 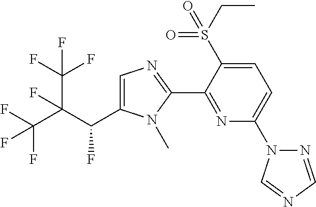 [SMILES: CCS(=O)(=O)c1ccc(-n2cncn2)nc1-c1ncc([C@H](F)C(F)(C(F)(F)F)C(F)(F)F)n1C] | |
| c-45 | 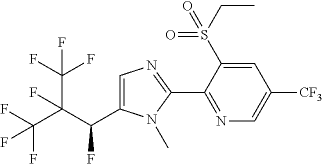 [SMILES: CCS(=O)(=O)c1cc(C(F)(F)F)cnc1-c1ncc([C@@H](F)C(F)(C(F)(F)F)C(F)(F)F)n1C] | |
| c-46 | 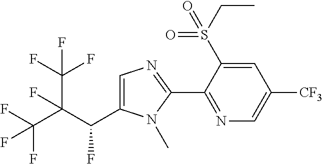 [SMILES: CCS(=O)(=O)c1cc(C(F)(F)F)cnc1-c1ncc([C@H](F)C(F)(C(F)(F)F)C(F)(F)F)n1C] | |
| c-47 | 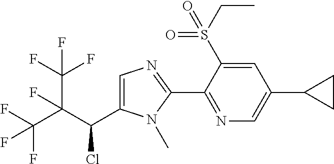 [SMILES: CCS(=O)(=O)c1cc(C2CC2)cnc1-c1ncc([C@@H](Cl)C(F)(C(F)(F)F)C(F)(F)F)n1C] | |
| c-48 | 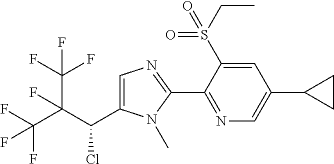 [SMILES: CCS(=O)(=O)c1cc(C2CC2)cnc1-c1ncc([C@H](Cl)C(F)(C(F)(F)F)C(F)(F)F)n1C] | |
| c-49 | 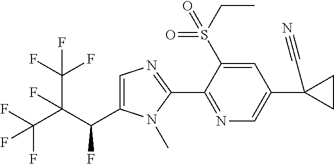 [SMILES: CCS(=O)(=O)c1cc(C2(C#N)CC2)cnc1-c1ncc([C@@H](F)C(F)(C(F)(F)F)C(F)(F)F)n1C] | |
| c-50 | 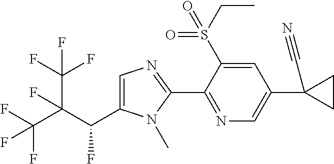 [SMILES: CCS(=O)(=O)c1cc(C2(C#N)CC2)cnc1-c1ncc([C@H](F)C(F)(C(F)(F)F)C(F)(F)F)n1C] | |
| c-51 | 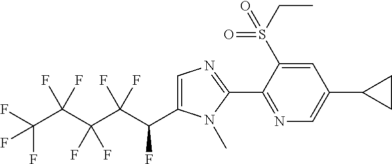 [SMILES: CCS(=O)(=O)c1cc(C2CC2)cnc1-c1ncc([C@@H](F)C(F)(F)C(F)(F)C(F)(F)C(F)(F)F)n1C] | |
| c-52 | 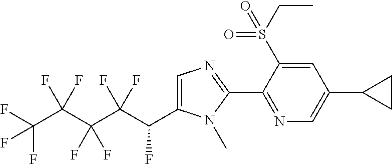 [SMILES: CCS(=O)(=O)c1cc(C2CC2)cnc1-c1ncc([C@H](F)C(F)(F)C(F)(F)C(F)(F)C(F)(F)F)n1C] | |
| c-53 | 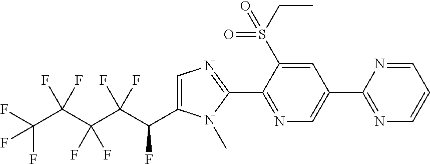 [SMILES: CCS(=O)(=O)c1cc(-c2ncccn2)cnc1-c1ncc([C@@H](F)C(F)(F)C(F)(F)C(F)(F)C(F)(F)F)n1C] | |
| c-54 | 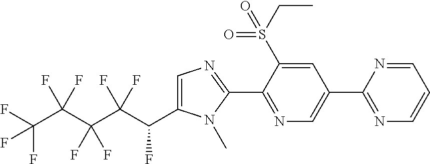 [SMILES: CCS(=O)(=O)c1cc(-c2ncccn2)cnc1-c1ncc([C@H](F)C(F)(F)C(F)(F)C(F)(F)C(F)(F)F)n1C] | |
| c-55 | 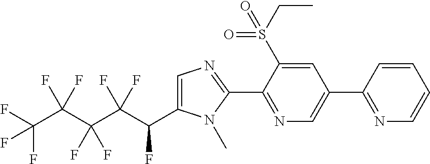 [SMILES: CCS(=O)(=O)c1cc(-c2ccccn2)cnc1-c1ncc([C@@H](F)C(F)(F)C(F)(F)C(F)(F)C(F)(F)F)n1C] | |
| c-56 | 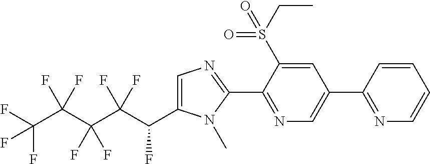 [SMILES: CCS(=O)(=O)c1cc(-c2ccccn2)cnc1-c1ncc([C@H](F)C(F)(F)C(F)(F)C(F)(F)C(F)(F)F)n1C] | |
| c-57 | 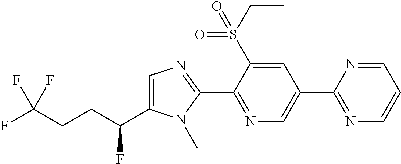 [SMILES: CCS(=O)(=O)c1cc(-c2ncccn2)cnc1-c1ncc([C@@H](F)CCC(F)(F)F)n1C] | |
| c-58 | 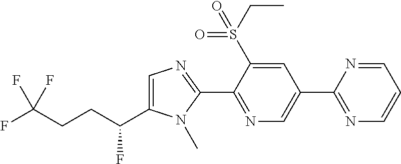 [SMILES: CCS(=O)(=O)c1cc(-c2ncccn2)cnc1-c1ncc([C@H](F)CCC(F)(F)F)n1C] | |
| c-59 | 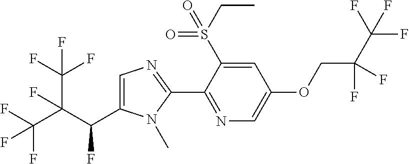 [SMILES: CCS(=O)(=O)c1cc(OCC(F)(F)C(F)(F)F)cnc1-c1ncc([C@@H](F)C(F)(C(F)(F)F)C(F)(F)F)n1C] | |
| c-60 | 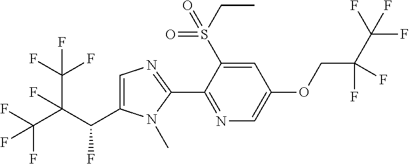 [SMILES: CCS(=O)(=O)c1cc(OCC(F)(F)C(F)(F)F)cnc1-c1ncc([C@H](F)C(F)(C(F)(F)F)C(F)(F)F)n1C] | |
| c-61 | 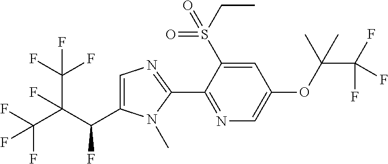 [SMILES: CCS(=O)(=O)c1cc(OC(C)(C)C(F)(F)F)cnc1-c1ncc([C@@H](F)C(F)(C(F)(F)F)C(F)(F)F)n1C] | |
| c-62 | 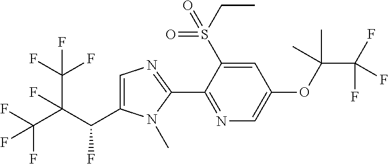 [SMILES: CCS(=O)(=O)c1cc(OC(C)(C)C(F)(F)F)cnc1-c1ncc([C@H](F)C(F)(C(F)(F)F)C(F)(F)F)n1C] | |

The ¹H-NMR data of compounds having physical properties of viscous oil, amorphous or white solid among the compounds shown in Tables 1 to 3 will be shown below. Compound No. a-10: ¹H-NMR (CDCl₃) δ: 9.55 (1H, s), 8.10 (1H, s), 7.56 (1H, s), 4.44 (3H, s), 3.93 (2H, q), 3.91 (3H, s), 1.42 (3H, t).

Compound No. b-28: ¹H-NMR (400 MHz, CDCl3) δ: 9.37 (d, 1H), 8.80 (s, 1H), 8.78 (d, 1H), 8.25 (s, 1H), 7.44 (d, 1H), 5.92 (ddd, 1H), 3.88 (q, 2H), 3.70 (s, 3H), 1.38 (t, 3H).

Compound No. b-31: ¹H-NMR (400 MHz, CDCl3) δ: 9.93 (d, 1H), 9.47 (d, 1H), 8.91 (d, 2H), 7.40 (dd, 1H), 7.37 (t, 1H), 6.10 (ddd, 1H), 3.80 (q, 2H), 3.67 (d, 3H), 1.37 (t, 3H).

Compound No. b-37: ¹H-NMR (400 MHz, CDCl3) δ: 8.83 (d, 1H), 8.31 (d, 1H), 8.19 (dd, 2H), 7.83 (dt, 1H), 7.50 (s, 1H), 7.16 (dd, 1H), 7.12 (d, 1H), 3.70 (q, 2H), 3.67 (s, 3H), 1.33 (t, 3H).

Compound No. b-40: ¹H-NMR (400 MHz, CDCl3) δ: 10.28 (s, 1H), 9.35 (d, 1H), 8.92 (d, 1H), 7.45 (d, 1H), 5.90 (ddd, 1H), 3.96-3.86 (m, 2H), 3.74 (s, 3H), 1.37 (t, 3H).

Compound No. b-56: ¹H-NMR (400 MHz, CDCl3) δ: 8.99 (1H, d), 8.19 (1H, d), 7.37 (1H, dd), 6.08 (1H, dd), 3.76 (2H, q), 3.62 (3H, s), 2.00 (2H, dd), 1.63 (2H, dd), 1.31 (3H, t).

Compound No. b-65: ¹H-NMR (400 MHz, CDCl3) δ: 8.67 (1H, d), 8.01 (1H, d), 7.01 (1H, s), 3.71 (2H, q), 3.49 (3H, s), 3.30-3.17 (3H, m), 2.10-2.03 (1H, m), 1.28 (3H, t), 1.24-1.19 (2H, m), 0.93-0.88 (2H, m).

Compound No. b-67: ¹H-NMR (400 MHz, CDCl3) δ: 9.91 (1H, d), 9.47 (1H, d), 8.91 (2H, d), 7.36 (1H, t), 7.23 (1H, d), 5.59 (1H, ddd), 3.92-3.80 (2H, m), 3.69 (3H, s), 2.56-2.33 (4H, m), 1.38 (3H, t).

Compound No. b-72: ¹H-NMR (400 MHz, CDCl3) δ: 8.68 (1H, d), 8.02 (1H, d), 7.21 (1H, s), 5.76 (1H, dt), 5.09 (2H, s), 3.74 (2H, q), 3.55 (3H, s), 2.11-2.05 (1H, m), 1.30 (3H, t), 1.25-1.16 (2H, m), 0.96-0.89 (2H, m).

Compound No. c-2: ¹H-NMR (400 MHz, CDCl3) δ:9.31 (d, 1H), 8.89 (d, 1H), 7.58 (s, 1H), 7.43 (d, 1H), 5.91 (ddd, 1H), 4.79 (t, 2H), 3.89-3.78 (m, 2H), 3.71 (s, 3H), 1.34 (t, 3H).

### [Biological Test]

Test Examples given below show that the heteroaryl azole compound of the present invention is useful as an active ingredient for pest control agents and ectoparasite control agents. The term “part” is based on weight.

### (Preparation of Test Emulsion)

5 parts of the heteroaryl azole compound of the present invention, 93.6 parts of dimethylformamide, and 1.4 parts of polyoxyethylene alkyl aryl ether were mixed and dissolved to prepare emulsion (I) containing 5% of the active ingredient.

For a control, 98.5 parts of dimethylformamide and 1.5 parts of polyoxyethylene alkyl aryl ether were mixed and dissolved to prepare emulsion (II).

An insecticidal rate was calculated according to the following expression:

Insecticidal rate (%)=(The number of dead insects/The number of tested insects)×100

### (Test Example 1) Test of Efficacy on Mythimna Separata

0.8 g of commercially available artificial feed (Insecta LFS, manufactured by Nosan Corp.) and 1 μl of the emulsion (I) were well mixed to obtain test feed.

A plastic test container (capacity: 1.4 ml) was packed with 0.2 g of the test feed per treatment plot. Then, two second instar larvae of Mythimna separata were inoculated to each treatment plot. A plastic lid was put on the test container so as to prevent escape of the second instar larvae of Mythimna separata. The container was placed in a thermostat chamber of 25° C. On the fifth day, the insecticidal rate and the food intake were examined. The test was conducted in duplicate.

The insecticidal rate and the food intake of a control plot were examined in the same way as in Test Example 1 except that the emulsion (I) was changed to the emulsion (II).

Compounds of compound Nos. a-1, a-2, a-6, a-8, a-9, b-1, b-2, b-3, b-4, b-7, b-8, b-9, b-10, b-11, b-12, b-13, b-15, b-16, b-17, b-18, b-20, b-22, b-23, b-24, b-25, b-26, b-27, b-29, b-32, b-34, b-35, b-36, b-39, b-41, b-42, b-43, b-44, b-45, b-52, b-53, b-54, b-55, b-57, b-58, b-59, b-60, b-61, b-62, b-63, b-64, b-66, b-68, b-69, c-1, c-5 and c-8 were tested for their efficacy on Mythimna separata. All the compounds had an insecticidal rate of 100% for Mythimna separata or a food intake of 10% or less as compared with the control plot. As is evident, the heteroaryl azole compound of the present invention is effective for Mythimna separata.

### (Test Example 2) Test of Efficacy on Mythimna Separata

The emulsion (I) was diluted with water such that the concentration of the compound of the present invention was 125 ppm. Corn leaves were dipped in the dilution for 30 seconds. The resulting corn leaves were placed in a petri dish, and five second instar larvae of Mythimna separata were released. The petri dish was placed in a thermostat chamber having a temperature of 25° C. and a humidity of 60%. Life and death were determined after 6 days from the release of the insects, and the insecticidal rate was calculated. The test was conducted in duplicate.

Compounds of compound Nos. a-2, b-4, b-18, b-19 and b-21 were tested for their efficacy on Mythimna separata. All the compounds exhibited an insecticidal rate of 80% or more for Mythimna separata.

### (Test Example 3) Test of Efficacy on Plutella xylostella

The emulsion (I) was diluted with water such that the concentration of the compound of the present invention was 125 ppm. Cabbage leaves were dipped in the dilution for 30 seconds. The resulting cabbage leaves were placed in a petri dish. Five second instar larvae of Plutella xylostella were released thereto. The petri dish was placed in a thermostat chamber having a temperature of 25° C. and a humidity of 60%. Life and death were determined after 3 days from the release of the insects, and the insecticidal rate was calculated. The test was conducted in duplicate.

Compounds of compound Nos. a-1, a-2, a-4, a-5, a-6, a-10, b-1, b-4, b-8, b-9, b-10, b-13, b-15, b-16, b-18, b-19, b-20, b-21, b-22, b-24, b-25, b-27, b-29, b-31, b-34, b-36, b-37, b-41, b-42, b-43, b-44, b-45, b-47, b-48, b-51, b-52, b-54, b-55, b-56, b-58, b-60, b-61, b-62, b-64, b-67, b-69, c-1, c-4 and c-5 were tested for their efficacy on Plutella xylostella. All the compounds exhibited an insecticidal rate of 80% or more for Plutella xylostella.

### (Test Example 4) Test of Efficacy on Spodoptera Litura

The emulsion (I) was diluted with water such that the concentration of the compound of the present invention was 125 ppm. Cabbage leaves were dipped in the dilution for 30 seconds. The resulting cabbage leaves were placed in a petri dish. Five second instar larvae of Spodoptera litura were released thereto. The petri dish was placed in a thermostat chamber having a temperature of 25° C. and a humidity of 60%. Life and death were determined after 6 days from the release of the insects, and the insecticidal rate was calculated. The test was conducted in duplicate.

Compounds of compound Nos. b-18, b-20, b-21 and b-were tested for their efficacy on Spodoptera litura. All the compounds exhibited an insecticidal rate of 80% or more for Spodoptera litura.

### (Test Example 5) Test of Efficacy on Aphis Craccivora

Seedlings of black-eyed peas were raised in 10-cm pots. Aphis craccivora nymphs were inoculated onto primary leaves. The emulsion (I) was diluted with water such that the concentration of the compound of the present invention was 125 ppm. The dilution was sprayed to the black-eyed peas parasitized by the Aphis craccivora nymphs. The black-eyed peas were placed in a thermostat chamber having a temperature of 25° C. and a humidity of 60%. Life and death of Aphis craccivora were determined after 4 days from the spraying, and the insecticidal rate was calculated. The test was conducted in duplicate.

Compounds of compound Nos. a-1, a-2, a-6, a-9, b-1, b-2, b-3, b-4, b-7, b-8, b-9, b-11, b-12, b-15, b-16, b-17, b-18, b-20, b-22, b-23, b-24, b-25, b-26, b-27, b-29, b-32, b-35, b-36, b-38, b-40, b-41, b-42, b-43, b-44, b-45, b-52, b-53, b-54, b-55, b-57, b-58, b-59, b-60, b-61, b-62, b-63, b-64, b-66, b-69, b-70, b-71, c-7 and c-8 were tested for their efficacy on Aphis craccivora. All the compounds exhibited an insecticidal rate of 80% or more for Aphis craccivora.

### (Test Example 6) Test of Efficacy on Phyllotreta Striolata

The emulsion (I) was diluted with water such that the concentration of the compound of the present invention was 125 ppm to prepare a test chemical. The test chemical was sprayed to Qing geng cai seedlings (at the seventh true leaf stage) planted in 10-cm pots. The Qing geng cai seedlings were dried in air and then placed in a plastic cup. Ten Phyllotreta striolata adults were released thereto. The plastic cup was stored in a thermostat chamber having a temperature of 25° C. and a humidity of 65%. Life and death were determined after 7 days from the release of the insects, and the insecticidal rate was calculated. The test was conducted in duplicate.

Compounds of compound Nos. a-1, a-2, a-6, b-1, b-4, b-8, b-9, b-18, b-19, b-21, b-22, b-24, b-29, b-31, b-34, b-37, b-43, b-48, b-52, b-55, b-58, b-59, b-61, b-62, b-69, c-1, c-2 and c-5 were tested for their efficacy on Phyllotreta striolata adults. All the compounds exhibited an insecticidal rate of 80% or more for Phyllotreta striolata adults.

### (Test Example 7) Test of Efficacy on Phyllotreta striolata

The emulsion (I) was diluted with water such that the concentration of the compound of the present invention was 2 ppm to prepare a test chemical. The test chemical was sprayed to Qing geng cai seedlings (at the seventh true leaf stage) planted in 10-cm pots. The Qing geng cai seedlings were dried in air and then placed in a plastic cup. Ten Phyllotreta striolata adults were released thereto. The plastic cup was stored in a thermostat chamber having a temperature of 25° C. and a humidity of 65%. Life and death were determined after 7 days from the release of the insects, and the insecticidal rate was calculated. The test was conducted in duplicate.

Compounds of compound Nos. a-1, a-2, a-6, b-1, b-4, b-8, b-18, b-19, b-21, b-24, b-29, b-31, b-34, b-43, b-48, b-52, b-55, b-58, b-59, b-61, b-62 and c-1 were tested for their efficacy on Phyllotreta striolata adults. All the compounds exhibited an insecticidal rate of 80% or more for Phyllotreta striolata adults.

### (Test Example 8) Test of Efficacy on Nilaparvata lugens

The emulsion (I) was diluted with water such that the concentration of the compound of the present invention was 125 ppm. Young seedlings of rice were dipped in the dilution for 30 seconds. The young seedlings of rice were dried in air and then placed in a plastic case. Five second instar larvae of Nilaparvata lugens were released thereto. The plastic case was stored in a thermostat chamber having a temperature of 25° C. and a humidity of 65%. Life and death were determined after 7 days from the inoculation, and the insecticidal rate was calculated. The test was conducted in duplicate.

Compounds of compound Nos. a-1, a-2, a-3, a-4, a-9, b-3, b-4, b-7, b-9, b-15, b-17, b-19, b-28, b-34, b-46, b-47, b-48, b-51 and b-67 were tested for their efficacy on Nilaparvata lugens. All the compounds exhibited an insecticidal rate of 80% or more for Nilaparvata lugens.

### (Test Example 9) Test of Efficacy on Musca domestica

The compound of the present invention was diluted with acetone and added dropwise at 100 ppm per g of a cube of sugar. The cube of sugar was placed in a plastic cup. Ten female adults of Musca domestica were released, and a lid was put on the plastic cup. The plastic cup was stored at 25° C. Life and death were determined after hours from the release of the insects, and the insecticidal rate was calculated. The test was conducted in duplicate.

A compound of compound No. b-4 was tested for its efficacy on Musca domestica. The compound exhibited an insecticidal rate of 80% or more for Musca domestica.

All the compounds selected at random from among the heteroaryl azole compounds of the present invention exerted the effect as described above. It may therefore be understood that the heteroaryl azole compound of the present invention, including unillustrated compounds, is a compound having an effect such as a pest control effect, particularly, a miticidal or insecticidal effect. It may also be understood that the heteroaryl azole compound of the present invention is a compound also having an effect on parasites harmful to humans and animals, such as ectoparasites.

1. A compound represented by the formula (I), an N-oxide compound, stereoisomer, tautomer or hydrate thereof or a salt of any of these compounds: 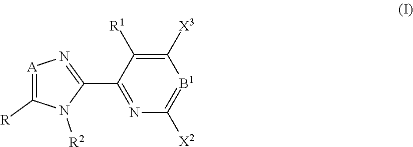
wherein
A represents CH or a nitrogen atom;
B¹ represents CX¹ or a nitrogen atom;
X¹, X² and X³ each independently represent a hydrogen atom, a substituted or unsubstituted C1-6 alkyl group, a substituted or unsubstituted C2-6 alkenyl group, a substituted or unsubstituted C2-6 alkynyl group, a hydroxy group, a substituted or unsubstituted C1-6 alkoxy group, a substituted or unsubstituted C1-6 alkoxycarbonyl group, a formyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-10 aryl group, a substituted or unsubstituted 3- to 6-membered heterocyclyl group, a substituted or unsubstituted C6-10 aryloxy group, a substituted or unsubstituted 5- or 6-membered heteroaryloxy group, a substituted or unsubstituted amino group, a substituted or unsubstituted aminocarbonyl group, a substituted or unsubstituted hydrazinyl group, a nitro group, a cyano group, a halogeno group, a group represented by the formula: —CR³═N—OR⁴, a group represented by the formula:
—N═CHNR⁵R⁶, or a group represented by the formula:
—N═S(O)_{q}R⁷R⁸;
R³ and R⁴ each independently represent a hydrogen atom, a C1-6 alkyl group, or a C1-6 haloalkyl group;
R⁵, R⁶, R⁷ and R⁸ each represent a C1-6 alkyl group;
q represents 0 or 1;
R¹ represents a substituted or unsubstituted C1-6 alkylthio group, a substituted or unsubstituted C1-6 alkylsulfinyl group, or a substituted or unsubstituted C1-6 alkylsulfonyl group;
R² represents a substituted or unsubstituted C1-6 alkyl group; and
R represents a substituted or unsubstituted C1-6 alkyl group.

2. The compound according to claim 1, an N-oxide compound, stereoisomer, tautomer or hydrate thereof or a salt of any of these compounds, wherein the formula (I) is the formula (II): 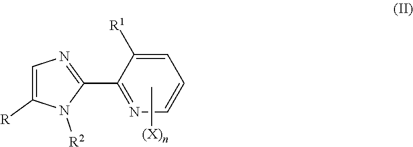
wherein
R¹, R², and R have the same meanings as described in the formula (I);
X represents a substituted or unsubstituted C1-6 alkyl group, a substituted or unsubstituted C2-6 alkenyl group, a substituted or unsubstituted C2-6 alkynyl group, a hydroxy group, a substituted or unsubstituted C1-6 alkoxy group, a substituted or unsubstituted C1-6 alkoxycarbonyl group, a formyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-10 aryl group, a substituted or unsubstituted 3- to 6-membered heterocyclyl group, a substituted or unsubstituted C6-10 aryloxy group, a substituted or unsubstituted 5- or 6-membered heteroaryloxy group, a substituted or unsubstituted amino group, a substituted or unsubstituted aminocarbonyl group, a substituted or unsubstituted hydrazinyl group, a nitro group, a cyano group, a halogeno group, a group represented by the formula: —CR³═N—OR⁴, a group represented by the formula: —N═CHNR⁵R⁶, or a group represented by the formula: —N═S(O)_{q}R⁷R⁸;
R³ and R⁴ each independently represent a hydrogen atom, a C1-6 alkyl group, or a C1-6 haloalkyl group;
R⁵, R⁶, R⁷ and R⁸ each represent a C1-6 alkyl group;
q represents 0 or 1; and
n represents a chemically acceptable number of X and is any integer of 0 to 3, and when n is 2 or larger, each of the X is the same or different.

3. The compound according to claim 1, an N-oxide compound, stereoisomer, tautomer or hydrate thereof or a salt of any of these compounds, wherein the formula (I) is the formula (III): 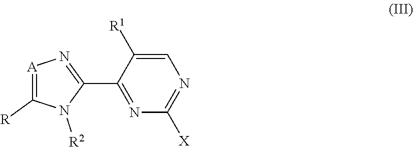
wherein
A, R¹, R², and R have the same meanings as described in the formula (I); and
X has the same meaning as described in the formula (II).

4. The compound according to claim 1, an N-oxide compound, stereoisomer, tautomer or hydrate thereof or a salt of any of these compounds, wherein in the formula (I), R represents a C1-6 haloalkyl group.

5. A pest control agent comprising at least one active ingredient selected from the group consisting of a compound according to claim 1, an N-oxide compound, stereoisomer, tautomer and hydrate thereof and a salt of any of these compounds.

6. An insecticide or miticide comprising at least one active ingredient selected from the group consisting of a compound according to claim 1, an N-oxide compound, stereoisomer, tautomer and hydrate thereof and a salt of any of these compounds.

7. An ectoparasite control agent comprising at least one active ingredient selected from the group consisting of a compound according to claim 1, an N-oxide compound, stereoisomer, tautomer and hydrate thereof and a salt of any of these compounds.

8. An endoparasite control agent or expellant comprising at least one active ingredient selected from the group consisting of a compound according to claim 1, an N-oxide compound, stereoisomer, tautomer and hydrate thereof and a salt of any of these compounds.

9. A seed treatment agent or vegetative propagation organ treatment agent comprising at least one active ingredient selected from the group consisting of a compound according to claim 1, an N-oxide compound, stereoisomer, tautomer and hydrate thereof and a salt of any of these compounds.

10. A granular agrochemical composition for paddy rice seedling nursery box treatment comprising at least one active ingredient selected from the group consisting of a compound according to claim 1, an N-oxide compound, stereoisomer, tautomer and hydrate thereof and a salt of any of these compounds.

11. A soil treatment agent comprising at least one active ingredient selected from the group consisting of a compound according to claim 1, an N-oxide compound, stereoisomer, tautomer and hydrate thereof and a salt of any of these compounds.

12. A bait agent comprising at least one active ingredient selected from the group consisting of a compound according to claim 1, an N-oxide compound, stereoisomer, tautomer and hydrate thereof and a salt of any of these compounds.

13. A plant growth promoter comprising at least one active ingredient selected from the group consisting of a compound according to claim 1, an N-oxide compound, stereoisomer, tautomer and hydrate thereof and a salt of any of these compounds.
